(19) United States
(12) Patent Application Publication
(10) Pub. No.: US 2003/0203888 A1
(43) Pub. Date: Oct. 30, 2003

(54) Chromophores

(76) Inventors: Ross William Boyle, Beverley (GB); Oliver James Clarke, Beverley (GB); Jonathan Mark Sutton, Colchester (GB); John Greenman, Cottingham (GB)

Correspondence Address: BIRCH STEWART KOLASCH & BIRCH, PO BOX 747, FALLS CHURCH, VA, 22040-0747, US

(21) Appl. No.: 10/312,347

(22) PCT Filed: Jun. 26, 2001
(86) PCT No.: PCT/GB01/02846

(30) Foreign Application Priority Data
Jun. 6, 2001 (GB) 0113784.3

(51) Int. Cl. C07D 487/22; A61K 31/555; A61K 31/409

(52) U.S. Cl. 514/185; 514/410; 540/145

(57) ABSTRACT

The present invention relates to novel porphyrin and porphyrin-based chromophores and sets of porphyrin and porphyrin-based chromophores, which may be particularly useful in a range of photodynamic applications, including photochemotherapy and fluorescence analysis and imaging. In particular, the present invention provides new and useful porphyrin, chlorin and bacteriochlorin chromophores; methods for the production of such chromophores; and methods for the use of such chromophores in analysis and in medicine.

The present invention relates to novel porphyrin and porphyrin-based chromophores and sets of porphyrin and porphyrin-based chromophores, which may be particularly useful in a range of photodynamic applications, including photochemotherapy and fluorescence analysis and imaging.

The importance of porphyrin and porphyrin-based chromophores both as research tools, for example in fluorescence-activated cell sorting (FACS), and as therapeutic agents in photodynamic therapy (PDT) for bringing about the death of targeted cells in vivo, is widely recognised in the art. Each of these applications is dependent on the ability of the chromophore to be excited by incident light to a singlet excited state, and to decay to a lower energy state with the consequent emission of energy. This energy may be emitted in the form of fluorescent light at a specific wavelength, thereby enabling a cell or biostructure attached to the decaying chromophore to be visualised, and/or sorted by FACS. Alternatively, the energy of excitation may be dissipated by initial conversion of the singlet chromophore into the triplet excited state, followed by the transfer of energy to another triplet such as dioxygen, with the consequent formation of singlet oxygen. Singlet oxygen is a powerful cytotoxic agent, and hence where this latter process occurs in or in the immediate vicinity of a cell, it will usually result in the death of that cell. Accordingly, the chromophore can be exploited both for its fluorescent properties, and for its ability to act as a photosensitiser.

Evidently, for the purposes of fluorescence imaging or analysis, or PDT, some degree of control over the localisation of the chromophore in vitro or in vivo is a prerequisite. This is particularly important in photodynamic therapy, as the typical sphere of radiation of singlet oxygen produced by decay of a chromophore is no more than 0.1 μm in diameter, so that in order to bring about the death of a target cell, the chromophore must usually be positioned immediately alongside, or preferably within, that cell.

Hitherto, however, few attempts have been made to control the targeting of porphyrin photosensitisers to particular target cells in vivo for the purposes of PDT. Instead, reliance has typically been placed on the inherent tendency of porphyrins to accumulate in tumours in the absence of lymphatic drainage from tumour structures. Phototrin®, a photosensitising agent comprising a mixture of porphyrin structures derived from hematoporphyrin-IX by treatment with acids which is commercially used in the treatment of carcinomas and sarcomas, is, for example, conventionally administered systemically to patients without any targeting vehicle or means. This is evidently undesirable, as incorrect localisation of the photosensitiser will not only decrease the efficiency of the photochemotherapy, but may also result in the death of healthy cells.

Efforts have been made to achieve the specific attachment of chromophores to biological targets in vitro, in particular for the purposes of FACS and fluorescence imaging, by covalently conjugating the chromophores to suitable protein delivery molecules. This approach has however been subject to various problems. Firstly, the degree of background fluorescence caused by non-specific binding of porphyrin chromophores to cell surfaces has proved difficult to reduce. Secondly, it has been found that the attachment of a chromophore to a protein molecule can result in a significant degree of excited state quenching by the proximate protein, which will clearly reduce the efficacy of the chromophore as a marker or in targeted photodynamic applications.

A reduction in these effects remains a desirable objective.

According to one aspect of the present invention therefore, there is provided a porphyrin chromophore of formula (I) below:

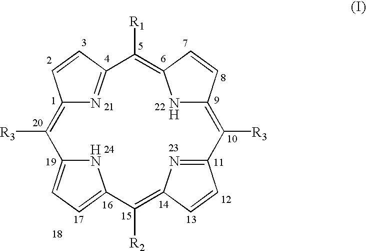

or a chlorin chromophore of any of formulas (II), (III), (IV), or (V) below:

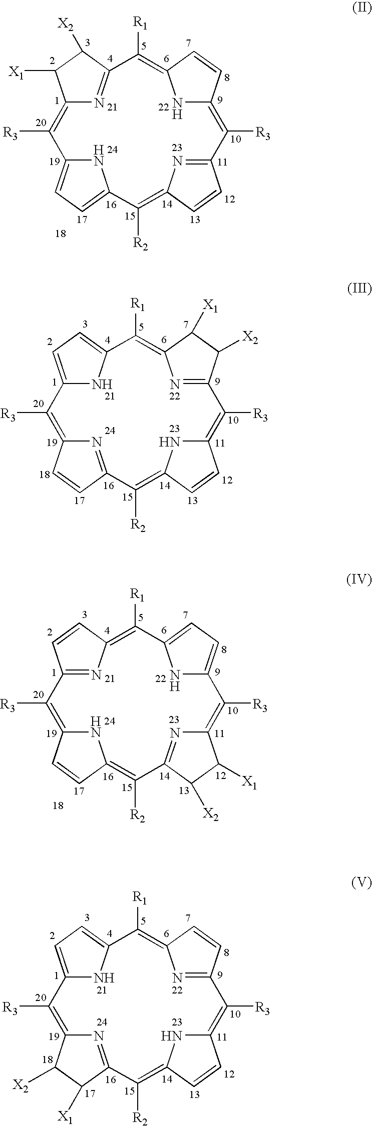

or a bacteriochlorin chromophore of anyof formulas (VI) and (VII) below:

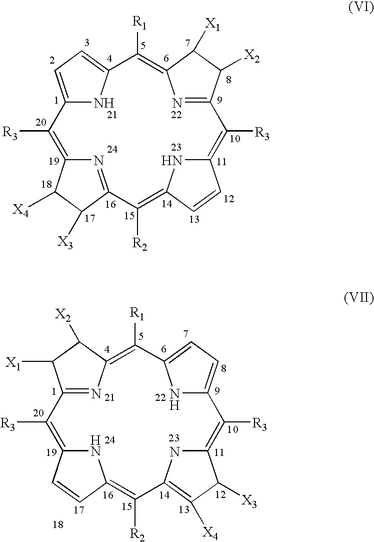

wherein R₁ is an aryl moiety which is linked to a conjugating group Z which is capable of conjugating the chroinophore to a polypeptide molecule for delivering said chromophore to a specific biological target in vitro or in vivo; R₂ is a hydrophilic aryl moiety; R₃ is H or a hydrophilic aryl or hydrophilic non-aromatic moiety; and each of X₁, X₂, X₃ and X₄ is independently selected from H, OH, halogen, C₁₋₃ alkyl and OC₁₋₃ alkyl, or X₁ and X₂ and/or X₃ and X₄ together form a bridging moiety selected from O, CH₂, CH C₁₋₃ alkyl, or C(C₁₋₃ alkyl)₂, such that X₁ and X₂ and/or X₃ and X₄ with the adjacent C—C bond form an epoxide or cyclopropanyl structure, wherein each of said R₁, R₂ and R₃ is optionally further substituted one or more times by —OH, —CN, —NO₂, halogen, -T or —OT, where T is a C₁-C₁₅ alkyl, cycloalkyl or aryl group or a hydroxylated, halogenated, sulphated, sulphonated or aminated derivative thereof or a carboxylic acid, ester, ether, polyether, amide, aldehyde or ketone derivative thereof.

It has been found that the inclusion of one or more hydrophilic substituents around the core of a chromophore in accordance with the invention results in enhanced solubility in basic buffer/DMSO or DMf co-solutions which are commonly used in protein bioconjugation. Increased hydrophilicity also produces a marked reduction in the tendency of the chromophore to bind non-covalently to proteins. Where the chromophore is to be conjugated to a targeting protein such as a monoclonal antibody for delivery to specific cells or tissues, for example for the purposes of PDT or FACS, a decrease in non-covalent binding between the chromophore and the protein will reduce the degree of non-specific transfer of chromophore to cell surfaces, which will substantially increase the accuracy of targeting the chromophore to the cells or tissue of interest.

Accordingly, according to yet another aspect of the present invention there is provided a method for separating a mixture which comprises one or more hydrophilic chromophores each having a hydrophilic or amphiphilic moiety, and a plurality of less hydrophilic substances and/or molecules, comprising the step of introducing said mixture to a hydrophobic eluting solvent, and passing said mixture and said eluting solvent over a hydrophilic or polar solid phase, such that said one or more chromophores are arrested on said solid phase whilst said substances and/or molecules are eluted or substantially eluted from said solid phase by said eluting solvent.

Said method may, for example, comprise chromatography on a Sephadex® (dextran) column, or reverse-phase HPLC. Typically, said mixture of less hydrophilic substances and/or molecules may comprise a mixture of cells and/or membranes. Advantageously, said one or more hydrophilic chromophores include one or more chromophores in accordance with the present invention.

In some embodiments, each or some of X₁-X₄ is H. In particularly preferred embodiments, however, each of X₁-X₄ is OH. Accordingly, said chromophore may be a dihydroxychlorin of formula (II), (III), (IV) or (V) above or a tetrahydroxybacteriochlorin of formula (VI) or (VII) above. The hydrophilicity of dihydroxychlorins and 4 tetrahydroxybacteriochlorins is found to be greater than that of the corresponding porphyrins, owing to the presence of extra hydrophilic hydroxy groups around the core of the chromophore.

Preferably, said aryl moiety R₁ may comprise a phenyl ring, which phenyl ring may preferably be linked by a single bond to the macrocyclic core of said chromophore or may alternatively be linked thereto by a C₁₋₆ branched or linear alkyl chain. Advantageously, said conjugating group Z may be linked to said phenyl ring at the para (4′) position thereof.

Said conjugating group Z may comprise a group which is capable of bonding covalently to an amine group on a polypeptide molecule; such as an isocyanate, isothiocyanate, or NHS ester group. Advantageously, therefore, each of the meso substituents around said porphyrin, chlorin or bacteriochlorin should comprise no —NH—, —NH₂, —NH₂⁺— or —NH₃⁺ groups which could become covalently bonded to said conjugating group Z. This will serve to reduce the probability of internal cross-linkage within said chromophore. Said conjugating group Z may alternatively comprise any other protein conjugating group, such as —NH₂, —NH(C₁₋₆ alkyl), maleamide, iodoacetamide, ketone or aldehyde. Methods for achieving the conjugation of such groups to protein molecules are known in the art.

In especially preferred embodiments, said conjugating group Z comprises an isothiocyanato group. Isothiocyanates react readily with lysine residues to produce a stable linkage to proteins, and hence are particularly suitable for bioconjugation of chromophores in accordance with the invention.

Said conjugating group Z may be linked directly to said aryl-moiety R₁ by a single bond. Alternatively, said conjugating group Z may be linked to said aryl moiety R₁ by a linking moiety having a relatively high degree of inflexibility and/or steric hindrance. Said linking moiety may, for example, comprise a chain of fused or linked cycloalkyl and/or cycloaryl ring structures having a total molecular weight no greater than 1000 gmol⁻¹. In particular, said linking moiety may comprise an anthracene, acridine, anthranil, naphthyl or naphthalene moiety, or a polyacetylene, phenylacetylene, or polyphenylacetylene moiety. When said chromophore is conjugated by said conjugating group Z to a polypeptide molecule, therefore, said linking moiety can serve to keep the photoactive core of said chromophore apart from said polypeptide, thereby helping to reduce the degree of fluorescence quenching which may be caused by said polypeptide when said chromophore is caused to fluoresce. Said linking moiety may include a hydrophilic or amphiphilic moiety of the kind described above, such as a C₂-C₃₀ polyethylene glycol moiety. This will help to ensure that the hydrophilicity of the chromophore is not impaired by the presence of said linking moiety.

Optionally, said aryl moiety R₁ may be further substituted by one or more hydrophilic substituents, such as hydroxy, which will serve to improve the hydrophilicity of said chromophore.

Said hydrophilic aryl moiety R₂ may comprise a phenyl ring, which phenyl ring may be substituted one or more times, preferably at least two times, by one or more hydrophilic substituents which serve to increase the hydrophilicity of said aryl moiety R₂. Said phenyl ring may preferably be linked by a single bond to the macrocyclic core of said chromophore or may alternatively be linked thereto by a C₁₋₆ branched or linear alkyl chain. Alternatively, said hydrophilic aryl moiety R₂ may comprise a heteroaryl ring, such as a pyridyl or quaternised pyridyl (pyridiniumyl) ring, which heteroaryl ring may be substituted one or more times, preferably at least two times, by one or more hydrophilic substituents which serve to increase the hydrophilicity of said aryl moiety R₂. Said heteroaryl ring may preferably be linked by a single bond to the macrocyclic core of said chromophore or may alternatively be linked thereto by a C₁₋₆ branched or linear alkyl chain. Said one or more hydrophilic substituents may advantageously be selected from hydroxy; alkoxy such as methoxy or ethoxy; C₂-C₁₅ polyethylene glycol; quatenised pyridyl (pyridiniumyl) such as N-methylpyridiniumyl; mono-, di- or poly-saccharide; C₁₋₆alkylsulfonate; a phosphonium group R₄P(R₅)(R₆)(R₇), wherein R₄ is a single bond or C₁₋₆ alkyl, and each of R₅, R₆ and R₇ is independently selected from hydrogen, an aryl ring such as a phenyl ring, a heteroaryl ring such as a pyridyl ring, and a C₁₋₆ alkyl chain, which aryl ring, heteroaryl ring or C₁₋₆ alkyl chain is unsubstituted or is substituted one or more times by hydroxy, C₁₋₆ alkyl or alkoxy, aryl, oxo, halogen, nitro, amino or cyano; or a phosphate or phosphonate group R₈OP(O)(OR₉)(OR₁₀) or R₈P(O)(OR₉)(OR₁₀) respectively, wherein R₈ is a single bond or C₁₋₆ alkyl and each of R₉ and R₁₀ is independently selected from hydrogen and C₁₋₆ alkyl. Preferably, each of said R₅, R₆ and R₇ may be the same, and may advantageously be unsubstituted phenyl. Suitably, said R₈ may be methyl. Advantageously, said R₉ and said R₁₀ may be the same, and/or may be methyl or ethyl.

In especially preferred embodiments, said hydrophilic aryl moiety R₂ is selected from m,m-(dihydroxy)phenyl

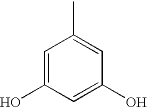

or a PEGylated derivative thereof; m,m,p-(trihydroxy)phenyl

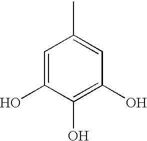

or a PEGylated derivative thereof; o,p,o-(trihydroxy)phenyl

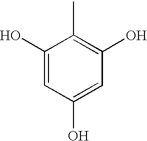

or a PEGylated derivative thereof; m- or p-((C₁₋₆)alkyltriphenylphosphonium)phenyl such as p-(methyltriphenylphosphonium)phenyl

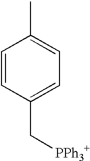

m- or p-(C₁₋₆alkylphosphono-di-alkoxy)phenyl such as p-methylphosphono-di-ethoxy)phenyl

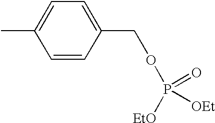

m- or p-(C₁₋₆alkylphosphonato-di-alkoxy)phenyl such as p-methylphosphonato-di-ethoxy)phenyl

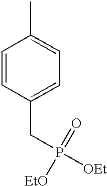

m- or p-(N-methyl-pyridiniumyl)phenyl

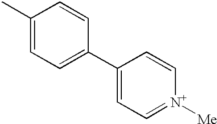

and meta- or para- sugar-substituted phenyl such as pentose-, hexose- or disaccharide-substituted phenyl

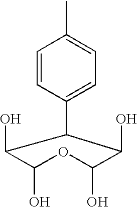

In other preferred embodiments, said hydrophilic aryl moiety R₂ comprises a quaternised pyridyl (pyridiniumyl) group such as a p-N—(C₁₋₆alkyl)pyridiniumyl group or m-N—(C₁₋₆alkyl)pyridiniumyl group. Quaternised pyridyl (pyridiniumyl) groups are highly hydrophilic and display advantageous properties when incorporated into chromophores in accordance with the invention. Particularly preferred groups in this regard are m- or p-N—((C₁₋₆)alkyl)pyridiniumyl, such as m-N-methylpyridiniumyl

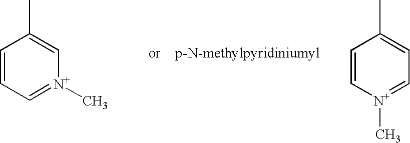

In other especially preferred embodiments, said quaternised pyridiniumyl group may comprise a zwitterionic group, such as p-N—(C₁₋₆alkylsulfonate)pyridiniumyl or m-N—(C₁₋₆alkylsulfonate)pyridiniumyl; in particular, p-N-(propylsulfonate)pyridiniumyl

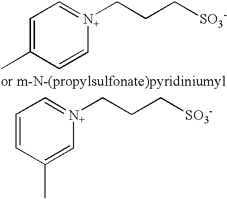

Preferably, the or each quaternised pyridiniumyl group R₂ may be associated with a halide counterion, such as an iodide counterion or, in most preferred embodiments, a chloride counterion.

In some advantageous embodiments, R₃ is H, such that said chromophore constitutes a 5,15-diaryl-porphyrin, -chlorin or -bacterlochlorin. In other advantageous embodiments, said R₃ is a hydrophilic aryl or non-aromatic moiety. For example, said R₃ may comprise a hydrophilic aryl moiety as defined above in relation to R₂. Said hydrophilic aryl moiety R₃ may be the same as said hydrophilic aryl moiety R₂, such that the chromophore possesses the same substituents at the 10, 15 and 20 positions thereof; or may be different from said hydrophilic aryl moiety R₂. Alternatively, said R₃ may comprise a hydrophilic alkyl moiety, such as a C₁₋₆ alkyl chain which is substituted one or more times by one or more hydrophilic substituents such as hydroxy or C₂₋₁₅ polyethylene glycol. In particularly preferred embodiments, said R₃ comprises polyhydroxy(C₁₋₆ alkyl), such as 1,2-dihydroxyethyl.

Chromophores in accordance with the invention wherein R₂ is the same as R₃ may be synthesised in accordance with methods known in the art, for example by acid catalysed condensation of benzaldehydes with pyrrole, or by means of the “MacDonald 2+2” method for synthesising porphyrins from dipyrromethanes (Arsenault et al, J. Chem. Soc. 1960, 82:4384-4389-incorporated herein by reference).

A generalised scheme for the synthesis of 5-isothiocyanatophenyl-15—pyridinium porphyrins, chlorins and bacteriochlorins in accordance with the present invention is set out as Scheme 1 below, in which “RX” represents a quaternising group such as C₁₋₆ alkyl or a hydrophilic substituent as defined above in relation to formulas (I) to (VII):

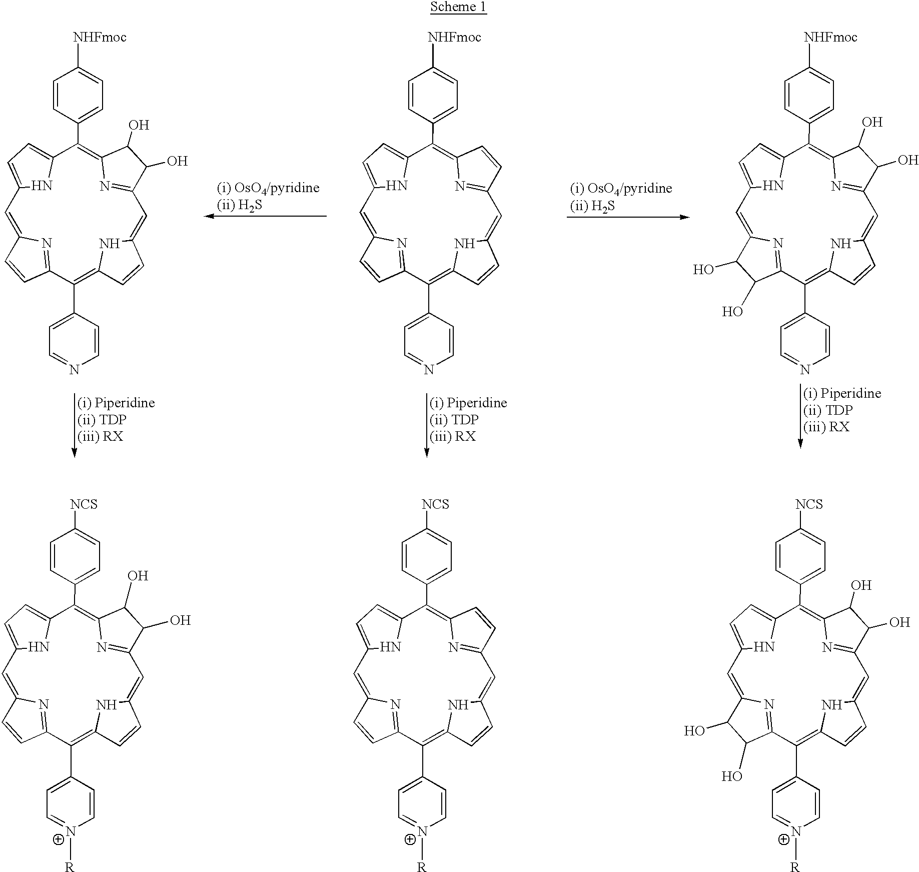

A generalised scheme for the synthesis of 5-isothiocyanatophenyl-15-methylphosphoniumphenyl porphyrins, chlorins and bacteriochlorins in accordance with the present invention is set out as Scheme 2 below, wherein R represents hydrogen, C₁₋₆ alkyl, a heterocyclic group or an aromatic group:

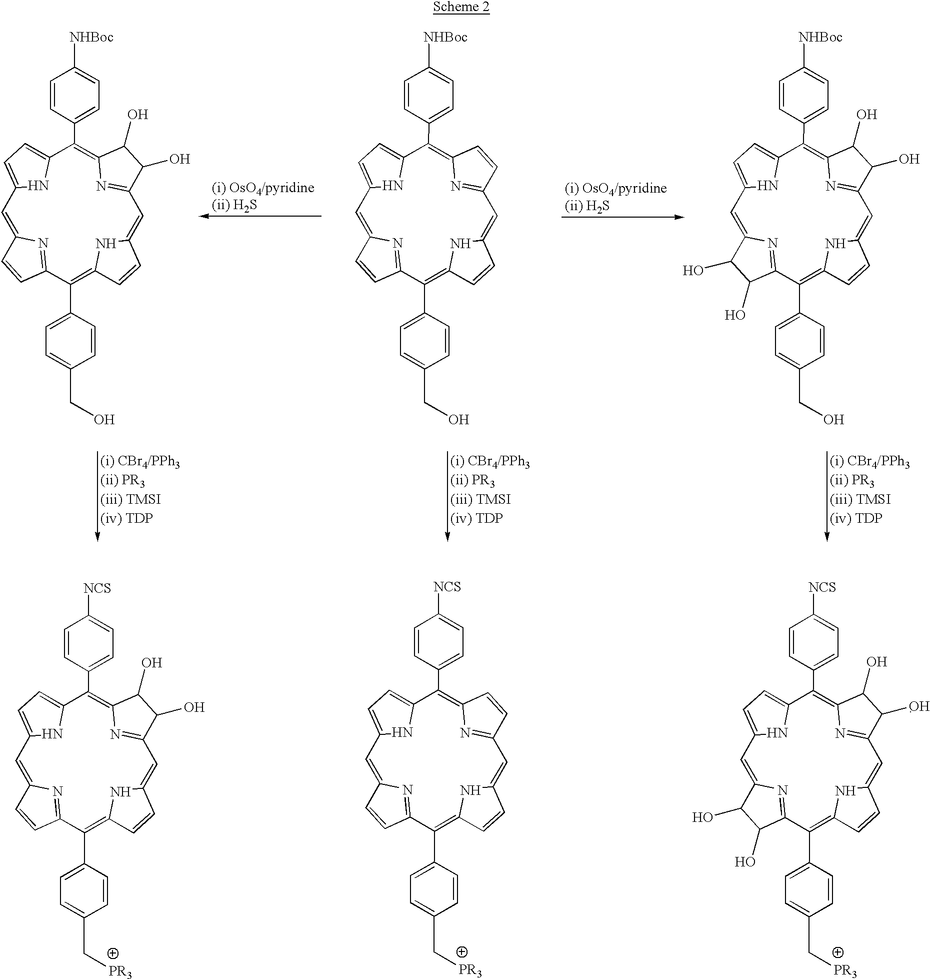

Porphyrin, chlorin and bacteriochlorin chromophores in accordance with the present invention wherein said R₂ and optionally said R₃ comprises pyridiniumylphenyl may be synthesised in accordance with the generalised reaction scheme set out below as Scheme 3, wherein “R” represents hydrogen or one or more hydrophilic substituents as defined above in relation to formulas (I) to (VII):

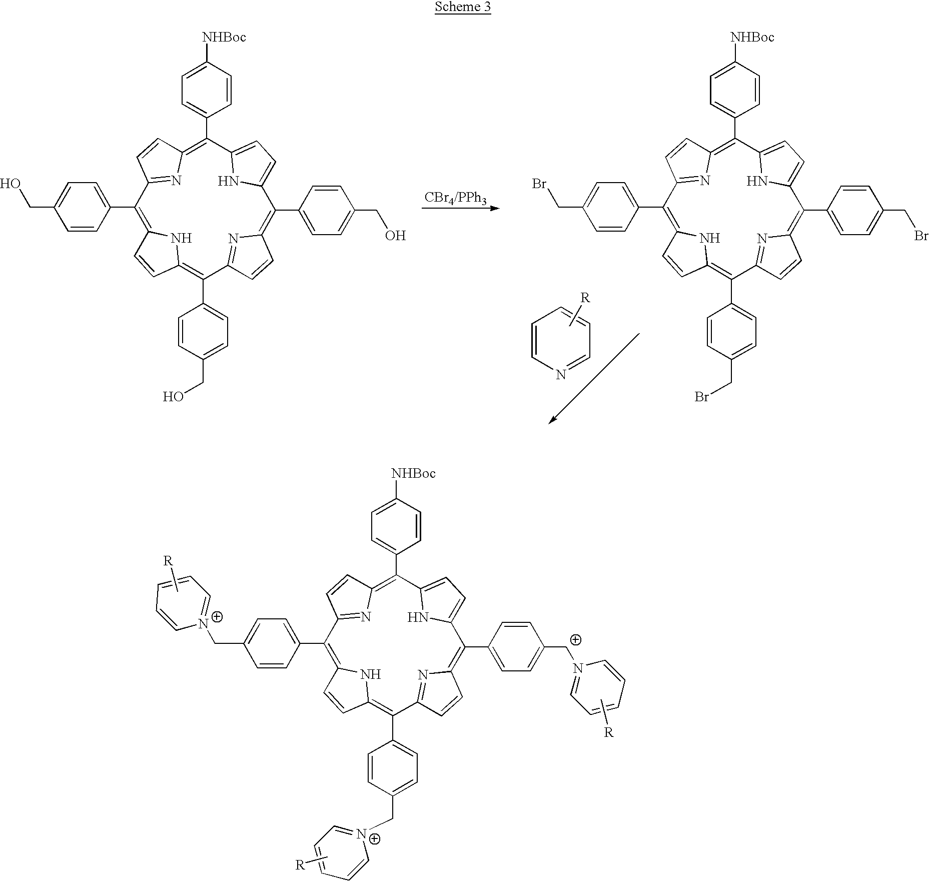

Porphyrin, chlorin and bacterlochlorin chromophores in accordance with the present invention wherein said R₂ and optionally said R₃ comprise alkylphosphonatophenyl or alkylphosphonophenyl may be synthesised in accordance with the ceneralised reaction scheme set out below as Scheme 4, wherein “R” represents OH, ONa, or O(C₁₋₆ alkyl):

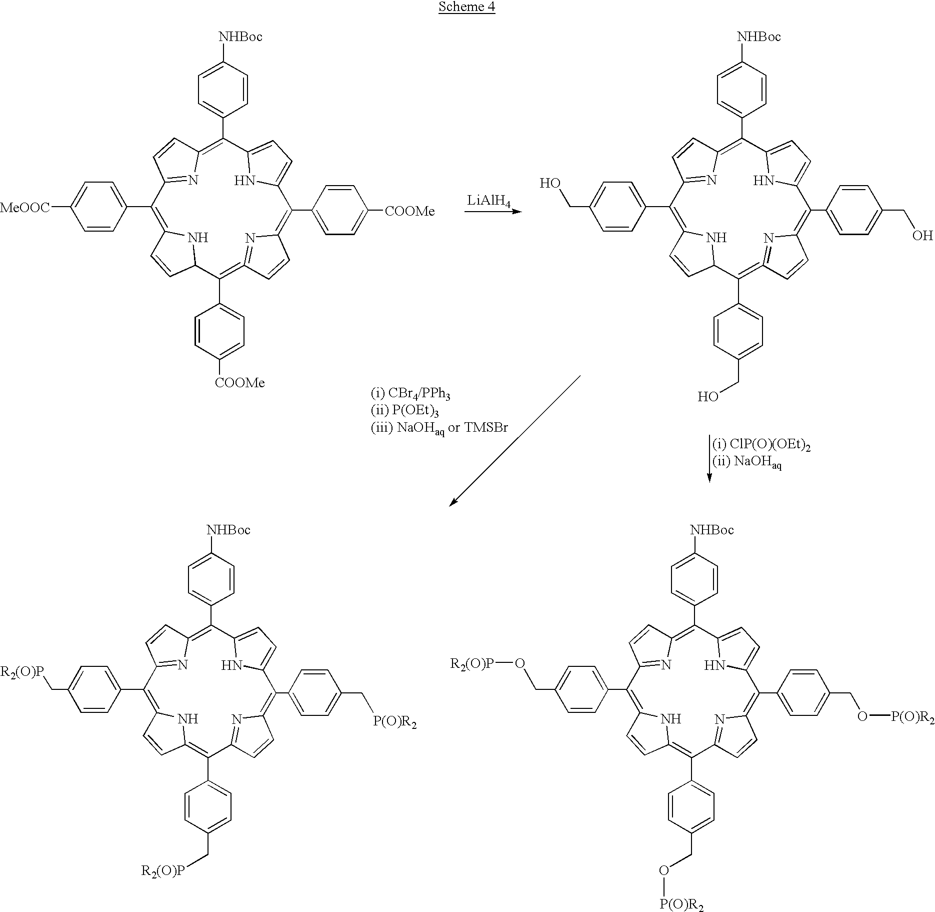

In a further aspect of the invention, there is provided a novel method for the synthesis of a 5,10,15,20-tetra-meso-substituted porphyrin, chlorin or bacteriochlorin chromophore having selected substituents at the 5, 10, 15 and 20 meso-positions thereof; comprising the steps of providing a 5,15-di-meso-substituted porphyrin, chlorin or bacteriochlorin chromophore; attaching a leaving group Q to the 10 and 20 meso-positions of said chromophore, which leaving group Q is selected from halide and triflate; providing a coupling reagent (R₁₁O)(R₁₂O)BR₁₃, wherein R₁₁ and R₁₂ are independently selected from H or C₁₋₆ alkyl, or R₁₁ and R₁₂ together constitute a C₁₋₆ alkyl chain bridging said two O atoms, and R₁₃ is vinyl or aryl, such as a hydrophilic aryl moiety as hereinbefore defined in relation to R₃; and reacting said chromophore with said coupling reagent in the presence of a base selected from potassium phosphate, sodium phosphate, caesium carbonate and barium hydroxide, and a Pd₀ catalyst; such that said R₁₃ replaces said leaving group Q at the 10- and 20-meso positions of said chromophore.

Pd₀-catalysed Suzuki coupling reactions using boronic acid or boronic ester reagents are known in the art, and are described for example in Miyaura & Suzuki, Palladium-catalyzed cross-coupling reactions of organoboron compounds, Chem. Rev. (1995) 95:2457-2483; the disclosure of which is incorporated herein by reference. Hitherto, however, attempts to carry out Suzuki-coupling at the meso-positions of porphyrins, chlorins or bacteriochlorins, as a means of importing selected substituents onto said meso-positions, have failed. The present inventors have found however that under the reaction conditions of the invention, Suzuki-coupling proceeds rapidly and successfully at the 10- and 20-meso-positions of the starting porphyrin, chlorin or bacteriochlorin chromophore. This method thereby enables convenient synthesis of tetra-meso-substituted porphyrins, chlorins or bacteriochlorins by Suzuki-coupling.

Said leaving group Q may be chloride, bromide, iodide or triflate (trifluoromethanesulfonate). Suitably, said leaving group Q may be bromide. Methods for the meso-bromination of di-meso-substituted porphrins, chlorins or bacteriochlorins are known in the art. For example, said 5,15-di-meso-substituted porphyrin, chlorin or bacteriochlorin chromophore may be halogenated at the 10- and 20-meso-positions thereof by way of reaction with halosuccinimide, such as bromosuccinimide.

Said coupling reagent may comprise a boronic ester or a boronic acid. In preferred embodiments, each of said R₁₁ and R₁₂ is H, such that said coupling reagent is a boronic acid.

Advantageously, said 5,15-di-meso-substituted porphyrin, chlorin or bacteriochlorin chromophore is a chromophore in accordance with the invention, or a protected form thereof. Thus, said 5,15-di-meso-substituted porphyrin, chlorin or bacteriochlorin chromophore may be selected from a porphyrin chromophore of formula (VIII) below:

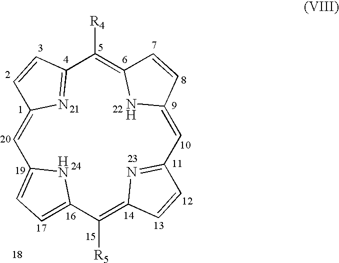

or a chlorin chromophore of any of formulas (IX), (X), (XI). amd (XII) below:

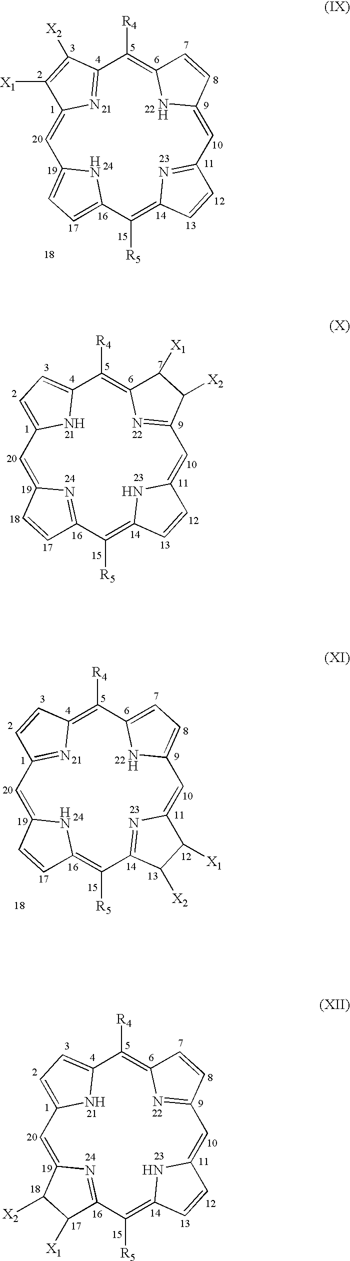

or a bacteriochlorin chromophore of any of formulas (XIII) and (XIV) below:

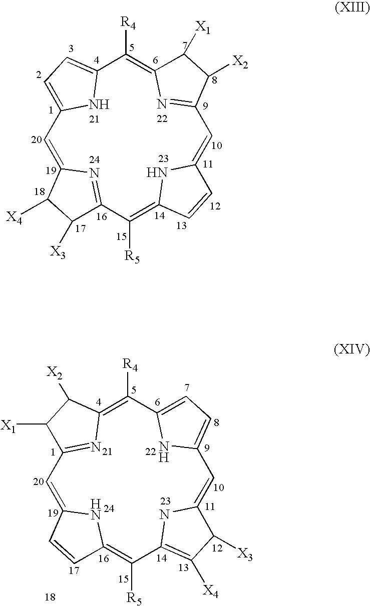

wherein R₄ is a group R₁ as defined above in relation to formulas (I) to (VII) or a protected form thereof or a group convertible thereto; R₅ is a group R₂ as defined above in relation to formulas (I) to (VII) or a protected form thereof or a group convertible thereto; and each of X₁, X₂, X₃ and X₄ is independently selected from H, OH, halogen, C₁₋₃ alkyl and OC₁₋₃ alkyl, or X₁ and X₂ and/or X₃ and X₄ together form a bridging moiety selected from O, CH₂, CH C₁₋₃ alkyl, or C(C₁₋₃ alkyl)₂, such that X₁ and X₂ and/or X₃ and X₄ with the adjacent C—C bond form an epoxide or cyclopropanyl structure.

Accordingly, where R₁₃ is a hydrophilic aryl substituent as defined above in relation to R₃, said 5,10,15,20-tetra-meso-substituted porphyrin, chlorin or bacteriochlorin chromophore may also constitute a chromophore in accordance with the present invention.

Said Pd₀ catalyst may, for example, comprise Pd(PPh₃)₄, PdCl₂(PPh₃)₂, or Pd(OAc)₂. Advantageously, said Pd₀ catalyst may comprise Pd(PPh₃)₄.

Said coupling reaction is performed in a solvent, which may be selected from toluene or dry THF. It is found that the coupling reaction proceeds swiftly in dry THF, and so dry THF is preferred as solvent.

Optionally, where said R₁₃ is vinyl, said 5,10,15,20-tetra-meso-substituted porphyrin, chlorin or bacteriochlorin chromophore may be subjected following said coupling reaction to an osmylation reaction utilising OsO₄, such as to convert said 10- and 20-vinyl substituents to hydroxyalkyl. Said osmylation reaction may be carried out under conditions identical to those suitable for converting a porphyrin to a di-beta-hydroxy-chlorin and then to a tetra-beta-hydroxy-bacteriochlorin. It is noted that this step may be performed in accordance with the invention on 5,10(vinyl), 15,20(vinyl)-meso-substituted porphyrin, chlorin or bacteriochlorin chromophores which are obtained otherwise than in accordance with the method of the invention, such as by way of Pd-catalysed Stitle coupling performed on said 5,15-di-meso-substituted chromophore in accordance with the method described in DiMagno et al, J. Org. Chem. 1993:58, 5983-5993, (incorporated herein by reference) wherein vinyl tributyl tin is used as a coupling reagent.

Where said tetra-meso-substituted chromophore is a porphyrin or a chlorin chromophore, said chromophore may be respectively converted to a chlorin or bacteriochlorin chromophore or to a bacteriochlorin chromophore in accordance with methods known to the man skilled in the art. For example, said porphyrin or chlorin chromophore may be osmylated by way of reaction with OsO₄, such as to produce a di-beta-hydroxy-chlorin or a tetra-beta-hydroxy-bacteriochlorin.

Generalised schemes for reactions in accordance with the present invention are set out in Schemes 5 and 6 below. In Scheme 5, “R” and “R₁” each represents one or more hydrophilic substituents as defined above in relation to R₂ and R₃ respectively. In Scheme 6, “R” represents one or more hydrophilic substituents as defined above in relation to R₂, and “X” represents a carbon or nitrogen atom.

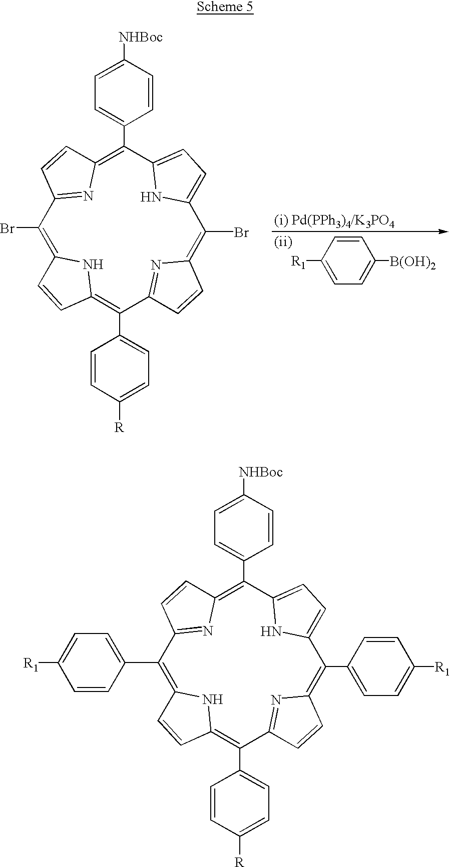

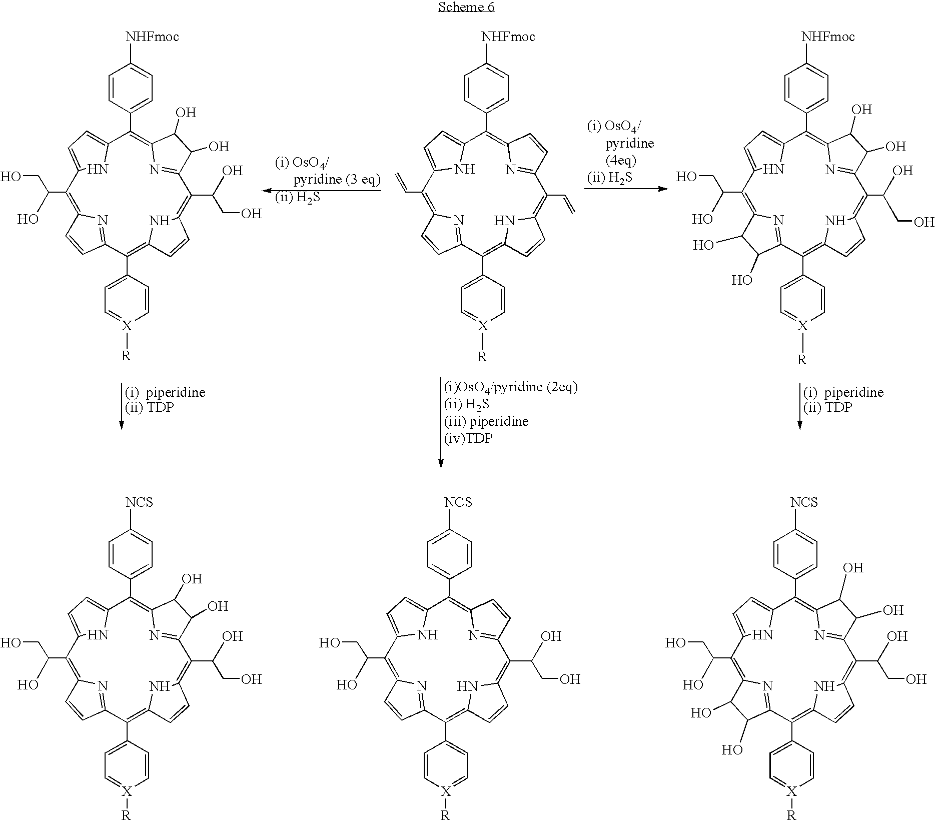

According to another aspect of the present invention, there is provided a 5,15-diphenylporphyrin, 5,15-diphenylchlorin or 5,15-diphenylbacteriochlorin chromophore, wherein each of the ortho-, meta, and/or para-positions of each of the 5- and 15-phenyl groups is substituted by a substituent P1-P5 and Q₁-Q₁ respectively which is independently H or an inert substituent which in combination with the other substituents P₁-P₅ and Q₁-Q₅ does not substantially impair the fluorescent properties of the chromophore, and the chromophore further comprises a conjugating group Z which is capable of conjugating the chromophore to a polypeptide molecule for delivering said chromophore to a specific biological target in vitro or in vivo.

Such chromophores are novel, and are each capable on excitation of emitting, fluorescent light at different and substantially non-overlapping wavelengths. As indicated above, the provision of conjugating group Z enables a chromophore in accordance with the invention to be specifically targetted to a specific biological target, thus facilitating control over the localisation of the chromophore in vitro or in vivo. Chromophores in accordance with the invention may therefore be usefully employed in fluorescence analysis and imaging applications (including FACS), or in PDT.

Advantageously, said fluorochrome is selected from the following compounds.

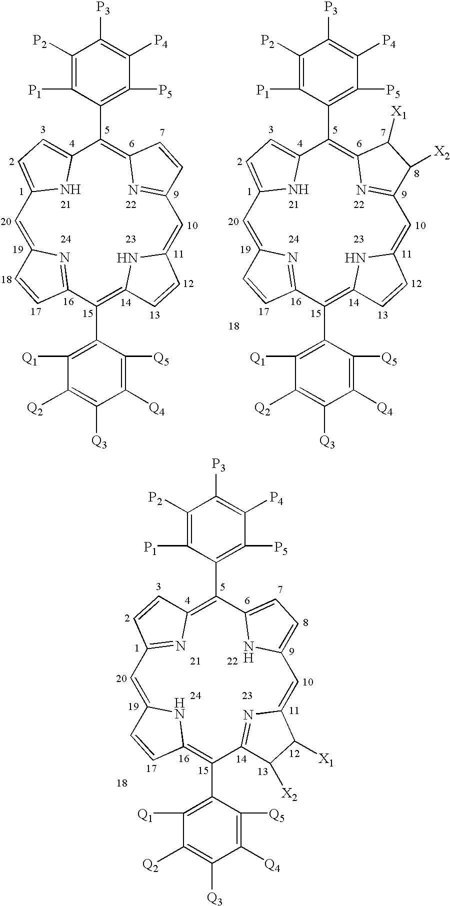

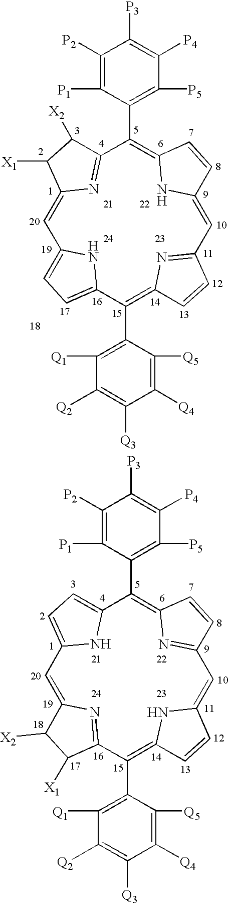

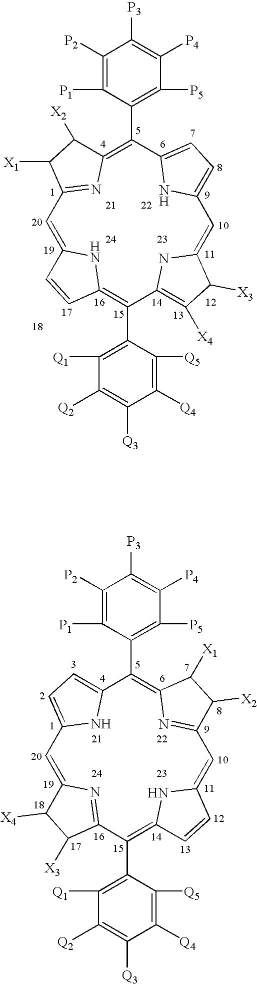

wherein each of X₁, X₂, X₃ and X₄ are as defined above in relation to the first aspect of the invention. Optionally, said chromophore may be further substituted at, one or more of the 2, 3, 7, 8, 12, 13, 17 or 18 positions thereof by a C₁₋₃ alkyl substituent.

In the foregoing chemical structures, Z has been omitted for clarity. However, said Z substituent may be attached to any of the 1-4,6-14, or 16-20 positions of each chromophore, or may be one of the substituents P₁-P₅ or Q₁-Q₅, or may be attached to one of the 5- or 15-phenyl groups through one of said substituents P₁-P₅ or Q₁-Q₅.

In some embodiments, each of P₁-P₅ is the same or substantially the same as the corresponding one of Q₁-Q₅, such that said two primary phenyl rings are symmetrically substituted. In other embodiments, one or more of P₁-P₅ is not the same as the corresponding one of Q₁-Q₅, such that said two primary phenyl rings are not symmetrically substituted. In particular, all of P₁-P₅ and/or all of Q₁-Q₅ may comprise H, such that one or both of said two primary phenyl rings is or are unsubstituted.

Advantageously, said substituents P₁-P₅ and Q₁-Q₅ collectively provide a degree of steric hindrance around the core of said chromophore which is sufficient to reduce the rate of spontaneous oxidation of said chromophore, such that said chromophore is substantially inert in air, but which does not to a substantial extent inhibit selective addition or substitution at the 2, 3, 7, 8, 12, 13, 17 or 18 positions around the core of said chromophore. Thus, each of P₁, P₅, Q₁ and Q₅ may be H. Typically, the total cumulative molecular weight of said substituents P₁-P₅ does not exceed 1000 gmol⁻¹, and the total cumulative molecular weight of said substituents Q₁-Q₅ does not exceed 1000 gmol⁻¹.

One or more of said substituents P₁-P₅ and Q₁-Q₅ may comprise —OH, —CN, —NO₂, halogen, -T or —OT, where T is a C₁-C₁₅ alkyl, cycloalkyl or aryl group or a hydroxylated, halogenated, sulphated or aminated derivative thereof or a carboxylic acid, ester, ether, polyether, amide, aldehyde or ketone derivative thereof One or more of said substituents P₁-P₅ and Q₁-Q₅ may additionally or alternatively comprise a C₃-C₁₂ cycloalkyl and/or aryl ring structures, or between two and six, preferably two-three, fused or linked C₃-C₁₂ cycloalkyl and/or aryl ring structures, each of which ring structures may optionally comprise one or more N, O or S atoms. In particular, one or more of said substituents P1-P5 and Q₁-Q₅ may comprise a quatenised amine or pyridyl group, such as an N-methyl pyridyl (pyridiniumyl) group.

Preferably, one of P₁-P₅ and Q₁-Q₅ is a conjugating substituent which comprises said conjugating group Z. In particularly preferred embodiments, said conjugating substituent is P₃ or Q₃, such that said conjugating group Z is provided on the para-position of one of the two primary phenyl rings.

Suitably, said conjugating group is as defined above in relation to the first aspect of the invention.

In particular, one or more of said substituents P₁-P₅ and Q₁-Q₅, not being said conjugating substituent, may consist of a member independently selected from the group consisting of A₁Z₁A₁₄; wherein Z₁ is Z₂, Z₂A₅ or Z₂A₅A6; A₁ and A₅ are independently selected from —(CA₂A₃)ₙ—, —C(Y)(CA₂A₃)ₙ—, —C(Y)Y′(CA₂A₃)ₙ—, —C(Y)NA₄(CA₂A₃)ₙ—, —NA₄C(Y)(CA₂A₃)ₙ—, —NA₄(CA₂A₃)ₙ, —YC(Y′)(CA₂A₃)ₙ— and —Y(CA₂A₃)ₙ—; n=0−6; Y and Y′ are independently O or S; A₂, A₃ and A₄ are independently H or C₁₋₂ alkyl which is unsubstituted or substituted by one or more fluorines; A₆=—(C₁₂H₄O)ₘ— or —S(O)ₚ; m=1−12; p=0−2; Z₂ is a single bond or Z₃; Z₃ is selected from Z₄, Z₅ and Z₆, wherein Z₃ is unsubstituted or substituted one or more times by OH, halo, CN, NO₂, A₁A₁₀, A₆A₈, NA₁₀A₁₁, C(Y)A₇, C(Y)Y′A₇, Y(CH₂)_{q}Y′A₇, Y(CH₂)_{q}A₇, C(Y)NA₁₀A₁₁, Y(CH₂)_{q}C(Y′)NA₁₀A₁₁, Y(CH₂)_{q}C(Y′)A₉, NA₁₀C(Y)NA₁₀A₁₁, NA₁₀C(Y)A₁₁, NA₁₀C(Y)Y′A₉, NA₁₀C(Y)Z₆, C(NA₁₀)NA₁₀A₁₁, C(NCN)NA₁₀A₁₁, C(NCN)SA₉, NA₁₀C(NCN)SA₉, NA₁₀C(NCN)NA₁₀A₁₁, NA₁₀S(O)₂A₉, S(O)ᵣA₉, NA₁₀C(Y)C(Y′)NA₁₀A₁₁, NA₁₀C(Y)C(Y′)A₁₀ or Z₆; q=0, 1 or 2; r=0−2; A₇ is independently selected from H and A₉, A₈ is O or A₉, A₉ is C₁₋₄ alkyl which is unsubstituted or substituted by one or more fluorines; A₁₀ is OA₉ or A₁₁; A₁₁ is A₇ or when A₁₀ and A₁₁ are as NA₁₀A₁₁ they may together with the nitrogen form a 5 to 7 membered ring comprising only carbon atoms or carbon atoms and at least one heteroatom selected from O, N and S; Z₄ is C₆₋₁₂ aryl or aryloxyC₁₋₃alkyl; Z₅ is selected from furanyl, tetrahydrofuranyl, indanyl, indenyl, tetrahydropyranyl, pyranyl, thiopyranyl, tetrahydrothiopyranyl, tetrahydrothienyl, thienyl, C₃₋₈ cycloalkyl or C₄₋₈ cycloalkyl containing one or two unsaturated bonds, and C₇₋₁₁ polycycloalkyl; Z₆ is selected from N-azolyl, dioxadiazinyl, dioxadiazolyl, dioxanyl, 2-N-dioxatriazinyl, dioxazinyl, N-dioxazolyl, dioxolyl, dithiadiazinyl, dithiadiazolyl, N-dithiatriazinyl, dithiazinyl, N-dithiazolyl, 1-N-imidazolyl, N-morpholinyl, pyrollyl, tetrazolyl, thiazolyl, triazolyl, oxazinyl, oxazolyl, naphthydrinyl, oxadiazinyl, oxadiazolyl, oxatetrazinyl, oxatriazinyl, oxatriazolyl, oxazinyl, oxazolyl, pentazinyl, phthalazinyl, N-piperidinyl, N,N-piperazinyl, 1-N-pyrazolyl, pyridazinyl, pyridinyl, pyrimidinyl, tetrathiazinyl, tetrazinyl, 1-N-tetrazolyl, tetroxazinyl, thiadiazinyl, thiadiazoyl, thiatetrazinyl, thiatriazinyl, thiatriazolyl, thiazolyl, triazinyl, 1-N-triazolyl, trioxadazinyl, trioxanyl, trioxazinyl, trioxazolyl, trithiadiazinyl, trithiazinyl, trithiadiazolyl; wherein Z₄, Z₅ or Z₆ may be fused to one or more other members selected independently from Z₄, Z₅ and Z₆; A₁₄ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl or heterocyclic moiety is unsubstituted or substituted by 1 to 3 methyl groups, one ethyl group, or a hydroxyl group.

Said conjugating substituent may consist of a member selected from the group consisting of A₁Z₁Z; wherein Z₁ is Z₂, Z₂A₅ or Z₂A₅A₆; A₁ and A₅ are independently selected from —(CA₂A₃)ₙ—, —C(Y)(CA₂A₃)ₙ—, —C(Y)Y′(CA₂A₃)ₙ—, —C(Y)NA₄(CA₂A₃)ₙ—, —NA₄C(Y)(CA₂A₃)ₙ—, —NA₄(CA₂A₃)ₙ, —YC(Y′)(CA₂A₃)ₙ— and Y(CA₂A₃)ₙ—; n=0−6; Y and Y′ are independently O or S; A₂, A₃ and A₄ are independently H or C₁₋₂ alkyl which is unsubstituted or substituted by one or more fluorines; A₆=—(C₂H⁴O)ₘ— or —S(O)ₚ; m=1−12; p=0-2; Z₂ is a single bond or Z₃; Z₃ is selected from Z₄, Z₅ and Z₆, wherein Z₃ is unsubstituted or substituted one or more times by OH, halo, CN, NO₂, A₁A₁₀, A₆A₈, NA₁₀A₁₁, C(Y)A₇, C(Y)Y′A₇, Y(CH₂)_{q}Y′A₇, Y(CH₂)_{q}A₇, C(Y)NA₁₀A₁₁, Y(CH₂)_{q}C(Y′)NA₁₀A₁₁, Y(CH₂)_{q}C(Y′)A₉, NA₁₀C(Y)NA₁₀A₁₁, NA₁₀C(Y)A₁₁, NA₁₀C(Y)Y′A₉, NA₁₀C(Y)Z₆, C(NA₁₀)NA₁₀A₁₁, C(NCN)NA₁₀A₁₁, C(NCN)SA₉, NA₁₀C(NCN)SA₉, NA₁₀C(NCN)NA₁₀A₁₁, NA₁₀S(O)₂A₉, S(O)ᵣA₉, NA₁₀C(Y)C(Y′)NA₁₀A₁₁, NA₁₀C(Y)C(Y′)A₁₀ or Z₆; q=0, 1 or 2; r=0−2; A₇ is independently selected from H and A₉; A₈ is O or A₉; A₉ is C₁₋₄ alkyl which is unsubstituted or substituted by one or more fluorines; A₁₀ is OA₉ or A₁₁; A₁₁ is A₇ or when A₁₀ and A₁₁ are as NA₁₀A₁₁ they may together with the nitrogen form a 5 to 7 membered ring comprising only carbon atoms or carbon atoms and at least one heteroatom selected from O, N and S; Z₄ is C₆₋₁₂ aryl or aryloxyC₁₋₃alkyl; Z₅ is selected from furanyl, tetrahydrofuranyl, indanyl, indenyl, tetrahydropyranyl, pyranyl, thiopyranyl, tetrahydrothiopyranyl, tetrahydrothienyl, thienyl, C₃₋₈ cycloalkyl or C₄₋₈ cycloalkyl containing one or two unsaturated bonds, and C₇₋₁₁ polycycloalkyl; Z₆ is selected from Nazolyl, dioxadiazinyl, dioxadiazolyl, dioxanyl, 2-N-dioxatriazinyl, dioxazinyl, N-dioxazolyl, dioxolyl, dithiadiazinyl, dithiadiazolyl, N-dithiatriazinyl, dithiazinyl, N-dithiazolyl, 1-N-imidazolyl, N-morpholinyl, pyrollyl, tetrazolyl, thiazolyl, triazolyl, oxazinyl, oxazolyl, naphthydrinyl, oxadiazinyt, oxadiazolyl, oxatetrazinyl, oxatriazinyl, oxatriazolyl, oxazinyl, oxazolyl, pentazinyl, phthalazinyl, N-piperidinyl, N,N-piperazinyl, 1-N-pyrazolyl, pyridazinyl, pyridinyt, pyrimidinyl, tetrathiazinyt, tetrazinyl, 1-N-tetrazolyl, tetroxazinyl, thiadiazinyl, thiadiazoyl, thiatetrazinyl, thiatriazinyl, thiatriazolyl, thiazolyl, triazinyl, 1-N-triazolyl, trioxadazinyl, trioxanyl, trioxazinyl, trioxazolyl, trithiadiazinyl, trithiazinyl, trithiadiazolyl; wherein Z₄, Z₅ or Z₆ may be fused to one or more other members selected independently from Z₄, Z₅ and Z₆.

In particular embodiments of the present invention, said chromophore may comprise a chromophore having a structure set out as (x), (y) or (z) below:

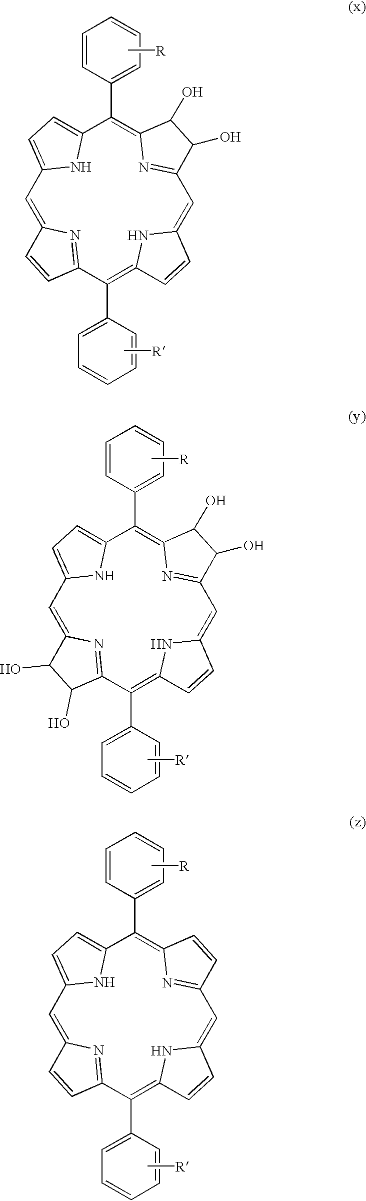

wherein R and R′ may be any of the following combinations:

| R | R′ |
|---|---|
| 4-H | 4-NCS |
| 4-Me | 4-NCS |
| 4-Br | 4-NCS |
| 4-CO₂Me | 4-NCS |
| 3,4,5-tris(OMe) | 4-NCS |
| 4-NCS | 4-OMe |
| 4-NCS | 4-Me |
| 4-NCS | 4-CO₂Me |
| 4-NCS | 4-Br |
| 4-NCS | 4-CN |
| 4-NCS | 4-CO₂Me |

In another embodiment of the present invention, said chromophore may comprise a porphyrin chromophore having the structure set out below:

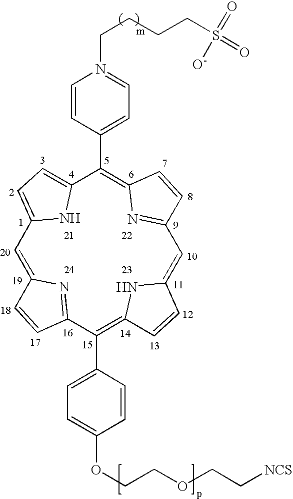

wherein m=0−6; p=0−15, preferably 0−5; or the corresponding chlorin or bacteriochlorin chromophore.

According to another aspect of the present invention, there is provided a set of fluorochromic markers for multicolour fluorochromic analysis, comprising at least two chromophores selected from the group consisting of a porphyrin chromophore, a chlorin chromophore and a bacteriochlorin chromophore, each of which chromophores comprises the same porphyrin skeleton, each of which chromophores comprises one or more substituents on said porphyrin skeleton, one of which substituents is a conjugating substituent L comprising a conjugating group Z, wherein Z is a conjugating group capable of conjugating each of said chromophores to a polypeptide molecule for delivering each chromophore to one of a plurality of different specific biological targets.

Preferably, each of the other of said substituents on the skeleton is independently H or an inert substituent R which together with said conjugating substituent L and all of the other core substituents does not substantially impair the fluorescent properties of each chromophore.

It has been found that each of the chromophores in a set in accordance with the present invention, on excitation, will emit fluorescent light at a different discrete wavelength. Thus, all of the chromophores within the set can be excited by a single laser, producing separate emission bands which can be substantially individually resolved. Moreover, all of the chromophores provided in said set share substantially the same molecular structure, and will accordingly share substantially the same biochemical and physicochemical properties, including substantially the same degree of efficiency of bioconjugation to a biological target under given conditions. Accordingly, a set of chromophores in accordance with the present invention may be usefully employed in fluorescence analysis and sorting applications, including FACS, for the convenient sorting and analysis of several types of cells or other biological targets. The components of such a set may, for example, be introduced to a mixture comprising one or more of said different specific biological targets, under conditions which will allow the delivery of each chromophore to its respective specific biological target; and said mixture may be exposed to light so as to cause said chromophores to fluoresce. A multicolour analysis may then be carried out for identifying the different emission bands produced by each chromophore, thereby permitting counting and visualisation of the location of each of the different biological targets.

Said set of chromophores may in particular comprise two or more of a porphyrin chromophore in accordance with any aspect of the present invention, the corresponding chlorin chromophore, and the corresponding bacteriochlorin chromophore. (By “corresponding” herein is meant having the same meso-substituents around the macrocyclic core of the molecule).

In a chromophore in accordance with the present invention, or in each member of a chromophore set in accordance with the present invention, said conjugating group Z may be conjugated to a binding protein which is adapted to bind specifically to said biological target. Alternatively, said conjugating group Z may be conjugated to a bridging polypeptide which is adapted to bind to a complementary bridging polypeptide so as to couple said chromophore to said complementary bridging polypeptide.

In some embodiments, said bridging polypeptide may be bound to said complementary bridging polypeptide, and said complementary bridging polypeptide may comprise or be coupled to or fused with a binding protein which is adapted to bind specifically to said biological target. Accordingly, said chromophore may be covalently linked to said binding protein by means of said bridging polypeptide and said complementary bridging polypeptide.

According, to another aspect of the present invention, there is provided a kit comprising a chromophore in accordance with the present invention or a set of chromophores in accordance with the present invention, wherein said chromophore or each chromophore is conjugated to a bridging polypeptide that is adapted to bind to a complementary bridging polypeptide so as to couple the chromophore to said complementary bridging polypeptide; and a construct or plurality of constructs each of which comprises said complementary bridging polypeptide fused or coupled to a binding protein which is adapted to bind specifically to said biological target; the arrangement being such that said chromophore or each chromophore in the kit is adapted to bind to a different construct in the kit with specificity for said specific biological target, so as to link said or each chromophore to a binding protein with specificity for said specific biological target.

Said binding protein may, for example, be an antibody such as a monoclonal or polyclonal antibody or a fragment thereof with specificity for a target specific molecule on the surface of said biological target. In particular, said antibody may be a phage antibody, that is an antibody expressed on the surface of a bacteriophage. Alternatively said binding protein may be a protein which is adapted to bind to one or more cell surface molecules or receptors, such as a serum albumin protein. As yet a further alternative, said binding protein may comprise a low density lipoprotein, such as a fatty acid chain, which is adapted for insertion into a cell membrane. When conjugated to a chromophore, such a lipoprotein can serve to anchor the chromophore to a cell membrane.

Said bridging polypeptide may comprise calmodulin, and said complementary bridging polypeptide may comprise calmodulin binding peptide; or vice versa. Preferably, however, said bridging polypeptide may comprise avidin or streptavidin, and said complementary bridging polypeptide may comprise biotin; or vice versa. In particular, said or each chromophore in a kit in accordance with the present invention may be conjugated to avidin, and said or each construct may comprise a biotinylated monoclonal antibody with specificity for a target specific molecule on the surface of said biological target. Accordingly, when said avidin-linked chromophore is allowed to bind said biotinylated antibody, said chromophore will become firmly linked to said antibody. Conveniently, said or each biotinylated monoclonal antibody in the kit may be selected and/or readily substituted, so as to enable said or each chromophore to be delivered to any desired biological target. Methods for the preparation of monoclonal antibodies and for the biotinylation thereof are well known in the art.

According to another aspect of the present invention, there is provided a method for attaching a chromophore in accordance with the invention or a set of chromophores in accordance with the invention to said specific biological target or targets; comprising the steps of providing a kit in accordance with the present invention, and introducing the components of said kit into the vicinity of said specific biological target or targets, under conditions suitable for enabling the binding of said or each binding protein to said specific biological target or targets. Advantageously, the components of said kit may be allowed to associate with one another prior to introduction to said target or targets, so as to enable the bridging polypeptide conjugated to said or each chromophore to bind to a complementary bridging polypeptide provided on one of said constructs in the kit. This will ensure that said or each chromophore in the kit is linked to a binding protein prior to introduction of said chromophore to said target or targets. Alternatively, the components of said kit may be introduced sequentially to said target or targets.

Typically, said specific biological target may be a cell or a membrane. Said specific biological target may be in vivo or in vitro (ex vivo). Said biological target may, for example, be a cancer cell, a tumour cell, a cell infected with HIV or with any other microbe or virus, a cell responsible for detrimental activity in auto-immune disease, a foreign or diseased cell, or any other such cell.

In some embodiments of the present invention, said biological target is a cell in vitro, and said target specific molecule comprises a molecule exposed on the surface of said cell, such as a polypeptide, carbohydrate, fatty acid, lipoprotein, phospholipid or other biological molecule. Preferably, said target specific molecule is specifically expressed by, or is over-expressed by, said cell. Said target specific molecule may, for example, be a T cell marker such as CD4 or CD8. Accordingly, when a chromophore in accordance with the present invention or a chromophore forming part of a set of chromophores in accordance with the present invention is attached to said cell, and said cell is illuminated so as to cause fluorescence of said chromophore, the fluorescence of the chromophore will enable said cell to be visualised and counted and/or sorted by FACS.

According to a further aspect of the present invention, therefore, there is provided a method for fluorescence-activated sorting of target cells from a mixture of cells, comprising the step of attaching to said target cells a chromophore in accordance with the invention or a set of chromophores in accordance with the invention, illuminating said mixture of cells so as to cause fluorescence of one or more of said chromophores attached to said target cells, imparting a charge to the fluorescing cells, and passing said mixture of cells through a polarised environment so as to cause or allow said charged cells to be separated from said mixture.

According to another aspect of the present invention, there is provided a method for the visualisation and/or counting of a plurality of target cells, said target cells including cells of two or three different cell types, comprising the steps of providing a chromophore set in accordance with the present invention, which chromophore set comprises two or three chromophores each of which is adapted to be delivered to a different one of said cell types; attaching said chromophores in the set to said target cells in accordance with the method of the present invention; illuminating said target cells so as to cause the emission of fluorescence by said chromophores; detecting the fluorescent emission bands produced by each of said chromophores; and optionally measuring for each of said bands the area under an emission/wavelength curve, so as to obtain a measure of the number of fluorescent cells of each respective cell type.

In other embodiments of the present invention, said target cell is a cell in vivo, such as a cancer cell, tumour cell, or an infected, foreign or diseased cell, and said target specific molecule is a target cell specific molecule which is specifically expressed by, or is over-expressed by, or is attached to, and is exposed on, the surface of said target cell; such as a target cell specific membrane protein. Accordingly, when a chromophore in accordance with the invention is delivered to said target specific molecule, said chromophore will be caused to be attached to said cell. If said cell is subsequently illuminated with light at a wavelength suitable for causing the excitation of said chromophore, said chromophore attached to said cell may be caused to be excited, and this may result in the production of singlet oxygen in the immediate vicinity of said cell, hence bringing about the death of the cell.

In especially preferred embodiments, said target cell specific molecule comprises an internalisation receptor on the surface of said cell, which internalisation receptor is capable of binding said binding protein and thereby mediating the internalisation of said chromophore within said cell. Accordingly, subsequent illumination of said cell with light at a wavelength suitable for causing excitation of said chromophore may result in the production of singlet oxygen within said cell, hence bringing about the death of said cell.

The present invention therefore comprehends a method for causing the death of a target cell, comprising the step of attaching a chromophore in accordance with the present invention to said cell and illuminating said cell so as to cause the production of singlet oxygen in the vicinity of said cell, thereby causing the death of the cell. Preferably, said chromophore is attached to an internalisation receptor on the surface of said cell, which internalisation receptor is capable of mediating the internalisation of said chromophore within said cell, and said cell is thereafter illuminated such as to cause the production of singlet oxygen within said cell, thereby causing the death of the cell.

Preferably, where said chromophore is adapted to be internalised within the cell, said chromophore comprises a cationic group such as a quatenised amine or pyridyl (pyridiniumyl) group, or a phosphonium group, so as to promote the intracellular accumulation of said chromophore around the mitochondria of the cell, owing to the net negative charge on the mitochondrial membrane. This will result in the rapid and efficient killing of the cell, on production of singlet oxygen by decay of the chromophore.

In accordance with another aspect of the invention, there is provided a method for treating a disease or disorder which is characterised by the presence in the body of diseased or undesired cells, such as tumours, cancers, viral infections such as HIV infection, or autoimmune disorders such as rheumatoid arthritis or multiple sclerosis, comprising the step of administering to a patient in need thereof an effective amount of a chromophore in accordance with the invention, which chromophore is adapted to be targeted to a target cell specific molecule on the surface of said diseased or undesired cells for attachment thereto, such that the chromophore is caused to be attached to said cells, and illuminating said cells with light so as to cause the production of singlet oxygen in the vicinity of said cells, thereby killing said cells. Suitably, said target cell specific molecule comprises an internalisation receptor, and said chromophore is adapted to be internalised within said cells on delivery to said internalisation receptor, such as to enable the production of singlet oxygen within said cells on illumination thereof.

Said chromophore may be administered topically or systemically to said patient. For example, said chromophore may be administered by injection.

In accordance with yet another aspect of the invention, there is provided a pharmaceutical composition for administration to a patient for the treatment of a disease or disorder which is characterised by the presence in the body of diseased or undesired cells, such as tumours, cancers, viral infections such as HIV infection, or autoimmune disorders such as rheumatoid arthritis or multiple sclerosis, which composition comprises a chromophore in accordance with the present invention that is adapted to be delivered to said diseased or undesired cells, and a suitable carrier.

Yet another aspect of the invention envisages a chromophore in accordance with the invention for use in the production of a medicament, for use in the treatment of patients suffering from a disease or disorder which is characterised by the presence in the body of diseased or undesirable cells, such as tumours, cancers, viral infections including HIV infection, and autoimmune disorders including rheumatoid arthritis or multiple sclerosis; said chromophore being adapted for delivery to said diseased or undesired cells.

### DETAILED DESCRIPTION OF EXAMPLES OF THE INVENTION

Following are descriptions and examples, by way of illustration only, of embodiments of the invention and methods for putting the invention into effect.

Synthesis of Chromophores

Instrumentation and Materials

Melting points are uncorrected. ¹H/¹³C NMR spectra were recorded on Jeol JNM EX270 FT-NNMR spectrometer, and are referenced to tetramethylsilane unless otherwise stated. I.R. spectra were obtained using a series 1600 FT-I.R. and nominal mass spectra were obtained by Kratos Kompact MALDI II spectrometer. Accurate mass were obtained from EPSRC Mass Spectrometry Service, Swansea. The electronic spectra were obtained using Unicam UV-2 or UV-4 spectrometers and were taken in DCM unless otherwise stated. All reagents and solvents were commercially available and of reagent grade or higher, and were, unless otherwise specified, used as received. TLC analysis were performed on Merck silica-gel 60 plates (F254, 500 μm thickness). Merck Silica-Gel 60 (230-400 mesh) was used for flash chromatographic purification.

Descriptions

(1) 5-(4-Acetamidophenyl)-10,15,20-tris(3,5-dimethoxyphenyl)porphyrin

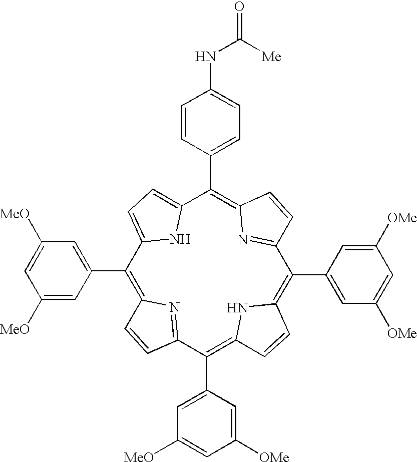

4-Acetamidobenzaldehyde (3.36 g, 0.02 mol) and 3,5-dimethoxybenzaldehyde (10 mL, 0.06 mol) were stirred in propionic acid (300 mL) at 90° C. Pyrrole (5.5 mL, 0.08 mol) was added and the mixture stirred under reflux for 30 min. Upon cooling the reaction mixture was evaporated in vacuo to yield a dark purple solid. The crude mixture of porphyrin isomers was purified by flash chromatography (silica, eluent: CH₂Cl₂/EtOAc, 4:1). Relevant fractions were combined, dried (Na₂SO₄) and evaporated it? vacuo to yield 1 as a purple solid (1.55 g, 9.1%); R_{f}=0.50 (silica, CH₂Cl₂/EtOAc, 4:1); mp>350° C. decomp; ¹H NM [270 MHz, CDCl₃] δ-2.96 (2H, br s, NH), 2.23 (3H, s, NHCOCH₃), 3.93 (18H, s, 3, 5-OCH₃), 6.99 (3H, s, 10, 15, 20-Ar-4-H), 7.07 (2H, n, J*=8 Hz, 5-Ar-3,5-H), 7.38 (6H, s, 10, 15, 20-Ar-2,6-H), 7.44 (2H, m, J*=8 Hz, 5-Ar-2,6-H), 8.86-8.93 (8H, m, β-H), 10.42 (1H, br s, NHCOCH₃); ¹³C NMR [67.5 MHz, CDCl₃] δ 20.4, 23.9, 103.9, 113.5, 117.3, 119, 119.4, 119.6, 120, 129, 131.1, 131.3, 131.4, 131.8, 134.5, 135.6, 136.8, 139.3, 143.1, 158.6, 160.3, 167.9, 168.7; UV-vis (CH₂Cl₂) λₘₐₓ 421, 515, 551, 590, 650 nm; MS (MALDI-TOF) m/z 852 (M⁺, 100%).

(2) 5-(4-Aminophenyl)-10,15,20-tris(3,5-dimethoxyphenyl)porphyrin

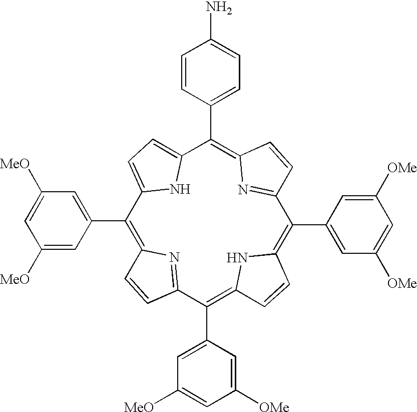

Porphyrin 1 (500 mg, 0.587 mmol) was dissolved in 18% HCl (100 mL) and the solution heated for 2 hours under reflux. Upon cooling the reaction mixture was evaporated in vacuo to yield a crude green solid. The solid was redissolved in a 9:1 mixture of dichloromethane/triethylamine (200 mL) and stirred for 10 min at room temperature. The solution was then washed with water (3×200 mL) and brine (200 mL), the organic layer separated and dried (Na₂SO₄). Excess solvent was evaporated in vacuo and the crude purple solid purified by flash chromatography (silica, eluent: CH₂Cl₂/EtOAc, 4:1). Relevant fractions were combined, dried (Na₂SO₄) and evaporated in vacuo to yield 2 as a purple solid (426 mg, 89.7%); R_{f}=0.89 (silica, CH₂Cl₂/EtOAc, 4:1); mp>350° C. decomp.; ¹H NMR [270 MHz, CDCl₃] δ-2.80 (2H, br s, NH), 3.96 (18H, s, 3, 5-OCH₃), 6.90 (3H, s, 10, 15, 20-Ar-4-H), 7.06 (2H, m, J*=8 Hz, 5-Ar-3, 5-H), 7.40 (6H, s, 10, 15, 20-Ar-2,6-H), 7.98 (2H, m, J*=8 Hz, 5-Ar-2,6-H), 8.93 (8H, m, β-H); ¹³C NMR [67.5 MHz, CDCl₃] δ 20.13, 100.6, 113.9, 114.3, 115.7, 119.8, 120.1, 121.4, 130.2, 131.4, 132.7, 136.1, 144.5, 144.6, 146.5, 159.3; UV-vis (CH₂Cl₂) λₘₐₓ 422, 517, 553, 593, 651 nm; MS (MALDI-TOF) m/z 809 (M⁺, 100%).

(3) 5-(4-Aminophenyl)-10,15,20-tris(3,5-dihydroxyphenyl)porphyrin

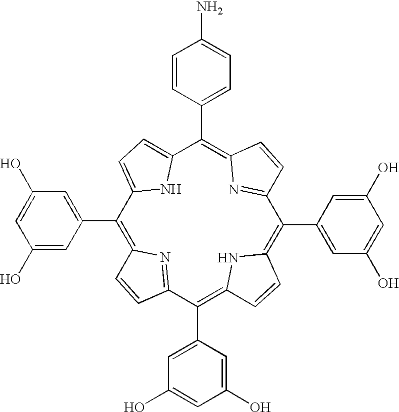

To a stirred solution of 2 (1 g, 1.23 mmol) in freshly distilled chloroform (50 mL) was added boron tribromide (1.17 mL, 0.012 mol). The reaction was allowed to proceed under argon for 17 hours at room temperature. The reaction was subsequently cooled to 0° C., water (20 mL) added and the solution stirred for a further 60 min. The reaction was evaporated to dryness in vacuo and redissolved in a 9:1 mixture of chloroform/triethylamine (500 mL). The solution was washed with water (3×500 mL) and brine (500 mL), the organic layer separated, dried (Na₂SO₄), and evaporated in vacuo to yield a crude purple solid. The crude solid was purified by flash chromatography (silica, eluent: CHCl₃/MeOH, 9:1). Relevant fractions were combined, dried (Na₂SO₄) and evaporated in vacuo to yield 3 as a purple solid (667 mg, 74.5%); R_{f}=0.19 (silica, CHCl₃/MeOH, 9:1); mp>350° C. decomp.; ¹H NMR [270 MHz, (CD₃)₂SO] δ-2.95 (211, br s, NH), 5.56 (2H, br s, NH₂), 6.69 (3H, s, 10, 15, 20-Ar-4-H), 7.02 (2H, m, J*=8 Hz, 5-Ar-3,5-H), 7.06 (611 s, 10, 15, 20-Ar-2,6-H), 7.87 (21, m, J*=8 Hz, 5-Ar-2,6-H), 8.94 (81, s, β-H), 9.75 (611, br s, 3,5-OH); ¹³C NMR [67.5 MHz, (CD₃)₂SO] β102.3, 112.5, 113.9, 114.1, 119.2, 119.7, 121.5, 127.5, 128.3, 130.7-131.3, 135.5, 142.8, 142.9, 148.6, 156.4, 156.5; UV-vis (CH₂Cl₂) λₘₐₓ 422, 517, 553, 592, 649 nm; MS (MALDI-TOF) m/z 726 (M⁺, 100%).

(4) 5-(4-Acetamidophenyl)-10,15,20-tris(4-pyridyl)porphyrin

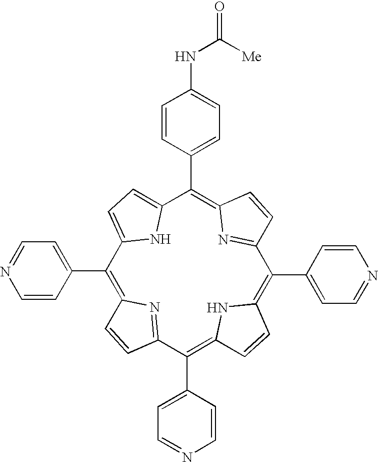

4-Acetamidobenzaldehyde (3.26 g, 0.02 mol) and 4-pyridinecarboxaldehyde (5.66 mL, 0.06 mol) were stirred in propionic acid (300 mL) at 90° C. Pyrrole (5.4 mL, 0.08 mol) was added and the mixture stirred under reflux for 30 min. Upon cooling the reaction mixture was evaporated in vacuo to yield a dark purple solid. The crude mixture of porphyrin isomers was purified by flash chromatography (silica, eluent: CHCl₃/MeOH, 19:1). Relevant fractions were combined, dried (Na₂SO₄) and evaporated in vacuo to yield 4 as a purple solid (526 mg, 3.9%); R_{f}=0.22 (silica, CHCl₃/MeOH, 19:1); mp>350° C. decomp.; ¹H N [270 MHz, CDCl₃] δ-2.79 (2H, br s, NH), 2.49 (3H, s, NHCOCH₃), 8.07 (2H, m, J*=8 Hz, 5-Ar-3,5-H), 8.21-8.28 (8H, m (overlapping), 5-Ar-2,6-H & 10, 15, 20-Py-2,6-H), 8.84-9.06 (8H, m, β-H), 9.10-9.15 (6H, m, 10, 15, 20-Py-3,5-H), 10.35 (1H, br s, NHCOCH₃); ¹³C NMR [67.5 MHz, CDCl₃] δ26.8, 106.9, 110.1, 110.2, 117.9, 121.1, 121.5, 122.1, 122.2, 123.3, 123.8, 123.9, 134.7, 140.1, 142.5, 145.1, 148.2, 149, 149.3, 149.4, 149.6, 150.1, 175.2; UV-vis (CH₂Cl₂) λₘₐₓ 418, 514, 548, 587, 644 nm; MS (MALDI-TOF) m/z 675 (M⁺, 100%).

(5) 5-(4-Aminophenyl)-10,15,20-tris(4-pyridyl)porphyrin

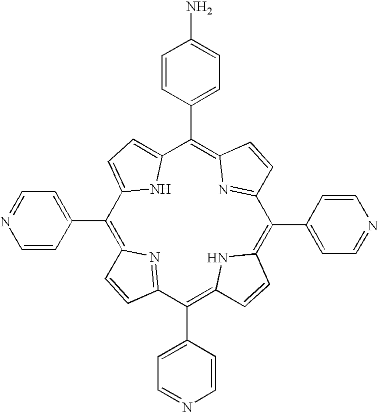

Porphyrin 4 (500 mg, 0.74 mmol) was dissolved in 18% HCl (100 mL) and the solution heated for 2 hours under reflux. Upon cooling the reaction mixture was evaporated in vacuo to yield a crude green solid. The solid was redissolved in a 9:1 mixture of dichloromethane/triethylamine (200 mL) and stirred for 10 min at room temperature. The solution was then washed with water (3×200 mL) and brine (200 mL), the organic layer separated and dried (Na₂SO₄). Excess solvent was evaporated in vacuo and the purple crude solid purified by flash chromatography (silica, eluent: CHCl₃/MeOH, 20:1). Relevant fractions were combined, dried (Na₂SO₄) and evaporated in vacuo to yield 5 as a purple solid (422 mg, 90.1%); R_{f}=0.31 (silica, CHCl₃/MeOH, 20:1); mp>350° C. decomp.; ¹H NMR [270 MHz, CDCl₃] δ-2.86 (2H, br s, NH), 4.09 (2H, br S, NH₂), 7.08 (2H, m, J*=8 Hz, 5-Ar-3,5-H), 7.98 (2H, m, J*=8 Hz, 5-Ar-2,6-H), 8.16 (6H, m, J*=5 Hz, 10, 15, 20-Py-2,6-H), 8.80-9.01 (8H, m, β-H), 9.04 (6H, m, J*=5 Hz, 10, 15, 20-Py-3,5-H); ¹³C NMR [67.5 MHz, CDCl₃] δ 113.6, 116.7, 117.4, 117.9, 122.7, 129.5, 131.7, 135.9, 146.5, 148.4, 148.5, 149.8, 150.2; UV-vis (CH₂Cl₂) λₘₐₓ 418, 515, 552, 592, 653 nm; MS (FAB) m/z 633 (M⁺, 100%).

(6) 5-(4-Acetamidophenyl)-10,15,20-tris(3-pyridyl)porphyrin

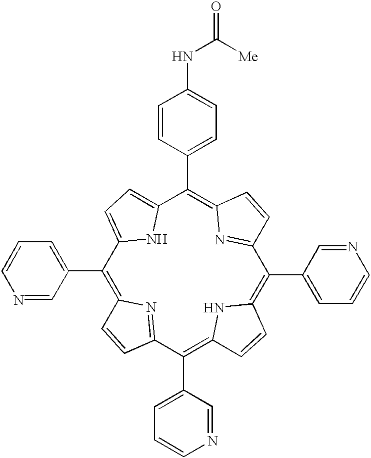

4-Acetamidobenzaldehyde (5 g, 0.031 mol) and 3-pyridinecarboxaldehyde (8.67 mL, 0.092 mol) were stirred in propionic acid (300 mL) at 90° C. Pyrrole (8.5 mL, 0.123 mol) was added and the mixture stirred under reflux for 30 min. Upon cooling the reaction mixture was evaporated in vacuo to yield a dark purple solid. The crude mixture of porphyrin isomers was purified by flash chromatography (silica, eluent: CHCl₃/MeOH, 19:1). Relevant fractions were combined, dried (Na₂SO₄) and evaporated in vacuo to yield 6 as a purple solid (0.96 g, 4.6%); R_{f}=0.26 (silica, CHCl₃/MeOH, 19:1); mp>350° C. decomp.; ¹H NMR [270 MHz, CDCl₃] δ-2.97 (2H, br s, NH), 2.17 (3H, s, NHCOCH₃), 7.40 (2H, m, J*=8 Hz, 5-Ar-3,5-H), 7.49 (3H, m, 10, 15, 20-Py-5-H), 7.98 (2H, m, J*=8 Hz, 5-Ar-2,6-H), 8.21-8.33(31H, m, 10, 15, 20-Py-6-H), 8.57-8.82 (11H, m (overlapping), 10, 15, 20-Py-4-H & β-H), 8.99 (1H, br s, NHCOCH₃), 9.26 (3H, m, 10, 15, 20-Py-2-H); UV-vis (CH₂Cl₂) λₘₐₓ 419, 516, 552, 592, 648 nm; MS (MALDI-TOF) m/z 675 (M⁺, 100%).

(7) 5-(4-Aminophenyl)-10,15,20-tris(3-pyridyl)porphyrin

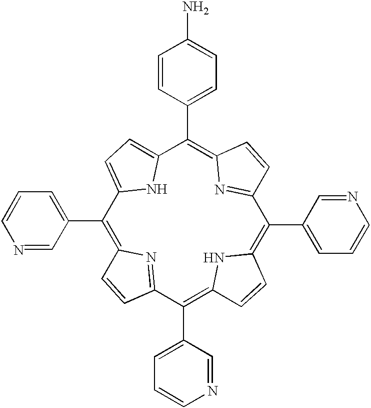

Porphyrin 6 (300 mg, 0.45 mmol) was dissolved in 18% HCl (100 mL) and the solution heated for 2 hours under reflux. Upon cooling the reaction mixture was evaporated in vacuo to yield a crude green solid. The solid was redissolved in a 9:1 mixture of dichloromethane/triethylamine (200 mL) and stirred for 10 min at room temperature. The solution was then washed with water (3×200 mL) and brine (200 mL), the organic layer separated and dried (Na₂SO₄). Excess solvent was evaporated in vacuo and the purple crude solid purified by flash chromatography (silica, eluent: CHCl₃/MeOH, 20:1). Relevant fractions were combined, dried (Na₂SO₄) and evaporated in vacuo to yield 7 as a purple solid (206 mg, 68.5%); R_{f}=0.38 (silica, CHCl₃/MeOH, 20:1); mp>350° C. decomp.; ¹H NMR [270 MHz, CDCl₃] δ-2.74 (2H, br s, NH), 3.93 (2H, br s, NH₂), 6.91 (2H, m, J*=8 Hz, 5-Ar-3,5-H), 7.67 (3H, m, 10, 15, 20-Py-5-H), 7.93 (2H, m, J*=8 Hz, 5-Ar-2,6-H), 8.48 (3H, m, 10, 15, 20-Py-6-H), 8.79-9.02 (11H, m (overlapping), 10, 15, 20-Py-4-H & β-H), 9.47 (3H, s, 10, 15, 20-Py-2-H); ¹³C NMR [67.5 MHz, CDCl₃] δ 113.3, 115.6, 116.1, 116.7, 121.9, 122.3, 131, 131.4, 131.8, 132.1, 132.3, 135.7, 137.5, 137.7, 137.8, 140.8, 146.3, 149, 149.2, 153.5, UV-vis (CH₂Cl₂) λₘₐₓ 420, 517, 553, 597, 649 nm; MS (MALDI-TOF) m/z 632 (M⁺, 100%).

(8) 5-(4-Acetamidophenyl)-15-(4-methoxyphenyl)porphyrin,

The DDP was synthesised according to the method of Dolphin et al.(11998 5-Phenyldipyrromethane and 5, 15-Diphenylporphyrin Org. Synth. 76, 287-293 incorporated herein by reference) The resulting mixture of three porphyrins was chromatographed, eluting initially with DCM to allow removal of 5,15-(4-methoxy)-DPP, and then continuing with ethyl acetate/DCM (1:4) to elute the required product as purple crystals (150 mg, 12%); R_{f}=0.40 (DCM/MeOH, 19:1); mp 305-307° C. (decomposed); UV-vis (DCM) λₘₐₓ (relative intensity) 410 (1.0), 502 (0.04), 538 (0.02), 578 (0.015), 630 (0.01) nm; ¹H NMR (270 M , CDCl₃) δ 10.35 (s, 2H, 10+20-H), 9.43 (d, 4H, J=4.8 Hz, β-H), 9.14 (d, 4H, J=4.8 Hz, β-H), 8.65 (m, 2H, J=7.2 Hz, 5-m-Ar), 8.22-8.12 (m, 4H, (overlapping), J=8.1 Hz, 5+15-o-Ar), 7.56 (m, 2H, J=8.1 Hz, 15-m-Ar), 4.14 (s, 3H, CH₃), −3.00 (br s, 2H, NH); MALDI-MS m/z 550.3 (M⁺, 100%).

(9) 5-(4-Aminophenyl)-15-(4-methoxyphenyl)porphyrin,

5-(4-Acetamido phenyl)-15-(4-methoxyphenyl)porphyrin 8 (1 eq., 100 mg, 0.182 mmol) was dissolved in 5 M aqueous HCl(100 mL) and the solution heated for 3 h under reflux. The hot reaction mixture was concentrated in vacuo to yield a crude green solid. The solid was re-dissolved in a mixture of DCM/triethylamine (9:1) (200 mL) and stirred for 10 min at room temperature. The solution was then washed with water (3×200 mL), saturated brine (200 mL) and the organic layer separated and dried (Na₂SO₄), then concentrated in vacuo. The crude purple solid was chromatographed, eluting with DCM, and gave the desired porphyrin as a purple crystalline solid (51 mg, 54%), R=0.30 (DCM), mp 300° C. (decomposed); UV-vis (DCM) λₘₐₓ (relative intensity) 410 (1.0), 503 (0.045), 538(0.02), 578 (0.015), 630 (0.005) nm; Fluorescence (DCM) λₘₐₓ 634 nm (λ excitation=410 nm); ¹H NMR (270 Mz, CDCl₃) δ 10.30 (s, 2H, 10+20-H), 9.39 (d, 4H, J=4.9 Hz, β-B), 9.17 (d, 2H, J=4.9 Hz, β-H), 9.10 (d, 2H, J=4.9 Hz, β-H), 8.19 (m, 2H, J=8.8 Hz, 15-o-Ar), 8.07 (m, 2H, J=8.1 Hz, 5-o-Ar), 7.35 (m, 2H, J=8.8 Hz, 15-m-Ar), 7.14 (m, 2H, J=8.1 Hz, 5-m-Ar), 4.13 (s, 3H, CH₃), 4.08 (br s, 2H, NH), −3.06 (br s, 2H NH); MALDI-MS m/z 508.3 ([M+1]⁺, 100%). ES-HRMS calcd. for C₃₃H₂₆N₅O ([M+1]⁺) 508.2137, found 508.2144.

(10) 17,18-Dihydroxy-5-(4-aminophenyl)-15-(4-methoxyphenyl)chlorin and

(11) 7,8-dihydroxy 5-(4-aminophenyl)-15-(4-methoxyphenyl)chlorin Regioisomers,

Porphyrin 9 (28 mg, 55.2 μmol) was converted into the required mixture of chlorin regioisomers following the procedure of Sutton et al.(2000 Functionalised diphenylchlorins and bacteriochlorins—their synthesis and bioconjugation for targeted photodynamic therapy and tumour cell imaging J. Porphyrin and Phthalocyanines 4, 655-658) The crude reaction mixture was then chromatographed, eluting with 1% MeOH in DCM. First, some un-reacted starting material was eluted, then the higher R_{f} chlorin isomer 10 as a brown-purple crystalline solid; R_{f}=0.28 (DCM/MeOH, 19:1). The lower R_{f} isomer 11 was obtained by further elution with 2.5% MeOH in DCM and gave also a brown-purple crystalline solid (R_{f}=0.17 (DCM/MeOH, 19:1).

High R_{f} chlorin regioisomer (17,18-dihydroxy-15-(4-methoxy phenyl)-5-(4-aminophenyl)chlorin assigned previously(26)) (7.0 mg, 24%), mp 165-167° C. (decomposed); UV-vis (DCM) λₘₐₓ (relative intensity) 401 (0.99), 414 (1.0), 503 (0.08), 535(0.07), 582 (0.035), 636 (0.22) nm; Fluorescence (DCM) λₘₐₓ 639 nm (λ excitation=412 nm); ¹H NMR (270 MHz, 10% CD₃OD in CDCl₃) δ 9.95 (s, 1H, 10-H), 9.42 (s, 1H, 20-H), 9.17 (d, 1H, J=4.8 Hz, β-H), 9.03 (d, 1K J=4.0 Hz, β-H), 8.97 (s, 2H, β-H) 8.78 (d, 1H, J=4.8 Hz, β-H), 8.51 (d, 1H, J=4.8 Hz, β-H), 8.05 (m, 2H, J=8.9 Hz, o-Ar), 794 (m, 2H, J=8.1 Hz, o′-Ar), 7.25 (m, 2H, J=8.9 Hz, m-Ar), 7.12 (m, 2H, J=8.1 Hz, m′-Ar), 642 (d, 1H, J=6.5 Hz, 17-H), 6.03 (d, 1H, J=6.5 Hz, 18-H), 4.08 (s, 3H., CH₃), (NH's exchanged & OH's not observed); MALDI-MS m/z 542.2 ([M+H]⁺, 100%); ES-HRMS calcd. for C₃₃H₂₈N₅O₃ ([M+H]⁺) 542.2192, found 542.2187.

Low R_{f} chlorin regioisomer (7,8-dihydroxy-5-(4-aminophenyl)-15-(4-methoxyphenyl)chlorin) (8.5 mg, 30%), mp 168-171° C. (decomposed); UV-vis (DCM) λₘₐₓ (relative intensity) 401(0.99), 413 (1.0), 507 (0.08), 536 (0.06), 586 (0.025), 637 (0.20) nm; Fluorescence (DCM) λₘₐₓ 639 nm (λ excitation=412 nm); ¹H NMR (270 MHz, 10% CD₃OD in CDCl₃) δ 9.96 (s, 1H, 20-H), 9.40 (s, 1H, 10-H), 9.18 (d, 1H, J=4.8 Hz, β-H), 9.05 (d, 1H, J=4.8 Hz, 8-H), 8.98 (d, 1H, J=4.0 Hz, β-H) 8.92 (d, 1H, J=4.0 Hz, β-H), 8.74 (d, 1H, J=4.0 Hz, β-H), 8.58 (d, 1H, J=4.0 Hz, β-H), 8.13 (m,1H, J=8.9 Hz, o-Ar), 8.08 (m, 1H, J=8.9 Hz, o-Ar), 7.95 (m, 1H, J:=8.1 Hz, o′-Ar), 7.79 (m, 1H, J=8.1 Hz, o′-Ar), 7.36 (m, 1H, J=8.9 Hz, m-Ar), 7.30 (m, 1H, J=8.9 Hz, m-Ar), 7.11 (m, 1H, J=8.1 Hz, m′-Ar), 7.05 (m, 1H, J=8.1 Hz, m′-Ar), 6.42 (d, 1H, J=6.5 Hz, 7-H), 6.09 (d, 1H, J=6.5 Hz, 8-H), 4.11 (s, 3H, CH₃), (NH's exchanged & OH's not observed); MALDI-MS m/z 542.2 ([M+H]⁺, 100%) ES-HRMS calcd. for C₃₃H₂₈N₅O₃ ([M+H]⁺) 542.2192, found 542.2185.

(12) 5-(4-Fluorenylmethylaminophenyl)-15-(4-methoxyphenyl)porphyrin,

To a stirred solution of porphyrin 9 (28 mg, 55 mol) in anhydrous 1,4-dioxane (2.5 mL) was added solid sodium hydrogen carbonate (6 eq. 28 mg, 0.33 mmol). To this mixture was then added a solution of 9-fluorenylmethyl chloroformate (2 eq., 0.11 mmol, 28.5 mg) in 1,4-dioxane (0.5 mL) under N₂. The reaction flask was covered with aluminium foil to exclude light and stirred at room temperature for a period of 3 h. At this time the reaction was complete (as monitored by TLC). The 1,4-dioxane was removed in-vacuo and the residue partitioned between water (25 mL) and DCM (2×25 mL). The combined organic extracts were washed with saturated brine (25 mL) then dried (Na₂SO₄), filtered and concentrated in vacuo. The required porphyrin was obtained by chromatography, eluting with DCM. The desired porphyrin was obtained as purple crystals (38 mg, 95%), R_{f} 0.39 (DCM), mp 292-295° C. (decomposed); UV-vis (DCM) λₘₐₓ (relative intensity) 410 (1.0), 505 (0.042), 541 (0.02), 578 (0.015), 633 (0.01) nm; Fluorescence (DCM) λₘₐₓ 635 nm (λ excitation=410 nm); ¹H NMR (270 MHz, CDCl₃) δ 10.35 (s, 2H, 10+20-H), 9.69 (br. s, 1H, NH), 9.44 (d, 4H, J=4.8 Hz, β-H), 9.12 (d, 4H, J=4.8 Hz, β-H), 8.20-8.17 (in (overlapping), 4H, J=8.1 Hz, 5+15-o-Ar), 7.85 (m, 4H, 5+15-m-Ar), 7.76-7.66 (m, 2H, fluoreno-Ar), 7.51-7.30 (m, 6H, fluoreno-Ar), 4.69 (d, 2H, J=7.2 Hz, CH₂), 4.30 (t, 1H, J=7.2 Hz, CH), 4.13 (s, 3H, CH₃), −3.15 (br. s, 2H, NH), MALD-MS m/z 731.5 ([M+H]⁺, 100%), 508.3 ([M-FMOC+2]+, 50%); ES-HRMS calcd. for C₄₈H₃₆N₅O₃ ([M+H]⁺) 730.2818, found 730.2809.

(13, 14) cis/trans-7,8,17,18-Tetrahydroxy-5-(4-fluorenylmethylaminophenyl) 15-(4-methoxyphenyl) Bacteriochlorins

Porphyrin 12 (35 mg, 48.0 μμmmol) was converted into the required mixture of bacteriochlorin stereoisomers by minor modification of the procedure of Sutton et al.(2000 Functionalised diphenylchlorins and bacteriochlorins—their synthesis and bioconjugation for targeted photodynamic therapy and tumour cell imaging J. Porphyrin and Phthalocyanines 4, 655-658-incorporated herein by reference) (reaction carried out using 1,4-dioxane (5 mL) to allow dissolution of 12). The crude reaction mixture was chromatographed, eluting initially with 1% MeOH in DCM to remove chlorin by-products. Further elution with 2% MeOH/DCM allowed isolation of both stereoisomeric bacteriochlorin tetrols. The higher R_{f}-trans bacteriochlorin isomer 13 was isolated as a pink-green crystalline solid, (6 mg, 15%), R_{f}=0.25 (DCM/MeOH, 19:1), mp 142-145° C. (decomposed); UV-vis (DCM) λₘₐₓ (relative intensity) 374 (1.0) 512 (0.23), 702 (0.52) nm; Fluorescence (DCM) λₘₐₓ 708 nm (λ excitation=512 nm); ¹H NMR (270 MHz, 10% CD₃OD in CDCl₃) δ 9.20 (s, 2H, 10+20-H), 8.78 (d, 2H, J=4.0 Hz, β-H), 8.36 (d, 2H, J=4.0 Hz, β-H), 7.95 (m, 2H, o-Air), 7.85 (m, 2H, J=7.3 Hz, fluoreno-Ar), 7.79 (m, 2H o′-Ar), 7.65 (m, 2H, m-Ar), 7.47-7.38 (m, 6H, fluoreno-Ar), 7.24 (m, 2H, m-Ar), 6.27-6.24 (m, 2H, 7+17-H), 5.85 (m, 2H, 8+18-H), 4.65 (d, 2H, J=7.2 Hz, CH₂), 4.39 (t, 1H, J=7.2 Hz, CH), 4.06 (s, 31H, CH₃), −1.94 (br s (partly exchanged), 2H1, NH), (OH's not observed); MALDI-MS m/z 800.4 ([M+H]⁺, 100%); ES-HRMS calcd. for C₄₈H₄₀N₅O₇ ([M+H]⁺) 798.2927, found 798.2921.

The lower R_{f} cis-bacteriochlorin isomer 14 was isolated as a pink-green crystalline solid, (8.5 mg, 21%), R_{f}=0.2 (DCM/MeOH, 19:1), mp 148-150° C. (decomposed); UV-vis (DCM) λₘₐₓ (relative intensity) 374 (1.0) 512 (0.24), 703 (0.54) nm; Fluorescence (DCM) λₘₐₓ 708 nm (λ excitation=512,nm); ¹HNMR (270 MHz, 10% CD₃OD in CDCl₃) δ 9.12 (s, 2H, 10+20-H), 8.76 (d, 2H, J=4.8 Hz, β-H), 8.34 (d (overlapping), 2H, J=4.8 Hz, β-H), 8.02 (m, 2H, o-Ar), 7.85 (m (obscured), 2H, J=8.0 Hz, o′-Ar), 7.83 (m, 2H, J=7.3 Hz, fluoreno-Ar), 7.76 (m, 2H, J=8.0 Hz, m-Ar), 7.50-7.38 (m, 6H, fluoreno-Ar), 7.24 (m, 2H, m-Ar), 6.27-6.23 (m, 2H, 7+17-H), 5.85-5.82 (m, 2H, 8+18-H), 4.65 (d, 2H, J=7.2 Hz, CH₂), 4.39 (t, 111, J=7.2 Hz, CH), 4.05 (s, 311, CH₃), −1.88 (br. s (partly exchanged), 2H, NM, (OH's not observed); MALDI-MS m/z 800.4 ([M+H]⁺, 100%); ES-HRMS calcd. for C₄₈H₄₀N₅O₇ ([M+H]⁺) 798.2927, found 798.2921.

(15) 5-(3,4,5-Trismethoxyphenyl)dipyrromethane

3,4,5-Trismethoxybenzaldehyde (5.0 g, 25.5 mmol) was dissolved in freshly distilled pyrrole (75 ml) and the solution degassed by bubbling with dry N₂ for 10 min. TFA (0.075 eq., 0.15 ml, 1.91 mmol) was added and the mixture stirred under N₂ until no starting aldehyde could be detected by TLC (ca. 10 min). The reaction mixture was concentrated in vacuo at water aspirator pressure (evaporator water bath temp 75° C.) then under high vacuum for 16 h to remove excess pyrrole. The crude product was recrystallised from hot ethylacetate/nHexane and afforded the required dipyrromethane as a white solid, (5.41 g, 68%). νₘₐₓ (nujol mull)/cm⁻¹ 3378 (br. NH), 1594 (C═C), 1233, 1040; UV-VIS (MeOH) λₘₐₓ/(rel. intensity) 222 (1.0), 280 (0.75) nm; δH(270 MHz; CDCl₃) 8.07 (2H, br. s, NH), 7.53 (2H, m, 1-H), 6.68 (2H, s, 2′-H), 6.37 (2H, m, 2-H), 5.93 (2H m, 3-H), 5.38 (1H, s, methane), 3.80 (3H, s, 4′-OCH₃), 3.73 (6H, s, 3′+5′-OCH₃); δC(68 MHz; CDCl₃) 152.7 (CH, 2′-C), 137.3 (q, 3′+5′-C), 136.1 (q, 4′-C), 131.8 (q, 4-C), 116.7 (CH, 1-C), 107.9 (CH, 2-C), 106.6 (CH, 3-C), 104.9 (q, 1′-C), 60.3 (CH₃), 55.5 (CH₃), 43.7 (CH, methane); MS (MALDI) m/e 311.2 (100%, (M−1)⁺).

(16) 5-(4-Acetomidophenyl)dipyrromethane

The dipyrromethane was synthesised using the general procedure detailed above using the same molar quantity of starting aldehyde. The crude reaction mixture was chromatographed on flash silica-gel (350 ml), (dry loaded on to 50 ml flash silica-gel from ethylacetate) and eluted with 40% ethylacetate/DCM and afforded the pure product as an off white solid, (4.3 g, 50%): νₘₐₓ (nujol mull)/cm⁻¹ 3409 (NH, amide), 3248 (br. NH), 1650 (C═O), 1593 (C—C), 1320, 1009; UV-VIS (MeOH) λₘₐₓ/(rel. intensity) 224 (1.0) nm; δH(270 MHz; CDCl₃) 8.00 (2H, br. s, NH), 7.40 (2H d, J=8.5 Hz, o-Ar), 7.30 (1H, br. s, NH-acetomido), 7.13 (2H, d, J=8.5 Hz, m-Ar), 6.68 (2H, m, 1-H), 6.16 (2×m, 2-H), 5.90 (2H, m, 3-H), 5.42 (1H, s, methane), 2.14 (3H, s, NHCH₃); δC(68 MHz; CDCl₃) 168.4 (q, COCH₃), 138.2 (q, 4′-C), 136.5 (q, 2′-C), 132.4 (q, 4-C), 128.9 (CH, 2′-C), 120.3 (CH, 3′-C), 117.2 (CH, 1-C), 108.4 (CH, 2-C), 107.1 (CH, 3-C), 43.4 (CH, methane), 24.5 (CH₃); MS (MALDI) m/e 279.4 (100%, (M)⁺).

(17) 5-(4-Methoxyphenyl)dipyrromethane

The dipyrromethane was synthesised using the general procedure detailed above using the same molar quantity of starting aldehyde. The crude reaction mixture was chromatographed on flash silica-gel (350 ml), (dry loaded on to 50 ml flash silica-gel from ethylacetate) and eluted with 30% nHexane/DCM and afforded the pure product as an off white solid, (4.3 g, 50%): νₘₐₓ (nujol mull)/cm⁻¹, 3382 (br. NH), 1598 (C═C), 1300, 1050; UV-VIS (MeOH) λₘₐₓ/(rel. intensity) 224 (1.0) nm; δH(270 MHz; CDCl₃) 7.87 (2H, br. s, NM, 7.10 (2H, d, J=8.8 Hz, m-Ar), 6.83 (2H, d, J=8.8 Hz, o-Ar), 6.66 (2H, m, 1-H), 6.14 (2H, m, 2-H), 5.89 (2H, m, 3-H), 5.40 (1H s, methane); MS (MALDI) m/e 252.4 (100%, (M)⁺).

### EXAMPLE 1

5-(4-Isothiocyanatophenyl)-10,15,20-tris(3,5-dihydroxyphenyl)porphyrin

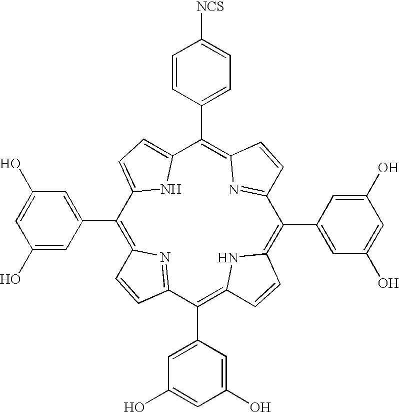

To a stirred solution of 3 (100 mg, 0.137 mmol) in freshly distilled THF (25 mL) was added 1,1′-thiocarbonyldi-2(1H)-pyridone (64 mg, 0.276 mmol). The reaction was allowed to proceed under argon for 4 hours at room temperature. Excess solvent was evaporated in vacuo to yield a crude purple solid. The solid was dissolved in a minimal amount of chloroform/methanol (9:1) and purified by flash chromatography (silica, eluent: CHCl₃/MeOH, 9:1). Relevant fractions were combined, dried (Na₂SO₄) and evaporated in vacuo to yield the above compound as a purple solid (67.5 mg, 63.8%); R_{f}=0.29 (silica, CHCl₃/MeOH, 9:1); mp>350° C. decomp.; ¹H NMR [270 MHz, CDCl₃/CD₃OD, 3:1] δ 6.77 (3H, s, 10, 15, 20-Ar-4-H), 7.12 (6H, s, 10, 15, 20-Ar-2,6-H), 7.64 (2H, m, J*=8 Hz, 5-Ar-3,5-H), 8.19 (2H, m, J*=8 Hz, 5-Ar-2,6-H), 8.76-9.0 (8H, m, β-H); ¹³C NMR [67.5 MHz, CDCl₃/CD₃OD, 3:1] 101.9, 107.1, 114.7, 117.6, 119.9, 120, 120.1, 123.9, 130.9, 134, 135.2, 136.1, 141.2, 142, 143.6, 155.8; UV-vis(MeOH) λₘₐₓ 422, 516, 552, 592, 648 nm; HRMS (ES) m/l calc'd for C₄₅H₂₉N₅O₆S [M+H]⁺ 768.1914, found 768.1908.

### EXAMPLE 2

5-(4-Isothiocyanatophenyl)-10,15,20-tris(4-pyridyl)porphyrin

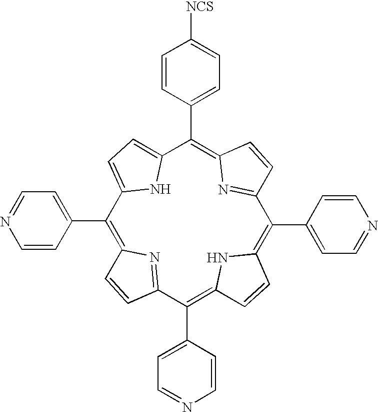

To a stirred solution of 5 (100 mg, 0.158 mmol) in freshly distilled dichloromethane (20 mL) was added 1,1′-thiocarbonyldi-2(1H)-pyridone (320 mg, 1.38 mmol). The reaction was allowed to proceed under argon for 4 hours at room temperature. Excess solvent was evaporated in vacuo to yield a crude purple solid. The solid was dissolved in a minimal amount of chloroform and purified by flash chromatography (silica, eluent: CHCl₃MeOH, 49:1). Relevant fractions were combined, dried (Na₂SO₄) and evaporated in vacuo to yield the above compound as a purple solid (104 mg, 97.5%); R_{f}=0.57 (silica, CHCl₃/MeOH, 49:1); mp>350° C. decomp.; ¹H NMR [270 MHz, CDCl₃] δ-2.91 (2H, br s, NH), 7.65 (2H, m, J*=8 Hz, 5-Ar-3,5-H), 8.15-8.21 (8H, m (overlapping), 10, 15, 20-Py-2,6-H & 5-Ar-2,6-H), 8.67 (8H, br s, β-H), 9.06 (6H, m, J*=5 Hz, 10, 15, 20-Py-3,5-H); ¹³C NMR [67.5 M, CDCl₃]δ 117.4, 117.6, 119.7, 124.7, 129.3, 131.6, 135.4, 136.9, 140.6, 148.4, 149.8; UV-vis (CH₂Cl₂) λₘₐₓ 417, 514, 548, 587, 643 nm; HRMS (ES) m/z calc'd for C₄₂H₂₆N₈S (M+H) 675.2079, found 675.2078.

### EXAMPLE 3

5-(4-Isothiocyanatophenyl)-10,15,20-tris(3-pyridyl)porphyrin (160)

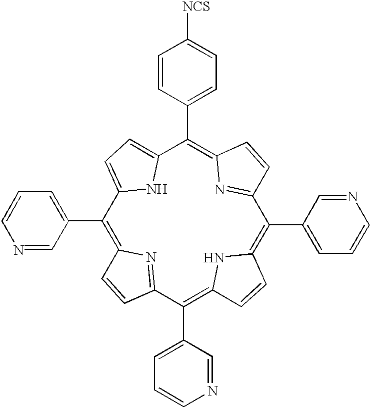

To a stirred solution of 7 (200 mg, 0.316 mmol) in freshly distilled dichloromethane (40 mL) was added 1,1′-thiocarbonyldi-2(1H)-pyridone (640 mg, 2.76 mmol). The reaction was allowed to proceed under argon for 17 hours at room temperature. Excess solvent was evaporated in vacuo to yield a crude purple solid. The solid was dissolved in a minimal amount of chloroform and purified by flash chromatography (silica, eluent: CHCl₃/MeOH, 49:1). Relevant fractions were combined, dried (Na₂SO₄) and evaporated in vacuo to yield the above compound as a purple solid (171 mg, 80.3%); R_{f}=0.55 (silica, CHCl₃/MeOH, 49:1); mp>350° C. decomp:; ¹H NMR [270 MHz, CDCl₃] δ-2.83 (29H br s, NW, 7.65 (2H, m, J*=8 Hz, 5-Ar-3,5-H), 7.78 (3H, m, 10, 15, 20-Py-5-H), 8.20 (2H, m, J*=8 Hz, 5-Ar-2,6-H), 8.54 (3H, m, 10, 15, 20-Py-6-H), 8.83-8.88 (8H, m, β-H), 9.07 (3H, m, 10, 15, 20-Py-4-H), 9.07 (3H, s, 10, 15, 20-Py-2-H); ¹³C NMR [67.5 MHz, CDCl₃] δ 116.6, 122.1, 124.2, 131.5, 135.5, 137.7, 140.8, 140.9, 149.2, 153.5; UV-vis (CH₂Cl₂) λₘₐₓ 421, 513, 547, 587, 657 nm, MS (MALDI-TOF) m/z 674 (M⁺, 100%).

### EXAMPLE 4

5-(4-Isothiocyanatophenyl)-10,15,20-tris(4-N-methylpyridiniumyl) porphyrin triiodide

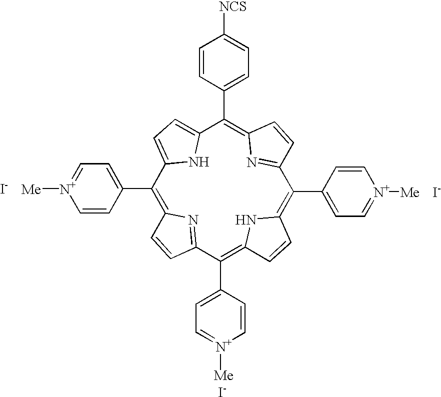

To a solution of Example 2 (50 mg, 0.074 mmol) in anhydrous DMF (10 mL, distilled from CaH₂, 0.1 torr) was added iodomethane (1 mL, 0.016 mol). The reaction was stirred under argon for 3 hours at room temperature, monitored by TLC (normal phase silica) in a water/saturated aqueous potassium nitrate/acetonitrile (1:1:8) solvent system. Upon reaction completion excess DMF was evaporated in vacuo (0.1 torr) at 30-40° C. to yield the above compound as a lustrous purple solid (77 mg, 95%); R_{f}=0.32 (silica, H₂O/sat.aq. KNO₃/MeCN, 1:1:8); mp>350° C. decomp.; ¹H NMR[270 MHz, (CD₃)₂SO] δ-3.03 (2H, br s, NH), 4.74 (9H, br s, N—CH₃-pyridine), 7.96 (2H, n, J*=8 Hz, 5-Ar-3,5-H), 8.32 (2H, m, J*=8 Hz, 5-Ar-2,6-H), 9.03 (6H, m, J*=6 Hz, 10, 15, 20-Py-2,6-H), 9.16 (8HS m, β-H, 9.50 (6H, m, J*=6 Hz, 10, 15, 20-Py-3,5-H); ¹³C NMR [67.5 MHz, (CD₃)₂SO] δ 47.9, 114.7, 115.3, 121.1, 124.8, 130.6, 132, 134.7, 135.4, 139.7, 144.1, 156.3, 156.4; UV-vis (H₂O) λₘₐₓ 423, 520, 585 nm; MS (FAB) m/z 719 (M⁺, 100%), 704 (M-CH₃, 26%), 689 (M-2CH₃, 20%), 674 (M-3CH₃, 5%); HRMS (ES) m/z calc'd for C₄₅H₃₅N₈S (M+H) 719.2705, found 719.2686.

### EXAMPLE 5

5-(4-Isothiocyanatophenyl)-10,15,20-tris(3-N-methylpyridiniumyl) porphyrin triiodide

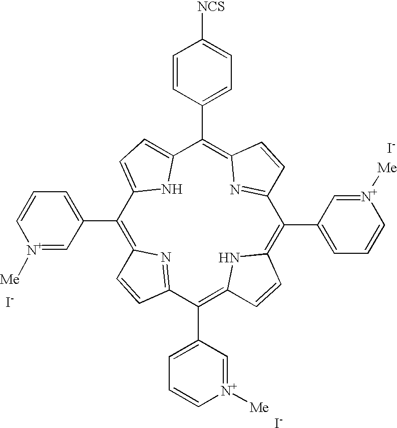

To a solution of Example 3 (50 mg, 0.074 mmol) in anhydrous DMF (5 mL, distilled from CaH, 0.1 torr) was added iodomethane (1 mL, 0.016 mol). The reaction was stirred under argon for 4 hours at room temperature, monitored by TLC (normal phase silica) in a water/saturated aqueous potassium nitrate/acetonitrile (1:1:8) solvent system. Upon reaction completion excess DMF was evaporated iii vacuo (0.1 torr) at 30-40° C. to yield the above compound as a lustrous purple solid (72 mg, 89%); R_{f}=0.46 (silica, H₂O/sat.aq. KNO₃/MeCN, 1:1:8); mp>350° C. decomp.; ¹H NMR [270 MHz, (CD₃)₂SO]δ-3.07 (2H, br s, NH), 4.69 (9H, br s, N—CH₃-pyridine), 7.97 (2H, m, J*=8 Hz, 5-Ar-3,5-H), δ 31 (2H, m, J*=8 Hz, 5-Ar-2,6-H), 8.64 (3H, m, 10, 15, 20-Py-5-H), 9.03-9.25 (8H, m, β-H), 9.35 (3H, m, 10, 15, 20-Py-6-H), 9.57 (3H, m, 10, 15, 20-Py-4-H), 10.03 (3H, s, 10, 15, 20-Py-2-H); ¹³C NMR [67.5 MHz, (CD₃)₂SO] δ 48.3, 112.3, 112.9, 120.7, 124.8, 126.3, 126.4, 126.6, 130.6, 132.1, 132.3, 132.4, 132.6, 132.8, 133.1, 133.4, 134.7, 135.4, 139.8, 139.9, 140, 145.5, 145.6, 147.4, 147.5, 147.8, 147.9, 148.5, 155.9; UV-vis (H120) λₘₐₓ 419, 516, 552, 581′, 637 nm; MS (MALDI-TOF) m/z 689 ([M-2CH₃]⁺, 100%).

### EXAMPLE 6

5-(4-Isothiocyanatophenyl)-10,15,20-tris(4-N-methylpyridiniumyl) porphyrin Trichloride

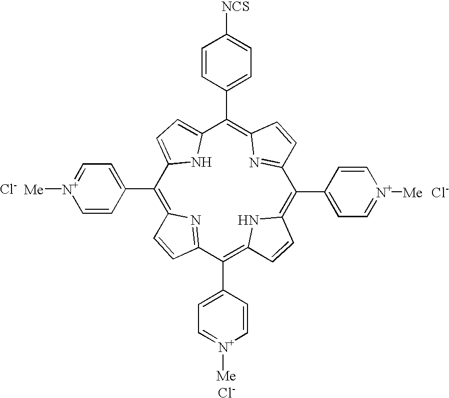

To a solution of Example 4 (30 mg, 0.027 mmol) in anhydrous methanol (30 mL) was added Amberlite® IRA 400 (1 g) and the mixture stirred for 1 hour at room temperature. Amberlite® IRA 400 resin was filtered under vacuum and the porphyrin filtrate recovered, dried (Na₂SO₄) and evaporated in vacuo to yield the above compound as a water soluble purple solid (22 mg, 96.4%). Porphyrins of Examples 4 and 6 were distinguished only by their respective solubility in water.

### EXAMPLE 7

5-(4-Isothiocyanatophenyl)-10,15,20-tris(4-N-methylpyridiniumyl) porphyrin Trichloride

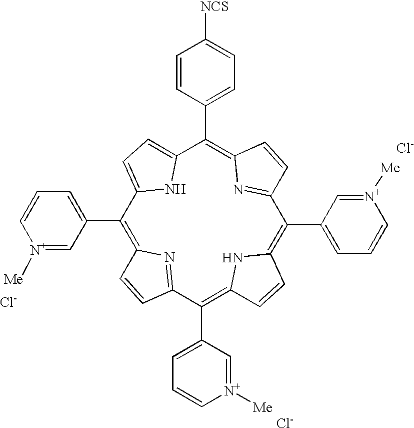

To a solution of 5 (30 mg, 0.027 mmol) in anhydrous methanol (30 mL) was added Amberlite® IRA 400 (1 g) and the mixture stirred for 1 hour at room temperature. Amberlite® IRA 400 resin was filtered under vacuum and the porphyrin filtrate recovered, dried (Na₂SO₄) and evaporated ill vacuo to yield the above compound as a water soluble purple solid (21 mg, 92.0%). Porphyrins of Examples 5 and 7 were distinguished only by their respective solubility in water.

### EXAMPLE 8

17,18-Dihydroxy-5-(4-isothiocyanatophenyl)-15-(4-methoxyphenyl)chlorin, The higher R_{f} regioisomeric chlorin 10 (17.5 mg, 23.2 Kmol) was converted into the corresponding isothiocyanate according to the following method. To a stirred solution of 10 (1 eq., 50 mg, 0.099 mmol) in freshly distilled DCM (20 mL) was added 1,1′-thiocarbonyldi-2(1H)-pyridone (2 eq., 46 mg, 0.198 mmol). The reaction was allowed to stir under argon for 2 h at room temperature, after which the reaction mixture was filtered, and concentrated then chromatographed, eluting with 1% MeOH in DCM to afford the title compound. The title compound was isolated as a brown-purple crystalline solid, (17 mg, 90%), R_{f}=0.36 (DCM/MeOH, 19:1), mp 155-158° C. (decomposed); UV-vis (DCM) λₘₐₓ (relative intensity) 410 (1.0) 505 (0.09), 534 (0.06), 586 (0.04), 637 (0.18) nm; Fluorescence (DCM) λₘₐₓ 639 nm (λ excitation=412 nm); ¹H NMR (270 MHz, 10% CD₃OD in CDCl₃) δ 10.0 (s, 1H, 10-H), 9.45 (s, 1H, 20-H), 9.20 (d, 1H, J=4.8 Hz, β-H), 9.06 (d, 1H, J=4.0 Hz, β-H), 9.02 (d, 1H, J=4.8 Hz, β-H) 8.84 (d, 1H, J=4.8 Hz, β-H), 8.64 (d, 1H, J=4.0 Hz, β-H), 8.55 (d, 1H, J=4.8 Hz, β-H, 8.21 (m, 1H, J=8.1 Hz, 5-o-4r), 8.15 (m, 1H, J=8.1 Hz,-o-Ar), 8.05 (m, 1H, J=8.9 Hz, 15-o-Ar) 7.93 (m, 1H, J=8.9 Hz, 15-o-Ar), 7.65 (m, 2H, 5-m-Ar), 7.24 (m, 2H, 15-m-Ar), 6.43 (d, 1H, J=6.5 Hz, 17-H), 6.04 (d, 1H, J=6.5 Hz, 18-H), 4.08 (s, 3H, CH₃), (NH's exchanged & OH's not observed); MALDI-MS m/z 583.7 ([M=H]⁺, 100%); ES-HRMS calcd. for C₃₄H₂₆N₅O₃S ([M+H]⁺) 584.1757, found 584.1756.

### EXAMPLE 9

cis-7,8,17,18-Tetrahydroxy-5-(4-isothiocyanatophenyl)-15-(4-methoxyphenyl)bacteriochlorin

The cis-bacteriochlorin 14 (8.5 mg, 10.7 μmol) in 25% MeOH in DCM (1.25 mL) was treated with piperidine (50 eq., 53 μl, 0.53 mmol) and left to stir for a period of 3 h at room temperature under N₂ with light excluded. The reaction mixture was concentrated in vacuo (0.1 torr) to remove all traces of piperidine. The crude amine was then converted into the required isothiocyanate following the procedure described above The cis-bacteriochlorin_isothiocyanate was isolated as a pink-green crystalline solid (5.0 mg, 76%), R_{f}=0.40 (DCM/MeOH, 19:1), mp 132-135° C. (decomposed); UV-vis (DCM) λₘₐₓ (relative intensity) 375 (1.0) 516 (0.22), 702 (0.48) nm; Fluorescence λₘₐₓ 709 nm (λ excitation=516 nm); ¹H NMR (270 MHz, 10% CD₃OD in CDCl₃) δ 9.20 (s, 1H, meso-H), 9.18 (s, 1H, meso-H), 8.77 (d, 2H, J=4.8 Hz, β-H), 8.40 (d, 1H, J=4.8 Hz, β-H), 8.34 (d, 1×J=4.8 Hz, β-H), 8.14 (m, 2H, o-Ar), 8.05 (m, 2H, o′-Ar), 7.42-7.08 (m, 4H, 5+15-m-Ar-), 6.20 (m, 2H, 7+17-H), 5.98 (m, 1H, 8-H), 5.93 (m, 1H, 18-H), 4.04 (s, 3H, CH₃), −1.80 (br s, 2H, partly exchanged-NH), (OH's not observed); MALDI-MS m/z 618.9 ([M+H]⁺, 100%); ES-HRMS calcd. for C₃₄H₂₈N₅O₅S ([M+H]⁺) 618.1815, found 618.1810.

### EXAMPLES 10, 11, 12, 13

17,18-Dihydroxy-5-(4-acetamidophenyl)-15-(4-methoxyphenyl)chlorin/7,8-dihydroxy-5-(4-acetamidophenyl)-15-(4-methoxyphenyl)chlorin regioisomers, and cis/trails-7,8,17,18-tetrahydroxy-5-(4-acetamidophenyl)-15-(4-methoxyphenyl)bacteriochlorin stereoisomers,

Porphyrin 8 (100 mg, 0.18 mmol) was converted, in a single reaction, to a mixture of chlorin diols/bacteriochlorin tetrols following the procedure of Sutton et al. After 38 h the reaction was stopped. The crude reaction mixture was then chromatographed, eluting with 2% MeOH in DCM to give first, some un-reacted starting material then the higher R_{f} chlorin isomer of Example 10 as a brown-purple crystalline solid (5 mg, 5%). The lower R_{f} isomer of Example 11 was obtained by further elution with 3.5% MeOH in DCM and gave also a brown-purple crystalline solid (7.0 mg, 7%). Further elution with 5% MeOH in DCM afforded the required trans/cis-bacteriochlorin tetrols of Examples 12 and 13 respectively as pink/green solids (5.0 mg, 5%) and (7.0 mg, 7%) respectively. High R_{f} chlorin regioisomer of Example 10 (17,18-dihydroxy-15-(4-methoxyphenyl)-5-(4-acetamidophenyl) chlorin assigned on the basis of past data)(26) R_{f}=0.40 (DCM/MeOH, 37:3), mp 186-188° C. (decomposed); UV-vis (DCM) λₘₐₓ (relative intensity) 410(1.0), 505.5 (0.12), 535 (0.08), 585.5 (0.05), 637 (0.18) nm; Fluorescence (DCM) λₘₐₓ 639 nm (λ excitation=410 nm); ¹HNMR (270 MHz, CDCl₃) δ 9.97 (s, 1H, 10-H), 9.42 (s, 1H, 20-H), 9.19 (d, 1H J=4.0 Hz, 6-H), 9.03 (d, 1H, J=4.0 Hz, β-H), 8.98 (d, H, J=4.8 Hz, β-H) 8.89 (d, 1H, J=4.0 Hz, β-H), 8.70 (d, 11, J=4.8 Hz, β-H), 8.52 (d, 1H, J=4.8 Hz, β-H), 8.14-8.10 (m, 3H, 5-o/m-Ar), 7.96-7.82 (m, 3H, 5+15-o′m-Ar), 7.50 (s, 1H, NH), 7.34 (m, 2H, 15-m-Ar), 6.48 (m, 1H, 17-H), 6.20 (m, 1H, 18-H), 4.08 (s, 3H, CH₃), 2.38 (s, 3H, CH₃), −1.89, −2.20 (s, 2H, NH); MALDI-MS m/z 582.6 ([M+H]⁺, 100%); ES-HRMS calcd. for C₃₅H₂₈N₅O₄ ([M+H]⁺) 582.2141, found 582.2137. Low R_{f} chlorin regioisomer of Example 11 (7,8-dihydroxy-5-(4-aminophenyl)-15-(4-methoxyphenyl)chlorin) R_{f}=0.35 (DCM/MeOH, 37:3), mp 182-185° C. (decomposed); UV-vis (DCM) λₘₐₓ (relative intensity) 410(1.0), 505.5 (0.1), 535 (0.07), 585 (0.04), 636 (0.19) nm; Fluorescence (DCM) λₘₐₓ 639 nm (λ excitation=410 nm); ¹H N (270 MHz, 10% DMSO-d₆ in CDCl₃) δ 9.96 (s, 1H, 10-H), 9.92 (s. 1H, NH), 9.42 (s, 1H, 20-H), 9.22 (m, 1H, β-H), 9.02 (d, 1H, J=4.8 Hz, β-H), 9.00 (m, 1H, β-H) 8.92 (m, 1H, β-H), 8.70 (d, 1H, J=4.8 Hz, β-H), 8.53 (m, 1H, β-H), 8.18-7.91 (m, 6H, 5+15-o/m-Ar), 7.33-7.28 (m, 2H, 15-m-Ar), 6.36 (m, 1H, 7-H), 5.96 (m, 1H, 8-H), 4.10 (s, 3H, CH₃), 2.30 (s, 3H, CH₃), −1.74, −2.17 (s, 2H, NH); MALDI-MS m/z 582.6 ([M+H]⁺, 100%). ES-HRMS calcd. for C₃₅H₂₈N₅O₄ ([M+H]⁺) 582.2141, found 582.2135.

High R_{f}-trans bacteriochlorin of Example 12; R_{f}=0.29 (DCM/MeOH, 37:3:1), mp 152-155° C. (decomposed); UV-vis (DCM) λₘₐₓ (relative intensity) 373.5 (1.0) 514 (0.25), 702 (0.49) nm; Fluorescence (DCM) λₘₐₓ 708 nm (λ excitation=514 nm); ¹H NMR (270 MHz, DMSO-d₆) δ 10.27 (s, 1H, NH), 9.16 (s, 2H, 10+20-H), 8.96 (d, 2H, J=4.0 Hz, β-H), 8.24 (d, 2H, J=4.0 Hz, β-H), 7.99-7.89 (m, 6H, 5+15-o/m-Ar), 7.25 (m, 2H, 15-m-Ar), 6.30 (m, 2H, 7+17-H), 6.15 (m, 2H, 8+18-H), 5.63 (m, 2H, OH), 5.32 (m, 2H, OH), 3.99 (s, 3H, CH₃), 2.20 (s, 3H, CH₃), −1.87 (br s, 2H, NH), MALDI-MS m/z 616.3 ([M+H]⁺, 100%). ES-HRMS calcd. for C₃₅H₃₀N₅O₆ ([M+H]⁺) 616.2196, found 616.2192. Low R_{f} cis-bacteriochlorin 13; R_{f}=0.24 (DCM/MeOH, 19:1), mp 148-151° C. (decomposed); UV-vis (DCM) λₘₐₓ (relative intensity) 373.5 (1.0) 514.5 (0.24), 703 (0.50) nm; Fluorescence (DCM) λₘₐₓ 708 nm (λ excitation=514 nm); ¹H N (270 MHz, 20% CD₃OD in CDCl₃) δ 9.20 (s, 2H, 10+20-H), 8.78 (m, 2H, β-H), 8.35 (m, 2H, β-H), 8.05 (m, 2H, 5-o-Ar), 7.89-7.86 (m, 31H, 5+15-o/m-Ar), 7.75 (m, 1H, 15-o-Ar), 7.20-7.17 (m, 2H, 15-m-Ar), 6.25 (m, 2H, 7+17-H), 5.86 (m, 2H, 8+18-H), 4.06 (s, 31H, CH₃), 2.31 (s, 3H, CH₃), (NH's exchanged); MALDI-MS m/z 616.4 ([M+H]⁺, 100%). ES-HRMS calcd. for C₃₅H₃₀N₅O₆ ([M+H]⁺) 616.2196, found 616.2192.

### EXAMPLE 16

### 5-(4-Isothiocyanatophenyl)-10,15,20-tri(4-methylphosphoniumphenyl)-porphyrin and 5-(4-isothiocyanatophenyl)-15-(4-methylphosphoniumphenyl)-porphyrin—General Synthetic Procedure

Boc N-protected 5-(4-aminophenyl)-10,25,20-tri-(4-carbomethoxyphenyl) porphyrin and 5-(4-aminophenyl)-15-(4-carbomethoxyphenyl) porphyrin were synthesised by mixed condensation using Lindsey conditions (Lindsey, J. S., Schreiman, I. C., Hsu, H. C., Kearney, P. C., Marguerettaz, A. M. (1987) J. Org. Chem. 52, 827) or by 2+2 condensation methodology via the appropriately substituted 5-phenyldipyrromethanes as described by Boyle et al (Boyle, R. W., Bruckner, C., Posakony, J., James, B. R., Dolphin, D. (1999) Organic Sytitleses. 76, 287—incorporated herein by reference) respectively. The (4-carbomethoxyphenyl) groups on these porphyrins were then converted to (4-(1-bromomethyl)phenyl) groups using the following standard procedure: the porphyrin (0.2 mmol) was dissolved in dry THE (25 ml) at 0° C. and stirred under argon for 10 minutes. Lithium aluminium hydride (0.7 mmol) was added and the stirring continued for 24 hours. The reaction was monitored by TLC and, when the reaction was complete ethyl acetate (2 ml) was added and the mixture washed with aqueous HCl (0.2 M, 20 ml), saturated sodium bicarbonate solution (30 ml) and finally, brine (20 ml). The organic layer was dried (MgSO₄) and evaporated to dryness to yield the corresponding (4-(1-hydroxymethyl)phenyl) substituted porphyrins, bearing three or one reduced carbomethoxy groups respectively. (4-(1-Hydroxymethyl)phenyl) substituted porphyrins (0.2 mmol) were dissolved in dry chloroform (40 ml) and stirred under argon while triphenylphosphine (1.0 mmol) and carbon tetrabromide (1.6 mmol) were added. The reaction was stirred, in the dark, for 24 hours and then monitored by TLC. Once all the hydroxymethyl groups had been converted to bromomethyl groups the reaction mixture was diluted with dichloromethane (40 ml), washed with saturated sodium bicarbonate (2×20 ml) then brine (2×20 ml) and the organic layer dried (MgSO₄). Removal of solvent by evaporation in vacuo afforded the corresponding bromomethyl porphyrins as purple crystalline solids.

Boc N-protected 5-(aminophenyl)-10,25,20-tri-(4-bromomethylphenyl) porphyrin and 5-(aminophenyl)-15-(4-bromomethylphenyl) porphyrin (0.75 mmol) were dissolved in dry dichloromethane (50 ml) under an atmosphere of argon at 25° C. Triaryl or trialkylphosphine (7.5 mmol) dissolved in dry dichloromethane (10 ml) was injected by syringe and the progress of the reaction was followed by TLC. Upon completion the solvent was evaporated from the reaction in vacuo and the crude product was purified by flash column chromatography (silica; gradient elution: dichloromethane to methanol) to give the required Boc-N-protected-5-(aminophenyl)-methylphosphonium-meso-aryl porphyrins as lustrous purple crystalline solids. The Boc protecting group was removed by dissolution of the porphyrin in chloroform or acetonitrile, depending upon solubility, and addition of trimethylsilyl iodide (5.0 equivalents), after 30 minutes the reaction was quenched with methanol (10 ml). Removal of solvent by evaporation, followed by purification by flash column chromatography (silica; gradient: dichloromethane to methanol) gave the 5-(aminophenyl)-methylphosphonium-meso-aryl porphyrins which were converted to the required mono-4-(isothiocyanatophenyl) compounds by treatment with 1,1′-thiocarbonyldi-2(1H)-pyridone using standard procedures (Clarke, O. J. and Boyle, R. W. (1999 J.C.S. Chem. Commun. 2231).

### EXAMPLE 17

5-(4-Isothiocyanatophenyl)-10,15,20-tri((4-methylphosphono-di-ethoxy)phenyl)-porphyrin and 5-(4-isothiocyanatophenyl)-15-((4-methylphosphono-di-ethoxy)phenyl)-porphyrin—General Synthetic Procedure

Boc N-protected 5-(4-aminophenyl)-10,15,20-tri-(4-bromomethylphenyl) porphyrin and 5-(aminophenyl)-15-(4-bromomethylphenyl) porphyrin (0.75 mmol) were dissolved in a mixture of triethyl phosphite (15 mmol) and dry acetonitrile (50 ml). A reflux condenser was fitted and the reaction was refluxed under argon. The reaction was followed by TLC and upon completion was washed with saturated sodium

bicarbonate (2×20 ml), water (2×20 ml) and brine (2×20 ml). The organic layer was then dried (MgSO₄) and the solvent evaporated in vacuo. The crude product was then purified by flash column chromatography (silica; gradient elution: dichloromethane to ethyl acetate) to give the title compounds as purple crystalline solids. The methylphosphono-di-ethoxy groups were then deprotected to either methylphosphono-mono-ethoxy sodium groups by sonication in aqueous sodium hydroxide for 1 hour followed by reversed phase medium pressure chromatography (C₁₈; gradient elution 0.1% aqueous TFA to methanol) (Boyle, R. W. and van Lier, J. E. (1993) Synlett 351), or to the fully deprotected methylphosphonic acids by treatment with bromotrimethylsilane (2 equivalents per methylphosphono-di-ethoxy group) for 2 hours followed by reversed phase chromatographic purification chromatography (C₁₈; gradient elution 0.1% aqueous TFA to methanol) (McKenna, C. E., Higa, M. T., Cheung, N. H., McKenna, M-C. (1977) 2, 155). Boc deprotection (see above) followed by conversion of the unmasked 4-(aminophenyl) group to its isothiocyanato analogue was performed using standard procedures (Clarke, O. J. and Boyle, R. W. (1999 J.C.S. Chem. Commun. 2231).

### EXAMPLE 18

5-(4-Isothiocyanatophenyl)-10,15,20-tri((4-methylphosphonato-di-ethoxy)phenyl)-porphyrin and 5-(4-isothiocyanatophenyl)-15-((4-methylphosphonato-di-ethoxy)phenyl)-porphyrin—General Synthetic Procedure

Boc N-protected 5-(aminophenyl)-10,15,20-tri-(4-hydroxymethylphenyl) porphyrin and 5-(aminophenyl)-15-(4-hydroxymethylphenyl) porphyrin (0.75 mmol) were dissolved in a mixture of dry dichloromethane and pyridine (4:1) under an atmosphere of argon. Diethyl chlorophosphate (2 equivalents per hydroxymethyl group) was injected and the mixture was stirred for 16 hours. Evaporation of solvent from the reaction mixture followed by chromatographic purification gave the corresponding tri or mono ((4-methylphosphonato-di-ethoxy)phenyl) porphyrins. Treatment with aqueous sodium hydroxide (1M) gave the sodium salts of tri or mono ((4-methylphosphonatoethoxy)phenyl) porphyrins (Boyle, R. W. and van Lier, J. E. (1995) Synthesis 1079). Boc deprotection and generation of the isothiocyanato group were performed as described above.

### EXAMPLE 19

### 5-(4-Isothiocyanatophenyl)-10,15,20-tri((4-methylpyridiniumyl)phenyl)-porphyrin and 5-(4-isothiocyanatophenyl)-15-((4-methylpyridiniumyl)phenyl)-porphyrin—General Synthetic Procedure

Boc N-protected 5-(aminophenyl)-10,25,20-tri-(4-bromomethylphenyl) porphyrin and 5-(aminoophenyl)-15-(4-bromomethylphenyl) porphyrin (0.75 mmol) were dissolved in dichloromethane (50 ml) and pyridine (15 mmol), or substituted pyridine (15 mmol), as required, were added. A reflux condenser was fitted and the reaction was refluxed under argon. The reaction was followed by TLC and, upon completion, was evaporated to dryness in vacuo. The residue was purified by reversed phase medium pressure chromatography (C₁₈; gradient elution 0.1% aqueous TFA to methanol) to yield the N-Boc protected 4-aminophenyl compounds. Deprotection of the aminophenyl group(s) and conversion to the isothiocyanato analogue(s) were conducted using the standard protocols (see above).

### EXAMPLE 20

5-(4-Isothiocyanatophenyl)-15-aryl-10,20-(1,2-dihydroxyethyl)-porphyrin—General Synthetic Procedure

The Fmoc protected 5-(4-aminophenyl)-15-aryl porphyrin (0.8 mmol) was dissolved in dry chloroform (300 ml) under an atmosphere of argon. Freshly recrystallised N-bromosuccinimide (1.8 mmol) in dry chloroform (20 ml) was injected by syringe and the mixture was stirred for 30 min. The solvent was then evaporated in vacuo and the crude product purified by flash column chromatography (silica; gradient elution: hexane to ethyl acetate) to give the required 5,15-dibromo-10,20-diarylporphyrin as a purple crystalline solid. The product was then metallated by refluxing in a chloroform/methanol (9:1) solution of zinc acetate dihydrate (80 mmol). The metallation was followed by visible spectroscopy and, upon completion, was passed through a short column of neutral alumina to remove uncoordinated zinc. The zinc 5,15-dibromo-10,20-diarylporphyrin (0,6 mmol) was dissolved in dry THF to which had been added tetrakis(triphenylphosphine)-palladium(0) (0.6 mmol) and vinyltributyltin (1.4 mmol). The mixture was refluxed under nitrogen for 48 hours after which the solvent was evaporated in vacuo and the residue chromatographed by flash column (silica; gradient elution: dichloromethane to ethyl acetate) to give zinc 5-(Fmoc aminophenyl)-15-aryl-10,20-diethenyl porphyrin as a purple crystalline solid. Zinc 5-(Fmoc aminophenyl)-15-aryl-10,20-diethenyl porphyrin was demetallated by dissolution in a solution of trifluoroacetic acid in dichloromethane (1% v/v) to give 5-(Fmoc aminophenyl)-15-aryl-10,20-diethenyl porphyrin after extracting with water and evaporation of solvent from the organic layer in vacuo. Finally the 10 and 20 ethenyl groups were hydroxylated by osmium tetroxide as described (Sutton J, Fernandez N, Boyle R W (2000) J. Porphyrins and Phthalocyanines 4, 655), however due to the rapidity of the reaction between the ethenyl groups and osmium tetroxide it was possible to selectively hydroxylate these groups by control of reaction time and stoichiometry. In a typical set of conditions the 5-(Fmoc aminophenyl)-15-aryl-10,20-diethenyl porphyrin, when treated with osmium tetroxide (5 equivalents) in 10% pyridine/chloroform for 24-48 hours, gave the desired 5-(Fmoc aminophenyl)-15-aryl-10,20-bis(1,2-dihydroxyethyl) porphyrin, while if longer reaction times (72 hours) and higher molar ratios of osmium tetroxide (7.5 or 10 equivalents) are used under the same conditions 5-(Fmoc aminophenyl)-15-aryl-10,20-bis(1,2-dihydroxyethyl) 7,8-dihydroxychlorin and 5-(Fmoc aminophenyl)-15-aryl-10,20-bis(1,2-dihydroxyethyl) 7,8,17,18-tetrahydroxybacteriochlorin respectively are obtained. All the above products are converted cleanly to the corresponding isothiocyanates upon piperidine mediated deprotection of the amino group (see above) and treatment with 1,1′-thiocarbonyldi-2(1H)-pyridone using standard procedures (Clarke, O. J. and Boyle, R. W. (1999 J.C.S. Chem. Commun. 2231).

### EXAMPLE 21

### 5-(4-Isothiocyanatophenyl)-15-phenyl-10,20-(diaryl)-porphyrins—Synthesis from 5,15-diphenyl Porphyrins by Pd⁰ Mediated Suzuki Coupling

Boc N-protected 5-(aminophenyl)-15-phenyl porphyrin was brominated at the 10 and 20 meso positions as described above. The meso-10,20-dibrominated product (0.75 mmol) was dissolved in dry THF (50 ml) or toluene (50 ml), depending upon the boronic acid used in the coupling reaction, tetrakis-(triphenylphosphine) palladium (0) (0.75 mmol) and anhydrous potassium phosphate (0.75 mmol) were added, a reflux condenser was then fitted to the flask and the whole apparatus was placed under an atmosphere of argon. The required aryl or heterocyclic boronic acid was then added as a solution in the appropriate solvent (10 ml) by injection. The reaction was brought to reflux and followed to completion by TLC. On completion the crude reaction mixture was diluted with dichloromethane (100 ml) and extracted with saturated sodium bicarbonate (2×50 ml), water (2×50 ml) and brine (2×50 ml). The organic phase was dried (Mg SO₄) and concentrated by evaporation in vacuo. Finally, the residue was purified by flash column chromatography (silica; gradient elution: dichloromethane to methanol) to give the Boc N-protected 5-(aminophenyl)-15-phenyl-10,20-(diaryl)-porphyrin as a purple crystalline solid. The Boc protecting group was removed by dissolution of the porphyrin in chloroform or acetonitrile, depending upon solubility, and addition of trimethylsilyl iodide (1.2 equivalents), after 30 minutes the reaction was quenched with methanol (10 ml). Removal of solvent by evaporation followed by purification by flash column chromatography (silica; gradient: dichloromethane to methanol) gave the 5-(aminophenyl)-15-phenyl-10,20-(diaryl)-porphyrin which was converted to the title compound by treatment with 1,1′-thiocarbonyldi-2(1H)-pyridone using standard procedures (Clarke, O. J. and Boyle, R. W. (1999 J.C.S. Chem. Commun. 2231).

### EXAMPLE 22

### 5-(4-Isothiocyanatophenyl)-10,15,20-tri(4-glycosylphenyl)-porphyrin and 5-(4-isothiocyanatophenyl)-15-(4-glycosylphenyl)-porphyrin—General Synthetic Procedure

4-(2′,3′,4′,6′-tetra-O-acetyl-β-D-glucopyranosyloxy)benzaldehyde was condensed with 4-nitrobenzaldehyde and pyrrole using Lindsey conditions (Sol, V., Blais, J. C., Carre, V., Granet, R., Guilloton, M., Spiro, M., Krausz, P. (1999) J. Org. Chem. 64, 4431) and the crude reaction mixture purified by flash column chromatography to give 5-(4-nitrophenyl)-10,15,20-tris[4-(2′,3′,4′,6′-tetra-O-acetyl-β-glucopyranosyloxy)phenyl] porphyrin. Alternatively, 4-(2′,3′,4′,6′-tetra-O-acetyl-β-D-glucopyranosyloxy)benzaldehyde was used to synthesise 5-(4-(2′,3′,4′,6′-tetra-O-acetyl-β-D-glucopyranosyloxy)phenyl) dipyrromethane using the method of Boyle (Boyle, R. W., Bruckner, C., Posakony, J., James, B. R., Dolphin, D. (1999) Organiic Syntheses. 76, 287) which was then condensed to give 5-(4-nitrophenyl)-15,-[4-(2′,3′,4′,6′-tetra-O-acetyl-β-glucopyranosyloxy)phenyl] porphyrin. Reduction of the nitro group of these porphyrins was performed by dissolution in THE and addition of 10% palladium on carbon. Stirring of the mixture under H₂ for 5 hours followed by filtration through Celite and purification by flash column chromatography gave the corresponding amino porphyrins, which were N-protected by reaction with Fmoc chloride (2 equivalents) in anhydrous 1,4-dioxane in the presence of sodium bicarbonate (6 equivalents) under argon. The reaction was monitored by TLC and, upon completion, diluted with dichloromethane and washed with water then brine before drying the organic layer (MgSO₄). Purification by flash column chromatography gave the Fmoc N-protected 5-(4-aminophenyl)-10,15,20-tris[4-(2′,3′,4′,6′-tetra-O-acetyl-p-glucopyranosyloxy)phenyl] porphyrin or 5-(4-aminophenyl)-15,-[4-(2′,3′,4′,6′-tetra-O-acetyl-β-glucopyranosyloxy)phenyl] porphyrin. N and O protecting groups were removed by dissolution of the porphyrin in dichloromethane/morpholine (1:1) and stirring for 1 hour. Removal of solvent by evaporation in vacuo was followed by redissolution of the residue in a mixture of dichloromethane and methanol (4:1). Sodium methanolate in dry methanol(1.5 equivalents per OAc group) was added and the mixture stirred for 1 hour. The fully deprotected porphyrin was recovered by precipitation with hexane. Finally, the 5-(4-aminophenyl) porphyrin was dissolved in dry methanol and 1,1′-thiocarbonyldi-2(1H)-pyridone (2 equivalents) was added. The reaction was stirred under argon for 2 hours and monitored by TLC, upon completion, solvent was evaporated in vacuo'and the crude product was purified by preparative medium pressure reversed phase chromatography (C8;gradient elution: 0.1% aqueous TFA to methanol).

### EXAMPLE 23

### Symmetrical Porphyrin/Chlorin Diol/Bacteriochlorin Tetrol Series 5,15-(3,4,5-Trismethoxyphenyl)porphyrin (A General Procedure)

To a 3 L round bottom flask was added 5-(3,4,5-trismethoxyphenyl)dipyrromethane (1.86 g, 6 mmol), then DCM (1L) under N₂. To this stirred solution was added trimethylorthoformate (48 ml, mmol). A pressure equalizing dropping funnel containing a solution of trichloroacetic acid (23.0 g, mmol) in DCM (500 ml) was then fitted to the flask and the solution added dropwise to the reaction mixture over a period of 10 min. The reaction vessel was covered in aluminium foil to exclude light and allowed to stir under N₂ for a period of 3.5 h. Pyridine was then added to the reaction mixture, rapidly with stirring, and the reaction allowed to stir for a further 16 h. at room temperature under N₂ with the light excluded. The aluminium foil was removed and air was bubbled through the solution for a period of 20 min. After this the reaction was left to stir unstoppered for a further period of 3 h. at room temperature with the aluminium foil removed. The reaction mixture was then concentrated in vacuo to remove DCM and remaining pyridine by evaporator, then high vacuum. The crude reaction mixture was then chromatographed on flash silica-gel (250 ml), (dry loaded on to 50 ml flash silica-gel from DCM and a little methanol to ensure complete solubility) eluting with chloroform. The title compound was obtained as purple crystalline solid (347 mg, 18%); λₘₐₓ/(relative intensity) 410 (1.0), 502 (0.04), 53S (002), 578 (0.015), 630 (0.01) nm; UV-VIS (CH₂Cl₂) (fluorescence) λₘₐₓ=634 nm (λ excitation=408 nm); (270 Mfz, CDCl₃) 10.32(2H, s, 10H, 20-H), 9.40 (4H, d, J=4.8 Hz, β-H), 9.18 (4H, d, J=4.8 Hz, β-H), 7.52 (4H, s, o-Ar), 4.20 (6H, s, CH₃), 4.00 (12H, s, CH₃), −3.10 (2H, br. s, NH); MS (MALDI) m/z=643.4 (100%, M⁺).

7,8-Dihydroxy 5,15-(3,4,5-trismethoxyphenyl)chlorin (A General Procedure)

To a stirred solution of 5,15-(3,4,5-trismethoxyphenyl)porphyrin (50 mg, 77.8 μmol) in HPLC grade chloroform (5.0 ml) was added a solution, in anhydrous pyridine (0.5 ml), of osmium tetroxide (2.5 eq., 0.195 mmol, 49 mg). The reaction vessel was flushed with N₂ and sealed with a lightly greased glass stopper, then covered in aluminium foil to exclude the light and left to stir for 72 h at room temperature. After this period the reaction vessels glass stopper was replaced with a plastic stopper and a continuous stream of hydrogen sulfide as was bubbled through the reaction mixture for 5 min., (a gas outlet needle was attached and allowed excess hydrogen sulfide gas to escape into a series of Drëshel bottles filled with mineral oil and a bleach solution respectively). After this time the reaction mixture was filtered through Celite® and then concentrated in vacuo. Any excess pyridine was removed under high vacuum. The crude reaction mixture was then chromatographed on flash silica-gel (100 ml), (dry loaded on to 20 ml flash silica-gel from DCM and a little methanol to ensure complete solubility) eluting with 1% methanol in DCM. Some starting material was recovered (15%) and the title compound was obtained as browny-purple crystalline solid, (26 mg, 50%); m.p. 170° C. (decomposed); UV-VIS (CH₂Cl₂) λₘₐₓ (relative intensity) 410 (1.0) 504 (0.09), 534 (0.06), 582 (0.04), 636 (0.18) nm; V-VIS (CH₂Cl₂) (fluorescence) λₘₐₓ 639 nm (λ excitation 410 nm); δH(270 MHz, CDCl₃) 9.98 (1R, s, 10-H), 9.42 (1H, s, 20-H), 9.20 (1H, m, β-H), 9.04 (1H, d, J=4.0 Hz, β-H), 8.99 (2H, s, β-H), 8.79 (1H, d, J=4.0 Hz, β-H), 8.66 (1H, m, β-H), 7.45 (1H, d, J=1.6 Hz, 15-o-Ar), 7.42(1H, d, J=1.6 Hz, 15-o-Ar), 7.40 (1H, d, J=1.6 Hz, 5-o-Ar), 7.19 (1H, d, J=1.6 Hz, 15-o-Ar), 6.49 (1H, d, J=7.3 Hz, 7-H), 6.23 (1H, d, J=7.3 Hz, 8-H), 4.17 (3′, s, CH₃), 4.15 (3H, s, CH₃), 4.04 (3H, s, CH₃), 4.00 (3H, s, CH₃), 3.98 (3H, s, CH₃), 3.91(31H, CH₃), −1.80 (1H, br. s, NH), −2.19 (1H, br. s, NH), (OH's not observed); MS (MADI) m/z=677.3 (100%, M⁺); HRMS calcd. for C₃₈H₃₆N₄O₈:676.2533. Found: 676.2587.

7,8,17,18-Tetrahydroxy5,15-(3,4,5-trismethoxyphenyl) Bacteriochlorin (A General Procedure)

To a stirred solution of 5,15-(3,4,5-trismethoxyphenyl)porphyrin (50 mg, 77.8 μmol) in HPLC grade chloroform (5.0 ml) was added a solution, in anhydrous pyridine (0.5 ml), of osmium tetroxide (5.0 eq., 0.39 mmol, 49 mg). The reaction vessel was flushed with N₂ and sealed with a lightly greased glass stopper, then covered in aluminium foil to exclude the light and left to stir for 72 h at room temperature. After this period the reaction vessels glass stopper was replaced with a plastic stopper and a continuous stream of hydrogen sulfide gas was bubbled through the reaction mixture for 5 min., (a gas outlet needle was attached and allowed excess hydrogen sulfide gas to escape into a series of Drëshel bottles filled with mineral oil and a bleach solution respectively). After this time the reaction mixture was filtered through Celite® and then concentrated in vacuo. Any excess pyridine was removed under high vacuum. The crude reaction mixture was then chromatographed on flash silica-gel (100 ml), (dry loaded on to 20 ml flash silica-gel from DCM and a little methanol to ensure complete solubility) eluting initially with 1% methanol in DCM to elute chlorin by-product then 2.5% methanol in DCM to elute the major bacteriochlorin isomer (assumed as trans form Brückner et al (1995) Tetrahedron Lett. 36, 9425). The title compound was obtained as a greeny-pink crystalline solid, (20 mg, 36%); m.p. 135° C. (decomposed); UV-VIS (CH₂Cl₂) λₘₐₓ (relative intensity) 374 (1.0) 512 (0.23), 702 (0.52) nm; UV-VIS (CH₂Cl₂) (fluorescence) λₘₐₓ 708 nm (λ excitation 512 nm); δH(270 MHz, CDCl₃) 9.23 (2H, s, 10-H, 20-H), 8.79 (2H, d, J=3.2 Hz, β-H), 8.44 (2H, d, J=0.2 Hz, β-H), 7.37 (2H, s, 5+15-o-Ar), 7.13 (2H, s, 5+15-o-Ar), 6.31 (2H, d, J=6.5 Hz, 7-H, 17-H), 6.01 (2H, d, J=6.5 Hz, 8-H, 18-h), 4.12 (6H, s, CH₃), 3.92 (6H, s, CH₃), 3.89 (6H, s, CH₃), −1.97 (2H, br. s, NH), (OH's not observed); MS (MALDI) m/z=712.4 (100%, (M+1)⁺), HRMS calcd. for C₃₈H₃₆N₄O₁₀:710.2590. Found: 710.2607.

### EXAMPLE 24

### Unsymmetrical Porphyrin/Chlorin Diol/Bacteriochlorin Tetrol Fluorochrome Sets for Bioconjugation

5-(4-Acetonzidophenyl)-15-(4-methoxyphenyl)porphyrin

The required unsymmetrical diphenylporphyrin was synthesised using the general procedure outlined earlier, but with only slight modification. In this example a mixture of dipyrromethanes were used. Due to the different reactivities of the respective dpyrromethanes, the amounts needed for optimisation of mixed porphyrin were different. For the same scale reaction 5-(4-methoxyphenyl)dipyrromethane (505 mg, 2 mmol) and 5-(4-acetomidophenyl)dipyrromethane (838 mg, 3 mmol) were used. The porphyrin mixture was chromatographed on silica-gel (400 ml), (dry loaded on to 20 ml flash silica-gel from DCM and a little methanol to ensure complete solubility) eluting initially with DCM (1 glass pipette fill of triethylamine was added to 500 ml of eluent to aid elution) to remove 5,15-(methoxyphenyl)porphyrin byproduct. After separation of this component the elution was continued with chloroform to allow 5-(4-Acetomidophenyl)-15-(4-methoxyphenyl)porphyrin collection. The desired porphyrin was obtained as purple crystals; (150 mg, 12%); λₘₐₓ/(relative intensity) 410 (1.0), 502 (0.04), 538 (0.02), 578 (0.015), 630 (0.01) nm; δH(270 MHz, CDCl₃) 10.35 (21, s, 10-H, 20-H), 9.43 (4H, d, J=48 Hz, β-H), 9.14 (41, d, J=4.8 Hz, β-H), 8.65 (2H, d, J=7.2 Hz, 5-m-Ar), 8.22-8.12 (4H, d (overlapping), J=8.1 Hz, 5-o-Ar+15-o-Ar), 7.56 (2H, d, J=8.1 Hz, 15-m-Ar), 4.14 (3H, s, CH₃), −3.00 (2H, br. s, NH); MS (MALDI) m/z 550.3 (100%, M⁺).

5-(4-Aminophenyl)-15-(4-methoxyphenyl)porphyrin

5-(4-Acetomidophenyl)-15-(4-methoxyphenyl)porphyrin (100 mg, 0.182 mmol) was treated with 18% hydrochloric acid (200 ml) and fitted with an air condenser. The green solution was left to warm to 85° C. for a period of 3 h. Prior to cooling, the reaction mixture was concentrated in vacuo (water aspirator; evaporator water bath at 75° C.) to remove excess hydrochloric acid then treated carefully with a solution of triethylamine (50 ml) in DCM. The organic extract was washed with water (100 ml) then saturated brine (100 ml) prior to drying (anhyd. Na₂SO₄), filtering via Buckner funnel and finally concentration in vacuo. The required porphyrin was obtained by chromatography on silica-gel (100 ml), (liquid loaded in 10 ml DCM) eluting with DCM (1 glass pipette full of triethylamine was added to 500 ml of eluent to aid elution). The desired porphyrin was obtained as purple crystals; (150 mg, 12%); λₘₐₓ/(relative intensity) 410 (1.0), 503 (0.045), 538(002), 578 (0.015), 630 (0.005) nm; UV-VIS (CH₂Cl₂) (fluorescence) λₘₐₓ=634 nm (λ excitation=410 nm); δH(270 MHz, CDCl₃) 10.30 (2H, 5, 10-H, 20-H), 9.39 (4H, d, J=4.9 Hz, H), 9.17 (2H, d, J=4.9 Hz, 8-H), 9.10 (2H, d, J=4.9 Hz, β-H), 8.19 (2H, d, J=8.8 Hz, 15-o-Ar), 8.07(2H, d, J=8.1 Hz, 5-o-Ar), 7.35 (2H, d, J=8.8 Hz, 15-m-Ar), 7.14 (2H, d, J=8.1 Hz, 5-m-Ar), 4.13 (3H, s, CH₃), 4.08 (2H, br. s, NH), −3.06 (21, br. s, NH); MS (MALDI) m/z=508.3 (100%, (M+1)⁺).

5-(4-Fluorenomethylaminophenyl)-15-(4-methoxyphenyl)porphyrin

To a stirred solution of 5-(4-aminophenyl)-15-(4-methoxyphenyl)porphyrin (28 mg, 55 μmol) in anhydrous 1,4-dioxane (2.5 ml) was added solid sodium hydrogen carbonate (6 eq., 28 mg, 0.33 mmol). To this mixture was then added a solution of 9-fluorenomethylchloroformate (2 eq., 0.11 mmol, 28.5 mg) in 1,4-dioxane (0.5 ml) under N₂. The reaction flask was covered with aluminium foil to exclude light and stirred at room temperature for a period of 3 h. At this time the reaction had gone to completion (as monitored by TLC). The 1,4-dioxane was removed in-vacuo and the residue partitioned between water (25 ml) and DCM (2×25 ml). The combined organic extracts were washed with saturated brine (25 ml) then dried (anhyd. Na₂SO₄), filtered and concentrated in vacuo. The required porphyrin was obtained by chromatography on silica-gel (100 ml), (dry loaded on to 10 ml flash silica-gel from DCM and a little methanol for solubility) eluting with DCM. The desired porphyrin was obtained as purple crystals; (38 mg, 95%); λₘₐₓ/(relative intensity) 410 (1.0), 505 (0.042), 541 (0.02), 578 (0.015), 633 (0.01) nm; UV-VIS (CH₂Cl₂) (fluorescence) λₘₐₓ=635 nm (λ excitation=410 nm); δH(270 MHz, CDCl₃) 10.35 (2H, s, 10-H, 20-H), 9.69 (1H, br. s, NH), 9.44 (4H, d, J=4.8 Hz, β-H), 9.12 (4H, d, J=4.8 Hz, β-H), 8.20-8.17 (4H, 2×d (overlapping), J=8.1 Hz, 5+15-o-Ar), 7.85 (4H, m, 5+15-m-Ar), 7.76-7.66 (2H, m, fluoreno-Ar), 7.51-7.30 (6H, m, flureno-Ar), 4.69 (2×d, J=7.2 Hz, CH₂), 4.30 (1H, t, J=7.2 Hz, CH), 4.13 (OH, s, CH₃), −3.15 (2H, br. s, NH); MS (MALDI) m/z=731.5 (100%, (M+1)⁺), 508.3 (52%, (M-FMOC+1)⁺).

17,18-Dihydroxy-5-(4-aminophenyl)-15-(4-methoxyphenyl) Chlorin and 7,8-dihydroxy 5-(4-aminophenyl)-1-(4-methoxyphenyl) Chlorin Regioisomers

5-(4-aminophenyl)-15-(4-methoxyphenyl)porphyrin (28 mg, 55.2 mmol)-was converted to a mixture of chlorin diol regioisomers using the general chlorin formation procedure given earlier. The crude reaction mixture was then chromatographed on flash silica-gel (200 ml), (dry loaded on to 20 ml flash silica-gel from DCM and a little methanol to ensure complete solubility) eluting with 1% methanol in DCM to elute first some unreacted starting material then the higher R_{f} chlorin isomer as a browny-purple crystalline solid. The lower R_{f} isomer was obtained by further elution with 2.5% methanol in DCM and gave also a browny-purple crystalline solid.

High R_{f} chlorin regioisomer (2,3-dihydroxy-5-(4-methoxyphenyl)-15-(4-aminophenyl) chlorin, from nOe measurements and JPP paper): (8.5 mg, 30%); m.p. 165° C. (decomposed); UV-VIS (CH₂Cl₂) λₘₐₓ (relative intensity) 401 (0.99), 414 (1.0), 503 (0.08), 535 (0.07), 582 (0.035), 636 (0.22) nm; UV-VIS (CH₂Cl₂) (fluorescence) λₘₐₓ 639 nm (λ excitation 412 nm); δH(270 MHz, 10% MeOH-d₄ in CDCl₃) 9.95 (1H, s, 10-H), 9.42 (1H, s, 20-H), 9.17 (1H, d, J=4.8 Hz, β-H), 9.03 (1H, d, J=4.0 Hz, β-H), 8.97 (2H, s, β-H) 8.78 (1H, d, J=4.8 Hz, β-H), 8.51 (1H, d, J=4.8 Hz, β-AH), 8.05 (2, d, J=8.9 Hz, o-Ar), 7.94 (2H, d, J=8.1 Hz, o′-Ar), 7.25 (2H, d, J=8.9 Hz, m-Ar), 7.12 (2H, d, J=8.1 Hz, m′-Ar), 6.42 (1H, d, J=6.5 Hz, 17-H), 6.03 (1×d, J=6.5 Hz, 18-H), 4.08 (3H, s, CH₃), (NH's exchanged), (OH's not observed).; MS (MALDI) m/z=642.2 (100%, (M+1)⁺).

Low R_{f} chlorin regioisomer (2,3-dihydroxy-5-(4-aminophenyl)-15-(4-methoxyphenyl) chlorin from nOe measurements and JPP paper): (8.5 mg, 30%); m.p. 168° C. (decomposed); UV-VIS (CH₂Cl₂) λₘₐₓ (relative intensity) 401(0.99), 413 (1.0), 507 (0.08), 536 (0.06), 586 (0.025), 637 (0.20) nm; UV-VIS (CH₂Cl₂) (fluorescence) λₘₐₓ 639 nm (λ excitation 412 nm); δH(270 MHz, 10% MeOH-d₄ in CDCl₃) 9.96 (1H, s, 20-H), 9.40 (1H, s, 10-H), 9.18 (1, d, J=4.8 Hz, β-H), 9.05 (1H, d, J=4.8 Hz, β-H), 8.98 (1H, d, J=4.0, β-H) 8.92 (1H, d, J=4.0 Hz, β-H), 8.74 (1H, d, J=4.0 Hz, β-H), 8.58 (1H, d, J=4.0 Hz, O—H), 8.13(11H, d, J=8.9 Hz, o-Ar), 8.08(11H, d, J=8.9 Hz, o-Ar), 7.95 (1H, d, J=8.1 Hz, o′-Ar), 7.79 (1H, d, J=8.1 Hz, o′-Ar), 7.36 (1, d, J=8.9 Hz, m-Ar), 7.30(11H, d, J=8.9 Hz, m-Ar), 7.11(11H, d, J=8.1 Hz, n′-Ar), 7.05(1H, d, J=8.1 Hz, m′-Ar), 6.42 (1H, d, J=6.5 Hz, 7-H), 6.09 (1H, d, J=6.5 Hz, 8-H), 4.11 (3H, s, CH₃), (NH's exchanged), (OH's not observed); MS (MALDI) m/z=642.2 (100%, (M+1)⁺). 17,18-Dihydroxy-5-(4-isothiocyanatophenyl)-15-(4-methoxyphenyl)chlorin (Higher R_{f} Regioisomer)

To a stirred solution of 1,1′-thiocarbonyldi-2(1H)-pyridone (1.07 eq., 7.6 mg, 43.5 mmol) in DCM (25 ml), (dried via passage through an activated alumina column) was added a solution of 17,18-dihydroxy-5-(4-methoxyphenyl)-15-(4-aminophenyl)chlorin (17.5 m&, 23.2 □mol) in DCM (10 ml). The reaction flask was covered with aluminium foil to exclude light and left to stir under N₂ for 2 h, at which time TLC indicated complete loss of starting material. The reaction mixture was then washed with water (2×50 ml) and saturated brine (50 ml) then dried (anhyd. Na₂SO₄). The organic extract was then filtered and concentrated on to 10 ml flash silica-gel and chromatographed on flash silica-cel (100 ml), eluting with 1% methanol in DCM to elute the required isothiocyanato chlorin diol (NB. traces of all TDP must be removed prior to concentration of product from column chromatography otherwise some decomposition to 3 higher R_{f} byproducts occurs. These have not been identified at this time). The title compound was isolated as a browny-purple crystalline solid (17 mg, 90%), m.p. 155,C (decomposed); UV-VIS (CH₂Cl₂) λₘₐₓ (relative intensity) 410 (1.0) 505 (0.09), 534 (0.06), 586 (0.04), 637 (0.18) nm; UV-VIS (CH₂Cl₂) (fluorescence) λₘₐₓ 639 nm (λ excitation 412 nm); 5H(270 MHz, 10% MeOH-d₄ in CDCl₃) 10.0 (1H, s, 10-H), 9.45 (1H, s, 20-H), 9.20 (1H, d, J=4.8 Hz, β-H), 9.06 (1H, d, J=4.0 Hz, β-H), 9.02 (1H, d, J=48 Hz, 8-H) 8.84 (1H, d, J=4.8 Hz, β-H), 8.64 (1H, d, J=4.0 Hz, AH), 8.55 (1H, d, J=4.8 Hz, β-H), 8.21 (1H, d, J=8.1 Hz, o-Ar), 8.15 (1H, d, J=8.1 Hz, o-Ar), 8.05 (1H, d, J=8.9 Hz, o′-Ar) 7.93 (1H, d, J=8.9 Hz, o′-Ar), 7.65 (2H, m, m-Ar), 7.24 (2H, m, m′-Ar), 6.43 (1H, d, J=6.5 Hz, 17-H), 6.04 (1H, d, J=6.5 Hz, 18-H), 4.08 (3H, s, CH₃), (NH's exchanged), (OH's not observed); MS (MALDI) m/z=583.7 (100%, M+); HRMS calcd. for C₃₄H₂₆N₅O₃S: 584.1757. Found: 584.1756 ((M+1)⁺).

7,8,17,18-Tetrahydroxy-5-(4-fluorenomethylaminophenyl)-15-(4-methoxyphenyl) Bacteriochlorin (cis/trans Stereoisomners)

5-(4-Fluorenomethylaminophenyl)-15-(4-methoxyphenyl)porphyrin (35 mg, 48.00 mol) was converted to a mixture of bacteriochlorin stereoisomers using the general bacteriochlorin formation procedure given earlier. The crude reaction mixture was then chromatographed on flash silica-gel (200 ml), (dry loaded on to 20 ml flash silica-gel from DCM and a little methanol to ensure complete solubility) eluting initially with 1% methanol in DCM to elute the higher R_{f} chlorin byproducts, then 2% methanol/DCM to elute separately the two stereoisomeric bacteriochlorins. The higher R_{f}-trans bacteriochlorin isomer was isolated as a pinky-green crystalline solid, (6 mg, 15%); m.p. 142° C. (decomposed); UV-VIS (CH₂Cl₂) λₘₐₓ (relative intensity) 374 (1.0) 512 (0.23), 702 (052) nm; UV-VIS (CH₂Cl₂) (fluorescence) λₘₐₓ 708 nm (λ excitation 512 nm); δH(270 MHz, 10% MeOH-d₄ in CDCl₃) 9.20 (2H, s, 10-H, 20-H), 8.78 (2H, d, J=4.0 Hz, β-H), 8.36 (2H, d, J=4.0 Hz, β-H), 7.95 (2H, m, o-Ar), 7.85 (2H, d, J=7.3 Hz, fluoreno-Ar), 7.79 (2H, m, o′-Ar), 7.65 (2H, m, m′-Ar), 7.47-7.38 (6H, m, fluoreno-Ar), 7.24 (2H, m, m-Ar), 6.27-6.24 (2H, 2×d (overlapping), J=6.5 Hz, 7-H, 17-H), 5.85 (2H, d, J=6.5 Hz, 8-H, 18-H), 4.65 (2H, d, J=7.2 Hz, CH₂), 4.39 (1H, t, J=7.2 Hz, CH), 4.06 (3H, s, CH₃), −1.94 (2H, br. s (partly exchanged), NH), (OH's not observed); MS (MALDI) m/z=800.4 (100%, (M+1)⁺).

The lower R_{f} cis-bacteriochlorin isomer was isolated as a pinky-green crystalline solid, (8.5 mg, 21%); m.p. 148° C. (decomposed); UV-VIS (CH₂Cl₂) λₘₐₓ (relative intensity) 374 (1.0) 512 (0.24), 703 (0.54) nm; UV-VIS (CH₂Cl₂) (fluorescence) λₘₐₓ 708 nm (λ excitation 512 nm); δH(270 MHz, 10% MeOH-d₄ in CDCl₃) 9.12 (2H, s, 10-H, 20-H), 8.76 (2H, d, J=4.8 Hz, β-H), 8.34 (2H, 2×d (overlapping), J=4.8 Hz, β-H), 8.02 (2H, m, o-Ar), 7.76 (2H, d (obscurred), J=8.0 Hz, o′-Ar), 7.83 (2H, d, J=7.3 Hz, fluoreno-Ar), 7.76 (2H, d, J=8.0 Hz, m′-Ar), 7.50-7.38 (6H, m, fluoreno-Ar), 7.24 (2H, m, m-Ar), 6.27-6.23 (2H, 2×d (overlapping), J=6.5 Hz, 7-H, 17-H), 5.85-5.82 (2H, 2×d (overlapping), J=6.5 Hz, 8-H, 18-H), 4.65 (21H, d, J=7.2 Hz, CH₂), 4.39 (1H, t, J=7.2 Hz, CH), 4.05 (3H, s, CH₃), −1.88 (2H, br. s (partly exchanged), NH), (OH's not observed); MS (MALDI) m/z=800.4 (100%, (M+1)⁺).

7,8,17,18-Tetrahydroxy-5-(4-isothiocyanatophenyl)-15-(4-methoxyphenyl) bacteriochlorin (Lower R_{f} Cis Stereoisomer)

A solution of 7,8,17,18-tetrahydroxy-5-(4-fluorenomethyaminophenyl)-15-(4-methoxyphenyl) bacteriochlorin (lower R_{f} cis stereoisomer), (8.5 mg, 10.7 Amok) in 25% methanol in DCM (1.25 ml) was treated with piperidine (50 eq., 53 μl, 0.53 mmol) and left to stir for a period of 3 h. at room temperature under N₂ with the light excluded. The reaction mixture was concentrated in vacuo to remove all traces of piperidine (high vacuum needed). To a stirred solution of 1,1′-thiocarbonyldi-2(1H)-pyridone (1.07 eq., 7.6 mg, 43.5 mmol) in DCM (25 ml), (dried via passage through an activated alumina column) was added a solution of 2,3,12,13-tetrahydroxy-5-(4-aminophenyl)-15-(4-methoxyphenyl)bacteriochlorin (6.1 mg, 10.7 μmol) in DCM (10 ml). The reaction flask was covered with aluminium foil to exclude light and left to stir under N₂ for 2 h, at which time TLC indicated complete loss of starting material. The reaction mixture was then washed with water (2×50 ml) and saturated brine (50 ml) then dried (anhyd. Na₂SO₄). The organic extract was then filtered and concentrated on to 10 ml flash silica-gel and chromatographed on flash silica-gel (100 ml), eluting with 2% methanol in DCM to elute the required isothionato bacteriochlorin tetrol (NB. traces of all TDP must be removed prior to concentration of product from column chromatography otherwise some decomposition occurs). The lower R_{f} cis-bacteriochlorin isomer was isolated as a pinky-green crystalline solid, (5.0 mg, 76%); mp. 132° C. (decomposed); UV-VIS (CH₂Cl₂) λₘₐₓ (relative intensity) 375 (1.0) 516 (0.22), 702 (0.48) nm; UV-VIS (CH₂Cl₂) (fluorescence) λₘₐₓ 709 nm (λ excitation 516 nm); 5H(270 MHz, 10% MeOH-d₄ in CDCl₃) 9.20 (1H, s, meso-H), 9.18 (1H, s, meso′-H), 8.77 (2H, d, J=4.8 Hz, β-H), 8.40 (1H, d, J=4.8 Hz, β-H), 8.34 (1H, d, J=4.8 Hz, β-H), 8.14 (2H, m, o-Ar), 8.05 (2H, m, o′-Ar), 7.42-7.08 (4H, m, 5+15-m-Ar), 6.20 (2H, 2×d (overlapping), J=6.5 Hz, 7-H, 17-H), 5.98 (1H, d, J=6.5 Hz, 8-H), 5.93 (1H, d, J=6.5 Hz, 18-H), 4.04 (3H, s, CH₃), −1.80 (2H, br. s (partly exchanged), NH), (OH's not observed); MS (MALDI) m/z=618.9 (100%, (M+1)⁺), HRMS calcd. for C₃₄H₂₈N₅O₅S: 618.1815. Found: 618.1810 ((M+1)⁺).

Further synthetic protocols and methodology protocols are also described in Sutton et al, Porphyrin Chlorin and Bacteriochlorin Isothiocyanates—Synthesis and Potential Applications in Fluorescence Imaging and Photodynamic Therapy (Journal of Phthalocyanines & Photosensitisers—in press) and in Oliver J Clarke, Isothiocyanato Porphvrins for bioconjugation:synthesis and applications in photochemotherapy and fluorescence imaging (PhD thesis, April 2001, University of Essex); the entire contents of each of which are incorporated herein by reference.

Methodology Description 1: General Bioconjugation Protocol Hexahydroxy PITC+Antibody

A stock solution of hexahydroxy PITC in DMSO was prepared to a molarity of 0.027, this solution was desiccated and stored at 0° C. until required. A solution of antibody was extensively dialysed against sterilised PBS to remove any trace of azide. The dialysed antibody solution was then adjusted to a concentration of 10 mg/mL via centrifugal concentration and separated into 250 μL aliquots.

A 1 M solution of sodium bicarbonate was prepared and adjusted to pH 9.0 with 2 M sodium hydroxide.

To a 250 μL aliquot of antibody was added 30 μL of 1 M sodium bicarbonate. A predetermined volume of hexahydroxy PITC stock solution was then added to give a desired molar ratio (MR) of porphyrin to antibody. For example an MR of 20 was achieved via the addition of 10 μL of stock solution to 250 μL of antibody at 10 mg/mL. In order to maintain a constant concentration of DMSO in the bioconjugation reaction mixture, all aliquots of stock solution were diluted to 25 μL with further portions of DMSO.

TABLE 1.0
Quantities of reagents for bioconjugation

| | Vol. of | [C] of | Vol. of 1 M | Vol. of | | Vol. of | |
|---|---|---|---|---|---|---|---|
| Desired | antibody | antibody | sodium | PITC stock | | extra | |
| MR | solution | solution | bicarbonate | solution | | DMSO | |
| 20 | 250 μL | 10 mg/mL | 30 μL | 10 | μL | l5 | μL |
| 10 | 250 μL | 10 mg/mL | 30 μL | 5 | μL | 20 | μL |
| 5 | 250 μL | 10 mg/mL | 30 μL | 2.5 | μL | 22.5 | μL |
| 2.5 | 250 μL | 10 mg/mL | 30 μL | 1.25 | μL | 23.75 | μL |

Following addition of PITC the bioconjugation reaction was agitated gently for 1 hour at 25° C. After 1 hour the crude bioconjugation reaction mixture was loaded directly onto the top of a prepacked PD10 size exclusion column pre-equilibrated with sterile PBS (25 mL). The column was eluted with sterile PBS. Antibody-porphyrin conjugate was eluted in the first coloured band/fraction. The antibody-porphyrin conjugate concentration following dilution during chromatography was determined as 1.25 mg/mL. The degree of labelling (DOL) of porphyrin to antibody was calculated via standard spectroscopic methods using known constants of molar absorptivity for both porphyrin and protein.

Antibody-porphyrin conjugates were stored, without further concentration, in PBS+azide at 0° C. unless otherwise stated.

N-Methylpyridinium chloride PITC+Antibody

A stock solution of N-methylpyridinium chloride PITC in DMSO was prepared to a molarity of 0.027, this solution was desiccated and stored at 0° C. until required. A solution of antibody was extensively dialysed against sterilised PBS to remove any trace of azide. The dialysed antibody solution was then adjusted to a concentration of 10 mg/mL via centrifugal concentration and separated into 250 μL aliquots.

A 1 M solution of sodium bicarbonate was prepared and adjusted to pH 9.0 with 2 M sodium hydroxide.

To a 250 μL aliquot of antibody was added 250 mL of sterile PBS then 60 μL of 1 M sodium bicarbonate. A predetermined volume of N-methylpyridinium chloride PITC stock solution was then added to give a desired molar ratio (MR) of porphyrin to antibody. For example an MR of 20 was achieved via the addition of 10 μL of stock solution to 500 μL of antibody at 5 mg/mL. In order to maintain a constant concentration of DMSO in the bioconjugation reaction mixture, all aliquots of stock solution were diluted to 25 μL with further portions of DMSO.

TABLE 2.0
Quantities of reagents for bioconjugation

| | Vol. of | [C] of | Vol. of 1 M | Vol. of | | Vol. of | |
|---|---|---|---|---|---|---|---|
| Desired | antibody | antibody | sodium | PITC stock | | extra | |
| MR | solution | solution | bicarbonate | solution | | DMSO | |
| 20 | 500 μL | 5 mg/mL | 60 μL | 10 | μL | 15 | μL |
| 10 | 500 μL | 5 mg/mL | 60 μL | 5 | μL | 20 | μL |
| 5 | 500 μL | 5 mg/mL | 60 μL | 2.5 | μL | 22.5 | μL |
| 2.5 | 500 μL | 5 mg/mL | 60 μL | 1.25 | μL | 23.75 | μL |

Following addition of PITC the bioconjugation reaction was agitated gently for 1 hour at 25° C. After 1 hour the crude bioconjugation reaction mixture was loaded directly onto the top of a prepacked PD10 size exclusion column pre-equilibrated with sterile PBS (25 mL). The column was eluted with sterile PBS. Antibody-porphyrin conjugate was eluted in the first coloured band/fraction. The antibody-porphyrin conjugate concentration following dilution during chromatography was determined as 1.25 mg/mL. The degree of labelling (DOL) of porphyrin to antibody was calculated via standard spectroscopic methods using known constants of molar absorptivity for both porphyrin and protein.

Antibody-porphyrin conjugates were stored, without further concentration, in PBS+azide at 0° C. unless otherwise stated.

Methodology Description 2: Standard Photocytotoxicity

Cells are grown to confluence or appropriate density then washed 2 times with PBS (phosphate buffered saline) to eliminate all trace of FBS (fretal bovine serum). Cell density is adjusted to 1.5×106 cells/ml in medium without FBS and these are then incubated for 1 hour in the dark (37 degrees C., 5% CO₂) with a range of photosensitiser/conjugate concentrations. Post incubation, cells are washed further with medium (without FBS)to eliminate unbound photosensitiser, then resuspended and seeded in 96 wells plates (1×10⁵ cells/well) in quadruplate. Plates are then either irradiated (3.6J/cm2 of filtered red light 0600 nm) or left in the dark as “dark toxicity controls” for the same period of time (14 minutes).Five microliters (5%/well) of FBS is added after the irradiation/dark period and the plates are returned to the incubator overnight. Twenty to 24 hours after treatment, 10 μl of MTT solution (Sigma Thiazolyl blue, 4.8×10⁻⁴M in PBS)is added per well and the plates are returned to the incubator until color develops (between 1 and 4 hours). A solution of acid-alcohol (100 μl/well of 0.04N HCL in isopropanol) is the added and mixed thoroughly to dissolve the dark blue crystals. Plates are then read at 570 nm in a microplate reader and the % cell survival calculated against controls.

Methodology Description 3: Initial Flow Cytometry Chromophore Analysis

The two fluorochromic probes were generated from separate reactions of 2,3,-dihydroxy-5-(4-methoxyphenyl)-15-(4-isothiocyanatophenyl)chlorin (higher R_{f} regioisomer) and 2,3,12,13-tetrahydroxy-5-(4-isothionatophenyl)-15-(4-methoxyphenyl) bacteriochlorin (lower R_{f} cis stereoisomer) with avid in under the standard bioconjugation protocols given earlier. An initial flow experiment has been undertaken utilising these separate avidin conjugates with RAJI cells and biotin monoclonal antibodies (HLA-DR1, L243), (laser excitation 488 nm, collecting emissions at <640 nm (FL2)>670 nm (FL3)). Data indicated that the signals from the DPBC samples were much higher due to good match to emission filter (FL3). Samples containing avidin DPCH or DPBC conjugates with L243 antibodies indicated modest increases in fluorescence compared to controls. Using higher concentrations of avidin-DPCH/DPBC the peak fluorescence increased, which may either be due to the initial concentrations of conjugates being too low to saturate receptors or to a lesser extent to some non-covalent binding. Control samples with avidin-DPCH/DPBC (no antibody) showed some background fluorescence in the absence of L243 antibodies, suggesting that some non-specific binding of the conjugates to the RAJI cells had occurred or that a small quantity of non-covalently bound fluorophore had transferred from the protein to the cell surface. A FITC-avidin control indicated that a slightly higher signal was present in FL2 which appears also in FL3 due to a broad emission band. In the presence of L243 antibody the mean signal increased by 150%. This indicates that non-covalent binding is less significant with FITC-avidin conjugates.

Experiments have been undertaken to determine the level of non-covalent binding of fluorophore to the protein surface (BSA and avidin). ‘Blank’ bioconjugations using mixtures of the unreactive DPCH and DPBC derivatives 2,3-dihydroxy-5-(4-methoxyphenyl)-15-(4-acetomidophenyl)chlorin (higher R_{f} regioisomer) and 2,3,12,13-tetrahydroxy-5-(4-acetomidophenyl)-15-(4-methoxyphenyl) bacteriochlorin (higher R_{f} traits stereoisomer) with both BSA and avidin have been carried out and the resultant protein solutions have been purified by gel filtration (PD-10) as described for the reactive probes described earlier. UV analysis indicated that approximately similar amounts of unreactive probes non-covalently bind to the proteins. For BSA or avidin, 1 unreactive DPCH binds to each protein molecule, whereas DPBC is less than 1 due probably to its increased polarity and non-amphiphilic nature.

Initial studies have been undertaken to remove non-covalently bound fluorophore from the protein (BSA and avidin) using SDS-PAGE. When the ‘blank’ bioconjugation mixtures were subjected to SDS-PAGE separation of all non-covalently bound fluorophore was achieved (UV/fluorescence of a solubilised gel segment at 66000 D for BSA and 16500 D for avidin monomer indicated no signal). Further to these investigations, we have been able to show that fluorophore which is non-covalently bound to BSA (or avidin) transfers to the surface of HeLa cells. When HeLa cells were added to solutions of the non-covalent fluorophore-protein complexes, and incubated for 20 min, fluorescence was removed from the solution with removal of the HeLa cells. This effect was much more marked with 2,3-dihydroxy-5-(4-methoxyphenyl)-15-(4-acetomidophenyl)chlorin (higher R_{f} regioisomer) than with 2,3,12,13-tetrahydroxy-5-(4-acetomidophenyl)-15-(4-methoxyphenyl) bacteriochlorin (higher R_{f} trans stereoisomer). Re-suspension of the cells and measurement of the fluorescence indicated a 10-fold increase in fluorescence in the case of the DPCH, whereas the DPBC only showed a modest increase. These measurements suggest that there is significant fluorescence quenching of both DPCH and DPBC by the protein and that the DPCH's amphiphilic nature has allowed incorporation into the HeLa cell membrane resulting in restoration of almost complete fluorescence. The DPBC, being non-amphiphilic, may complex to the surface of the HeLa cell in a similar manner as it does to the protein resulting in similar fluorescence quenching.

Since the fluorophore conjugates can be purified by SDS-PAGE we have investigated the use of preparative electrophoresis as a technique for removal of non-covalently bound fluorophore. To this end we have used a Centrilutor® micro-electroeluter bought from Millipore. This device has allowed recovery of pure protein fluorophore conjugates from SDS gels.

Methodology Description 4: Elution of Conjugates from SDS PAGE Utilising Micro-Electroeluter

Working in greatly subdued lighting, the SDS-PAGE of the required protein conjugate was cut into small strips and added to the centrilutor sample tubes and the tops closed (no more than half full, 3-4 sample tube used).

The lower buffer chamber of the electroeluter was filled with degassed SDS running buffer up to the level of the first electrode.

3, to 4 Centricon®V centrifugal devices (YM-30 used for BSA conjugates and YM-3 for avidin conjugates) from Millipore were inserted firmly into the holes in the upper buffer chamber rack of the electroeluter from below (with filter membrane lowest) and the vacant holes of the rack were stoppered with stoppers provided, from the underside of the rack.

The upper buffer chamber was placed into the lower buffer chamber with both electrodes aligned on the same side of the electroelutor.

The upper buffer chamber was then filled with degassed SDS running buffer (as before) until all Centricon® unit tops were completely immersed. If no leaks were detected the air bubbles trapped below the Centricon® units were removed via an angled plastic pipette(reinforced with paper clip).

The centrilutor sample tubes were then placed into the top of the Centricon® units, ensuring the sample tube fitted snugly and filled completely with sample buffer (air bubbles were removed as described earlier).

The safety cover of the electroelutor was added and the power supply connected (200 V, 50 mA used).

After a period of 2-3 h. the power supply was removed and the Centricon® filter extracted from the upper buffer chamber of the electroelutor.

The filtrate vial was added to the filter unit and a retentate top added. The excess buffer was then removed by centrifugation at 5000G (BSA) and 7,500G (avidin) for 2 h. Fresh 0.5 M phosphate buffer (pH 7.0) was added to the Centricon® unit and the procedure was repeated to ensure all SDS was removed.

The concentrated purified conjugates were then collected in the retentate vials of the filter units by inversion and centrifugation. Sodium azide (2 M, 20 ml) was added and the conjugates were stored at 4° C.

Methodology Description 5: FACS Conjugate Binding Protocol

Wash flask of cells with phosphate buffered saline (PBS) pH 7.3. Treat with 5 mM EDTA in PBS for 10 min at 37C. Tap flask to dislodge cells, place in 50 mL polypropylene tube and pellet at 400 g 3 min. Resuspend in 10 mL PBS and count cells. Place 2×10⁵ in FACS tube (Falcon 2054) and wash with 1 mL PBS by centrifugation (400 g 3 min) and resuspension by agitation

Block cells in 500 μL 2% Marvel milk powder in PBS, 1% BSA 30 min RT

Wash cells in 1 mL PBS/BSA/Azide centifuge and resuspend pellet (as above)

Add 10 μL appropriate antibody dilution. Incubate on ice 1 h

Wash cells in 1 mL PBS/BSA/Azide centifuge and resuspend pellet (as above)

Add 50 μL Rabbit anti-mouse:FITC (Serotec, 1/100 dilution) and incubate on ice in the dark 1 h

Wash cells in 1 mL PBS/BSA/Azide centiflige (as above) and resuspend pellet in 400 μL PBS/BSA/Azide.

Run samples through FACS machine using CellQuest acquisition software to collect data.

PBS/BSA/AZIDE

250 mL PBS

0.625 g BSA

1.56 mL Sodium Azide (1.6M)

Methodology Description 6: SDS-PAGE
Separating gel

| Component % of gel | 5 | | 20 | |
|---|---|---|---|---|
| Acrylamide/Bis (40% w/v) | 1.67 | mL | 6.66 | mL |
| 1.5 M Tris-HCl (pH 8.8) | 2.5 | mL | 2.5 | mL |
| Water | 5.67 | mL | 0.7 | mL |
| TEMED | 10 | μL | 10 | μL |
| 10% Ammonium persulphate | 50 | μL | 50 | μL |
| SDS | 100 | μL | 100 | μL |

For gradient gel 5-20% a gradient mixer connected to a peristaltic pump
is used.

Stacking gel (3%)

| | |
|---|---|
| Component | mL |

| Acrylamide/Bis (40% w/v) | 1.3 | |
|---|---|---|
| 1 M Tris-HCl (pH 6.8) | 1.25 | |
| Water | 7.4 | |
| TEMED | 20 | μL |
| 10% Ammonium persulphate | 50 | μL |
| SDS | 100 | μL |
| Running buffer | | |
| 0.O25 M Tris, 0.192 M glycine, 0.1% SDS, pH8.3 in water. | | |
| Sample buffer | | |
| 1 M Tris-HCl pH 6.8 | 13 | mL |
| 20% SDS | 6.5 | mL |
| Glycerol | 5.2 | mL |
| 0.5% Bromophenol blue | 0.26 | mL |

Biorad Protean 2 equipment was used in accordance with manufacturer's instructions

Samples (total volume 15-20 μL containing 1-10 μg sample protein) were loaded onto a gel.

Gels were run at 200V for approximately 1 h. Gels were then scanned by light, after which they were stained using Coomassie blue stain and subsequently destained using acetic acid/methanol.

Further Exemplification of the Invention

It has been demonstrated in our original work, described inter alia in Sutton, J., Fernandez, N. and Boyle, R. W. (2000) Functionalised Diphenylchlorins and Bacteriochlorins—Their Synthesis and Bioconjugation for Targeted Photodynamic Therapy and Tumour Cell Imaging. J. Porphyrins and Phthalocyanines 4, 655-658; and Clarke, O. J. and Boyle, R. W. (1999) Isothiocyanatoporphyrins, useful intermediates for the conjugation of porphyrins with biomolecules and solid supports. J.C.S. Chem. Commun. 2231-2232, each of which is incorporated herein by reference, that a set of porphyrin, chlorin and bacteriochlorin molecules can be efficiently conjugated to proteins via a stable thiourea bond, and that these conjugates have potential as fluorescence imaging agents.

As exemplification of the present invention, we now describe the use of this method to form conjugates between monoclonal antibodies having high specificity for human cancer cells, and our set of porphyrin based photosensitisers. Conjugates formed in this way have been assayed for photodynamic activity against the corresponding carcinoma cells, and also for their ability to selectively bind to, and photosensitise, these target cells in the presence of non-target cells. We also demonstrate the specific internalisation of porphyrin-BSA conjugates into HeLa cells.

Our examples utilise 5,10,15-tris(3,5-dihydroxyphenyl)-20-(4-isothiocyanatophenyl) porphyrin (OH6) and 5,10,15-tris(pyridyl)-20-(4-isothiocyanatophenyl)porphyrin (PYR), as we have found from our previous studies that the pattern of hydrophilic substituents around the photoactive porphyrin core of each of these chromophores leads to efficient conjugation with proteins; hydrophilic substituents also minimise non-covalent binding of photosensitiser to protein, often found with more hydrophobic porphyrins. Synthetic protocols for these chromophores are described in Examples 1 and 2 above respectively.

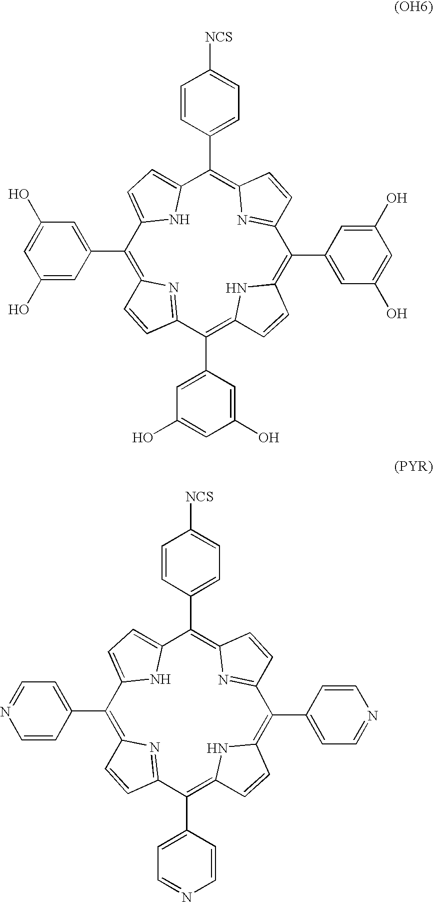

### EXAMPLE 25

### Stable Conjugation to Antibodies

OH6 and PYR were prepared as described in Examples 1 and 2 above respectively. Antibody 17.1A was selected for the bioconjugation procedure. 17.1A is an antibody which reacts specifically with a receptor that is over-expressed on colorectal cancer cells, in particular Colo 320 cells (ECACC, deposit no. 87061205). However, any antibody which reacts against any antigen that is over-expressed on a suitable cell line may be utilised in accordance with the invention. Examples of such antibodies include Ber-EP4 and MOK-31, each of which is commercially available from DAKO Ltd, Ely, Cambridgeshire, and each of which is reactive against an antigen that is over-expressed on epithelial cells.

To increase the buffer pH of the antibody preparation to approximately pH 9, prior to and for the purposes of the bioconjugation procedure, the monoclonal antibody preparation was either buffer-exchanged from a phosphate to an acetate buffer using a Centricon centrifuge or was subjected to dialysis so as to exchange the phosphate buffer for an acetate buffer.

Each of OH6 and PYR was separately conjugated with 17.1A monoclonal antibody in accordance with the method described in Methodology Description 1, to obtain a range of conjugation dilutions having respective MRs of 2.5, 5, 10 and 20.

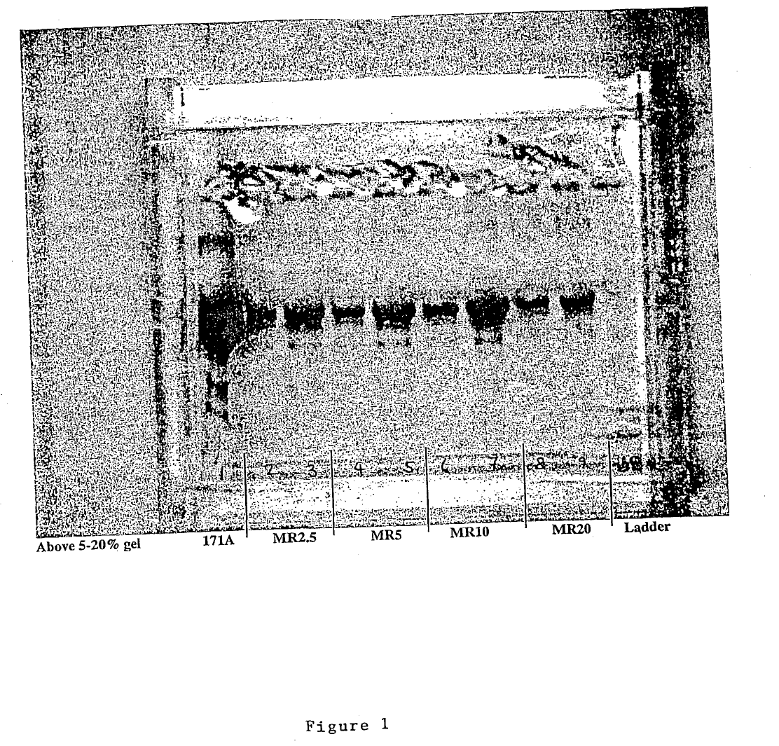

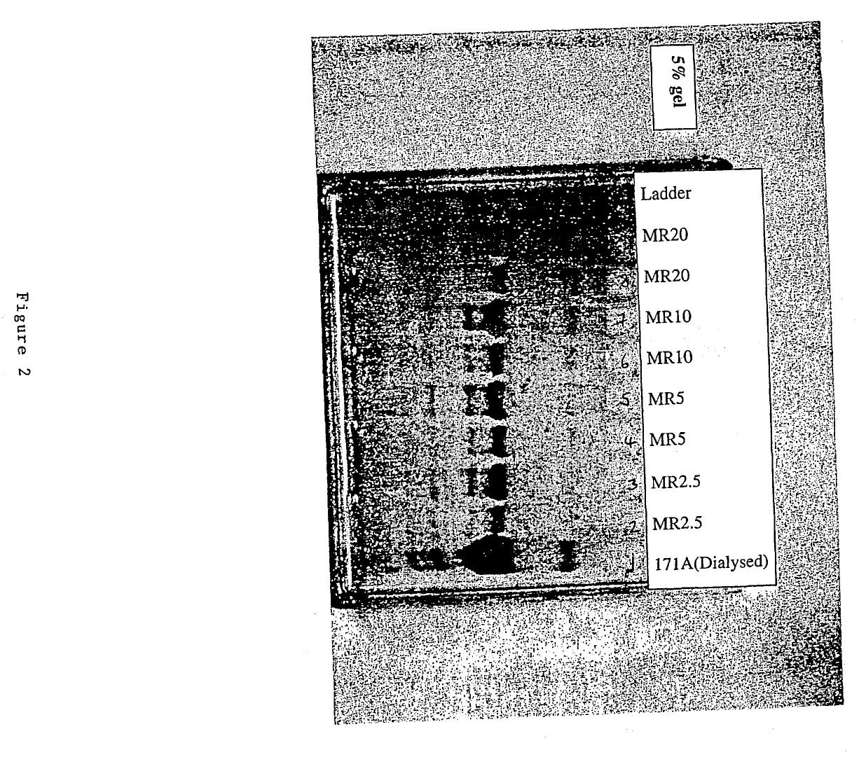

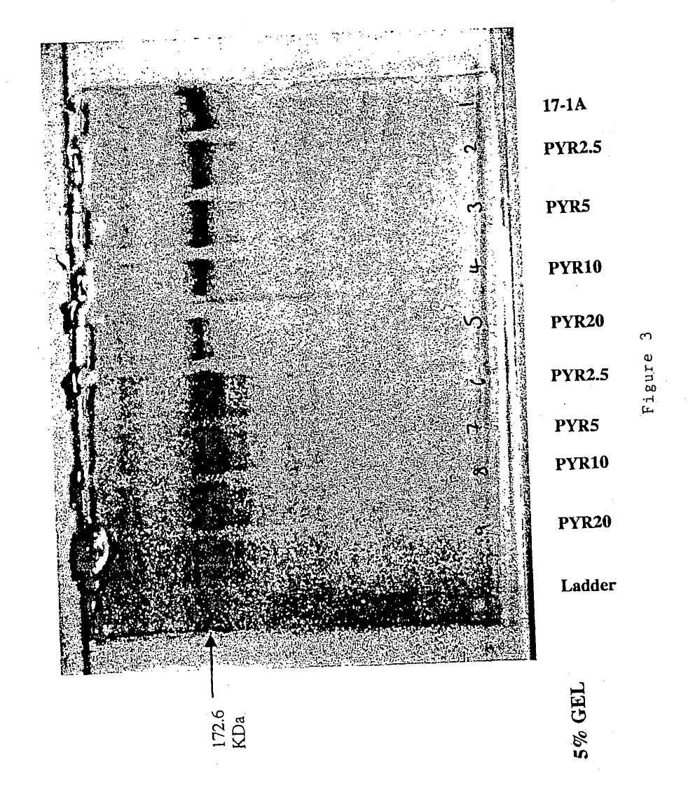

The acetate-buffered antibody preparation and range of conjugation dilutions obtained therefrom were subjected to SDS-PAGE in accordance with the method described in Methodology Description 6. The results are shown in FIGS. 1-3 respectively. FIG. 1 shows a gel loaded with buffer-exchanged 17.1A antibody (lane 1), and buffer-exchanged antibody/OH6 conjugations at MRs 2.5 (lanes 2, 3), 5 (lanes 4, 5), 10 (lanes 6, 7) and 20 (lanes 8, 9) and molecular weight markers (lane 10). FIG. 2 shows a gel loaded with dialysed 17.1A antibody (lane 1), and dialysed antibody/OH6 conjugations at MRs 2.5 (lanes 2, 3), 5 (lanes 4, 5), 10 (lanes 6, 7) and 20 (lanes 8, 9) and molecular weight markers (lane 10). FIG. 3 shows a gel loaded with buffer-exchanged 17.1 A antibody (lane 1), and buffer-exchanged antibody/PYR conjugations at MRs 2.5 (lanes 2, 6), 5 (lanes 3, 7), 10 (lanes 4, 8) and 20 (lanes 5, 9) and molecular weight markers (lane 10).

As seen in these Figures, neither the buffer-exchange nor dialysis procedures disrupt the antibody structure, the light and heavy chains remaining associated with one another and migrating together on each of the gels (lane 1). Conjugation of OH6 and PYR at each of the MRs can also be seen on the gels (lanes 2-9).

### EXAMPLE 26

### FACS Analysis

FACS analyses were run in accordance with Methodology Description 5.

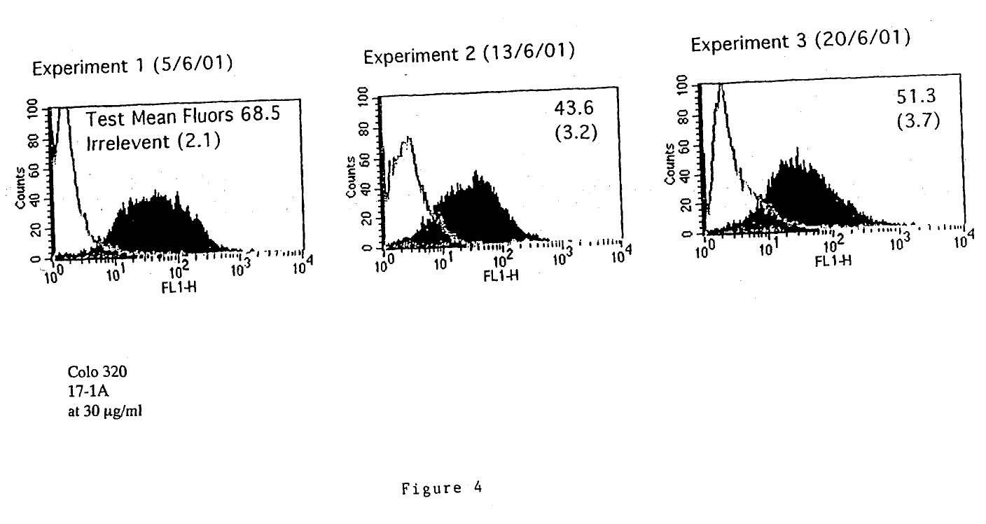

FIG. 4 shows results derived utilising FITC-labelled 17.1A and Colo 320 cells (3 repeats) and indicates that binding of the antibody to the cells has occurred (ie the Colo 320 cells express the antigen specific to 17.1A).

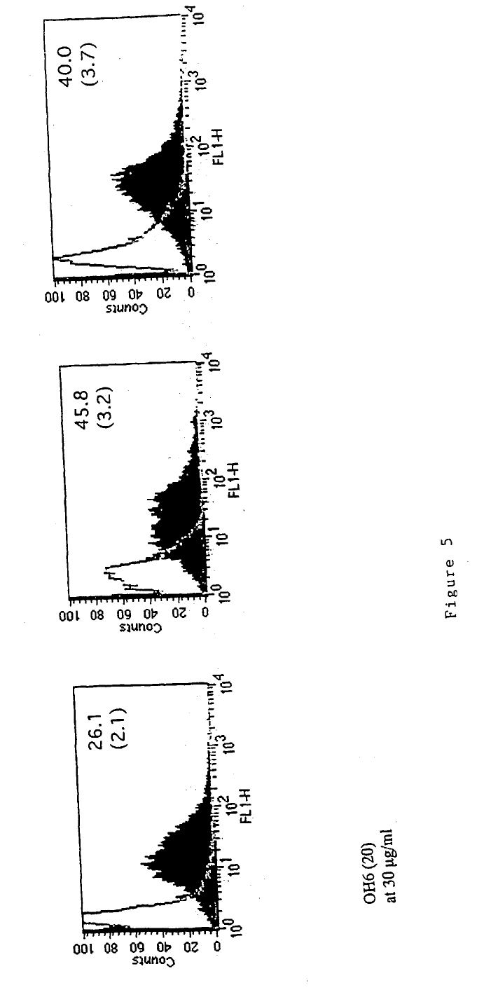

FIG. 5 shows results derived utilising OH6/17.1A conjugate and Colo 320 cells with a FITC-labelled anti-17.1 A antibody for detection (3 repeats) and indicates that the OH6/17.1A conjugate has bound to the cells.

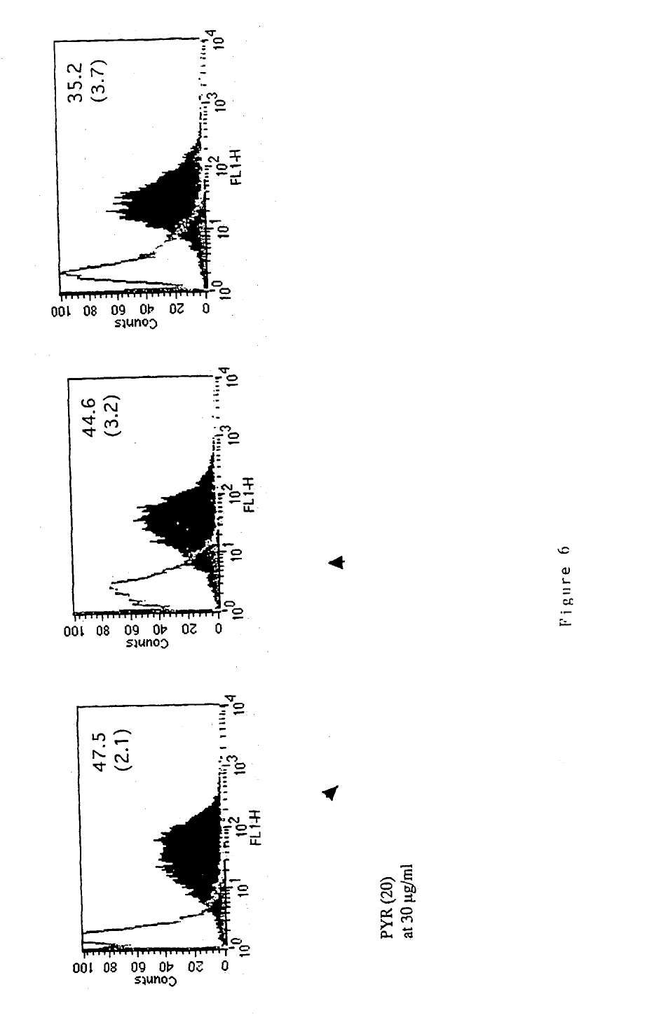

FIG. 6 shows results derived utilising PYk/17.1A conjugate and Colo 320 cells with a FITC-labelled anti-17.1A antibody for detection (3 repeats) and indicates that the PYR/17.1A conjugate has bound to the cells.

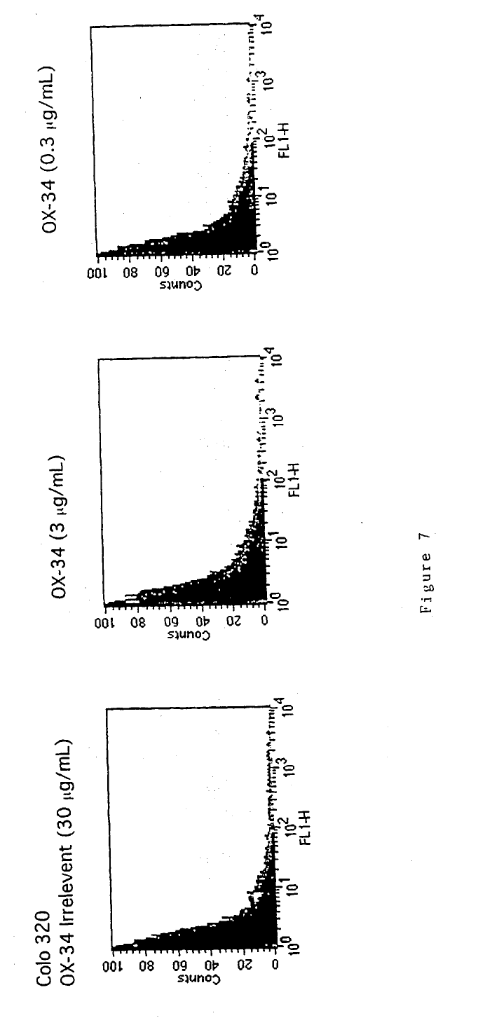

FIG. 7 shows results derived utilising FITC-labelled OX-34 which is an antibody of the same class (IgG2a) as 17.1A but with a different antigen specificity (3 repeats). The results indicate that OX-34 has not bound to the Colo 320 cells and hence that there are no binding sites for OX-34 on Colo 320 cells.

### EXAMPLE 27

### Photocytotoxicity Experiments

Photocytotoxicity tests in accordance with the method described in Methodology Description 2 were performed on Colo 320 cells utilising various antibody conjugates.

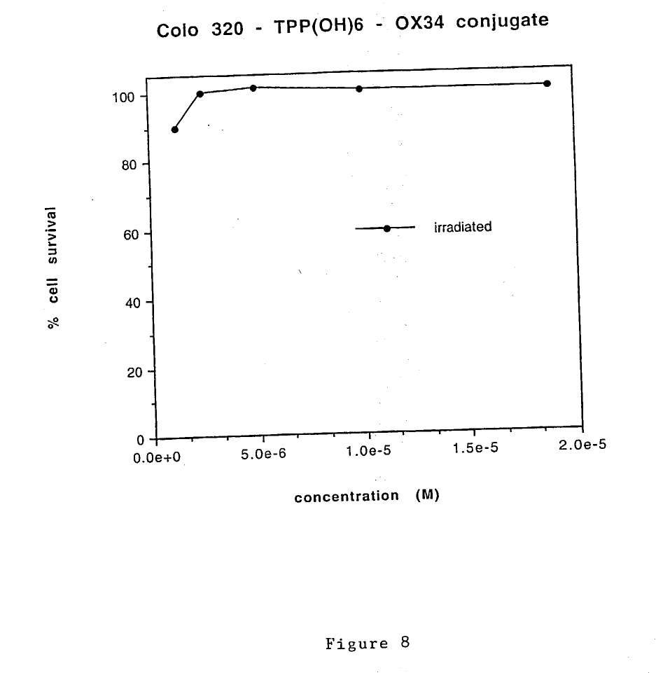

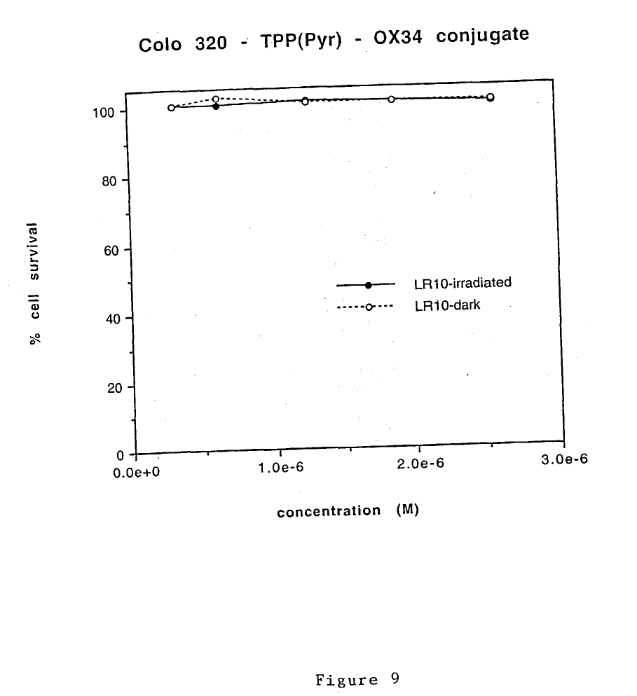

FIGS. 8 and 9 show the results of control experiments performed using OH6/OX-34 and PYR/OX-34 conjugates respectively. As described in Example 16 OX-34 has been found to lack specificity for any antigens expressed on the surface of Colo 320 cells. Accordingly, as expected these control experiments show no photocytotoxicity following irradiation.

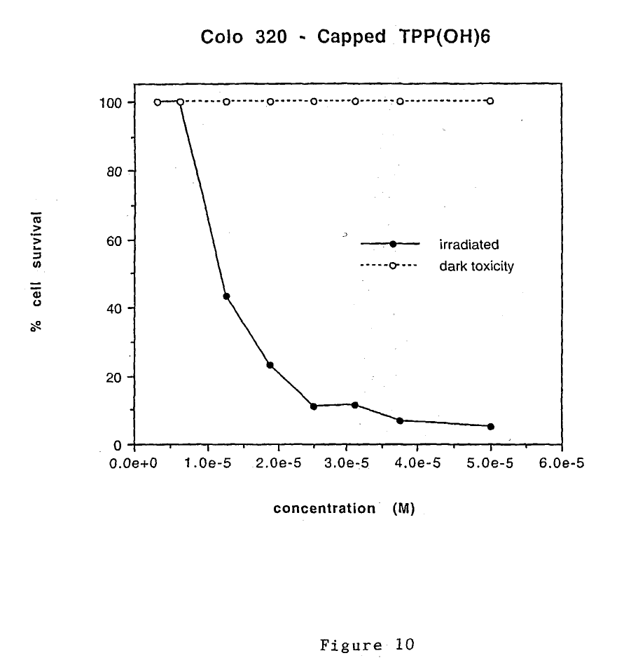

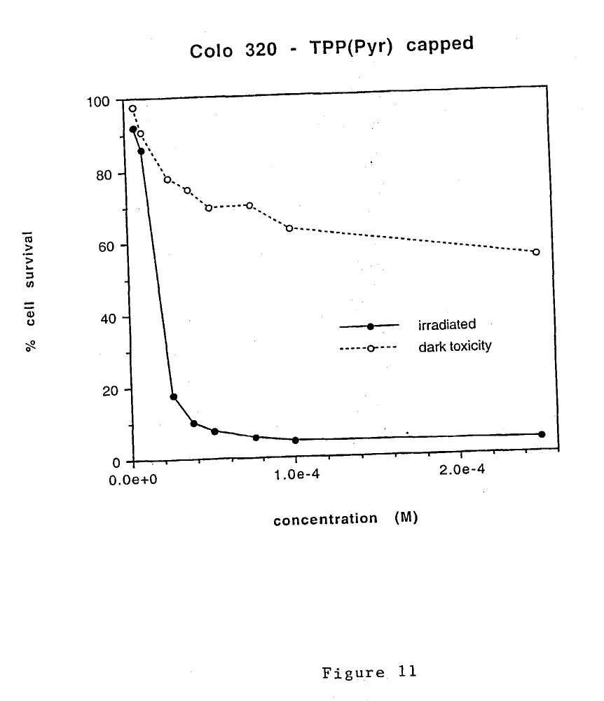

FIGS. 10 and 11 show the results of further control experiments performed using “capped” OH6 and PYR respectively. The “capping” procedure involved reacting the NCS group on each chromophore with propylamine, so as to block serum protein conjugation. FIG. 10 shows no cytotoxicity in the dark, indicating that OH6 is non-toxic to Colo 320 cells. On irradiation, however, some photocytotoxicity is observed, indicating that an amount of the capped OH6 has been transferred to the surface of the Colo 320 cells. FIG. 11 meanwhile shows some cytotoxicity in the dark, suggesting that PYR is to some extent cytotoxic to Colo 320 cells, and increased photocytotoxicity on irradiation, which again indicates that an amount of the capped PYR has been transferred to the surface of the Colo 320 cells.

In the absence of any antibody, transfer of the capped chromophores to the cell membrane is probably attributable to the amphiphilic nature of the capped chromophores, which possess both hydrophilic groups around the porphyrin core and a hydrophobic propylamine “capping” group. This renders them particularly susceptible to becoming embedded in a lipid membrane such as the Colo 320 cell membrane.

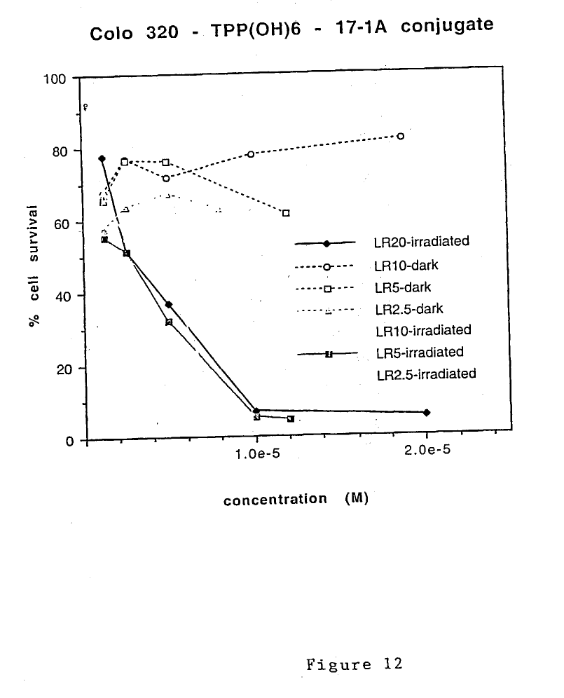

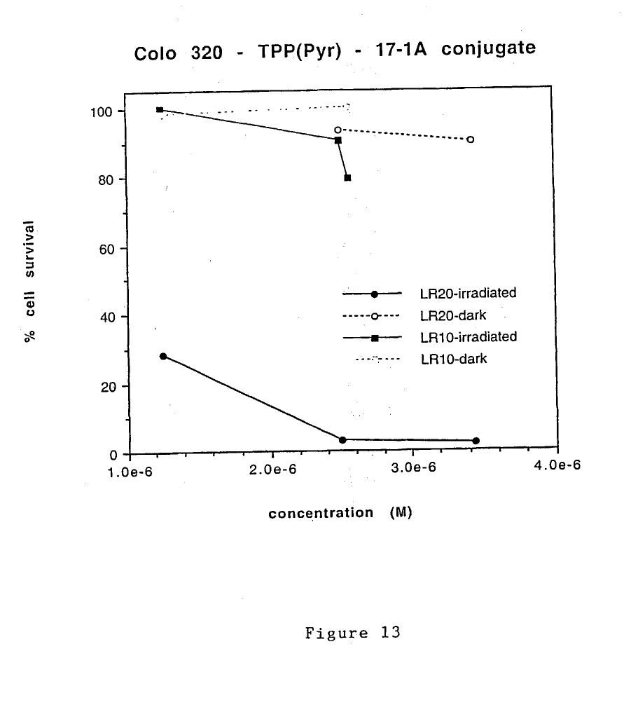

FIGS. 12 and 13 show results obtained using OH6/17.1A and PYR/17.1A conjugates respectively, at various conjugation dilutions (2.5, 5, 10, 20 for OH6/17.1A; 10 and 20 for PYR/17.1A). The results indicate a significant increase in cytotoxicity on irradiation, indicating that the binding of the bioconjugates to the cell surface confers photosensitivity upon the cells. Hence, these species are suitable candidates for PDT.

### EXAMPLE 28

### Photodynamic Therapy In Vivo

Protocols for performing and assessing photodynamic therapy in vivo, utilising the conjugates of the invention, are variously described in R Boyle et al, Br. J. Cancer (1992) 65:813-817; R Boyle et al, Br. J. Cancer (1993) 67:1177-1181; R Boyle et al, Br. J. Cancer (1996) 73:49-53; and Lapointe et al, J. Nuclear Medicine, Vol. 40, No. 5 (May 1999) 876-882; the contents of each of which are incorporated herein by reference.

As described in these papers, tumours may be induced or transplanted into animals such as mice, and the animal may then be injected with a quantity of photosensitiser in accordance with the invention conjugated to an antibody with specificity for an antigen which is specifically expressed or over-expressed on the surface of the tumour cells. Thereafter, the animal may be subjected to irradiation, and the effects on the tumour assessed, qualitatively or metrically, with reference to tumour metabolism (as described in Lapointe et al, J. Nuclear Medicine, Vol. 40, No. 5 (May 1999) 876-882). As described in R Boyle et al, Br. J. Cancer (1996) 73:49-53, the distribution of the photosensitiser in vivo may also be measured, by biodistribution and/or vascular stasis assays.

### EXAMPLE 29

### Confocal Laser Scanning Microscopy

A preliminary examination of the intracellular localisation of a conjugate of 10,15,20-tris(3,5-dihydroxyphenyl)₅-isothiocyanatophenylporphyrin (OH6-NCS) with BSA was carried out using confocal laser scanning microscopy. The readily available epithelial human carcinoma cell line HeLa was selected for incubation with the conjugate. All incubations were performed. in triplicate with sub-confluent cultures of HeLa cells, including a series of control solutions of unlabelled BSA, 10,15,20-tris(3,5-dihydroxyphenyl)5-aminophenylporphyrin porphyrin (OH6-NH2, amino precursor of OH6-NCS), and PBS on its own. Cells were seeded onto coverslips in 35 mm dishes.

Fluorescence images of cells were obtained with a Bio-Rad Radiance2000 confocal laser scanning microscope (Bio-Rad Microscience, Cambridge, Mass.) on an inverted Olympus IX70 microscope using a 60×(NA 1.4) oil immersion objective lens. The illumination source was the 514 nm line from a 25 mW argon ion laser. Porphyrins were visualised with a 514 nm band-pass excitation filter, a 510 nm dichroic mirror, and a 570 nm long-pass emission filter.

Each field of cells was sectioned 3-dimensionally by recording images from a series of focal planes. Movement from one focal plane to another was achieved by a stepper motor attached to the fine focus control of the microscope, the step sizes (in the range 0.5 μm to 1.25 μm) being chosen with regard to the aperture size being used, so that there would be some overlap between adjacent sections. Enough vertical sections were taken so that the tops and bottoms of all the cells in each field would be recorded. Each image collected was the average of four scans at the confocal microscope's normal scan rate. During each imaging session calibration images were taken of: (i) a microscope slide containing medium, in order to measure background levels; (ii) a slide containing ITC porphyrin OH6-NCS dissolved in DMSO; and (iii) a slide bearing only un-probed HeLa cells.

Image data acquisition and remote microscope operation was carried out using the Bio-Rad Lasersharp2000 software. All images were managed using Confocal Assistant version 4.02, (build 101) 1994-1996 Todd Clark Brelje. Artificial colour was applied using standard Bio-Rad look-up tables (LUT).

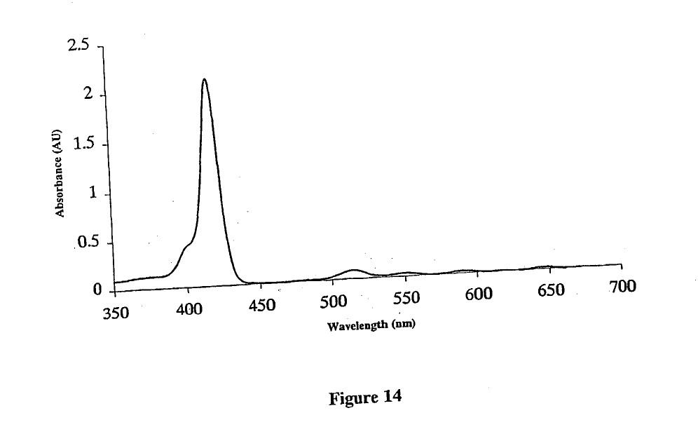

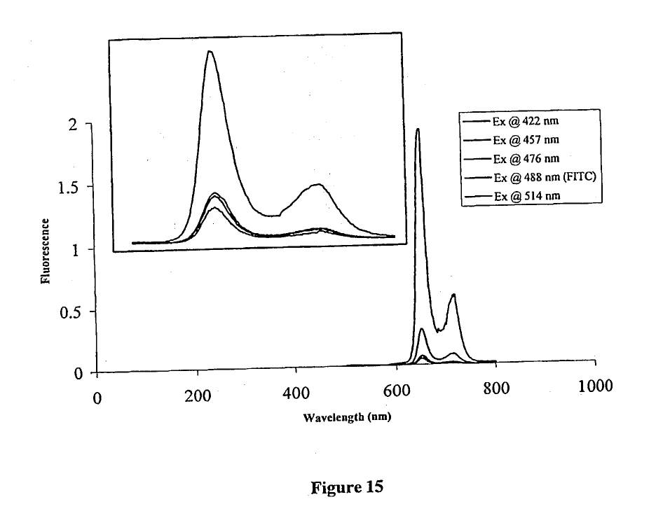

A preliminary evaluation of the fluorescence of OH6-NCS at each of the excitation laser lines available on the CLSM set-up was carried out for a 0.01 mM solution of OH6 in DMSO. FIG. 14 shows the UV-visible spectrum of OH6-NCS identifying its principal absorption bands. Unfortunately, no laser line was available in order to excite OH6-NCS at its Soret band λₘₐₓ. FIG. 15 demonstrates the relative intensities of fluorescence emission for OH6-NCS when excited at 422 nm (optimal), and at the four wavelengths of the argon ion laser, 457, 476, 488, and 514 nm.

It was determined that the intensity of fluorescence emitted by a solution of OH6-NCS when excited at 514 nm was roughly three times greater than fluorescence emission at excitation wavelengths of 457, 476, and 488 nm. The UV-visible absorption spectrum of OH6-NCS showed that the 516 nm argon-ion laser line was the only excitation source compatible with OH6-NCS. The three strongest laser lines, 457, 476, and 488 nm all excited in the region between the Soret and first Q band of OH6-NCS, whereas the 514 line overlapped well with the Q band at 516 nm.

Cell cultures separately incubated with conjugate OH6-NCS-BSA and each of the three controls, were subsequently washed and fixed. Coverslips containing the incubated cells were then cautiously mounted onto standard glass microscope slides ready to be imaged. All four argon-ion laser lines were tested, but, as expected satisfactory resolution of fluorescence could only be achieved using the 514 nm laser line.

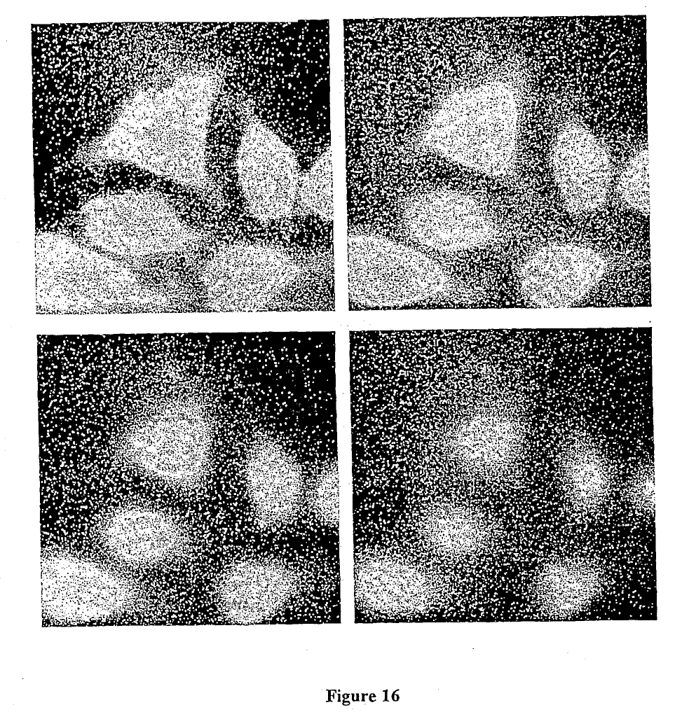

A Z-series fluorescence image of HeLa cells incubated with OH6-NCS-BSA is shown in FIG. 16 (this Figure should be viewed from top left to bottom right). Consecutive sections were scanned with a 2 μM step between each focal plane resolved by the microscope, thus enabling three dimensional visualisation of the localisation of the conjugate within the cell. Clearly the conjugate OH6-NCS-BSA had entered the cell, no studies of the nature of cellular uptake were conducted, however it is most likely that uptake had taken place aria endocytosis. It can be seen that the conjugate has not entered the nucleus and appears to be largely distributed throughout the cytoplasm.

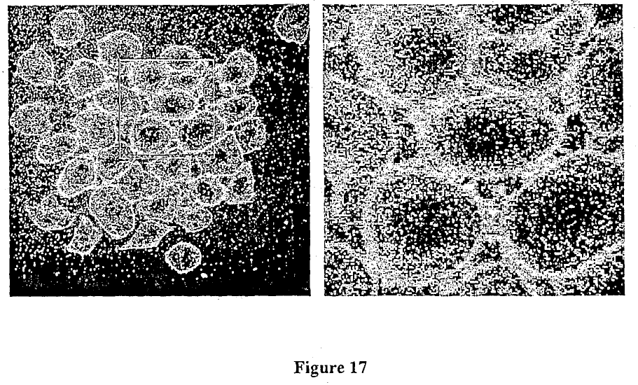

When imaged, cells incubated with the BSA control or the PBS control, showed only very low, barely detectable levels of fluorescence, attributed to normal levels of cellular autofluorescence. The localisation of OH6-NH2 (unconjugated porphyrin control), is shown in FIG. 17, which shows a CLSM image of porphyrin control cells with zoom view. No fluorescence was found to emanate from inside the cells, instead it appeared that the majority of OH6-NH2 had become localised on the plasma membrane. Evidently the BSA component of the conjugate is required in order to facilitate the transport of porphyrin to the interior of the cell.

In summary, it has been shown that the cellular localisation of porphyrin-BSA conjugates, constructed vcia the formation of covalent thiourea linkages, can be imaged using conventional CLSM techniques. Unconjugated porphyrin OH6-NH2 was not found to penetrate the cellular membrane, whereas a significant level of fluorescence was detected from inside cells incubated with OH6-NCS-BSA, indicating good conjugate penetration.

1 A porphyrin chromophore of formula (i) below: 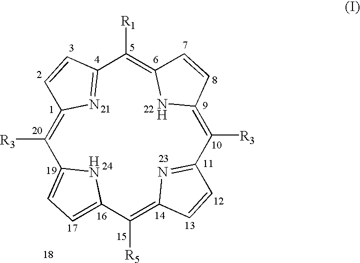
or a chlorin chromophoro of any of formulas (II), (III), (IV), or (V) below: 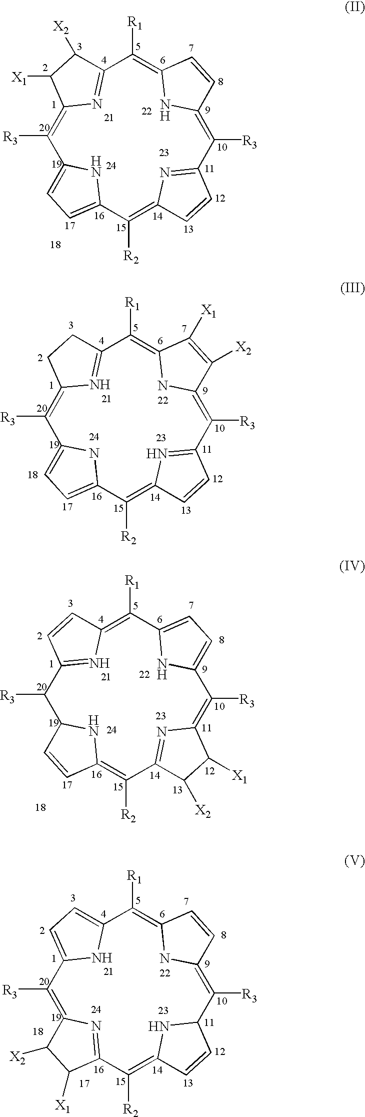
or a bacteriochlorin chromophore of any of formulas (VI) and (VII) below: 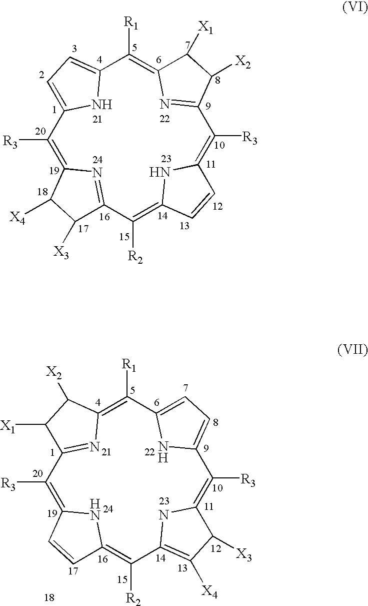
wherein R₁ is an aryl moiety which is linked to a conjugating group Z which is capable of conjugating the chromophore to a polypeptide molecule for delivering said chromophore to a specific biological target in vitro or in vivo; R₂ is a hydrophilic aryl moiety; R₃ is H or a hydrophilic aryl or hydrophilic non-aromatic moiety; and each of X₁, X₂, X₃ and X₄ is independently selected from H, OH, halogen, C₁₋₃ alkyl and OC₁₋₃ alky, or X₁ and X₂ and/or X₃ and X₄ together form a bridging moiety selected from O, CH₂, CH C₁₋₃ alkyl, or C(C₁₋₃ alkyl)₂, such that X₁ and X₂ and/or X₃ and X₄ with the adjacent C—C bond form an epoxide or cyclopropanyl structure; wherein each of said R₁, R₂ and R₃ is optionally further substituted one or more times by —OH, —CN, —NO₂, halogen, -T or —OT, where T is a C₁-C₁₅ alky, cycloalkyl or aryl group or a hydroxylated, halogenated, sulphated, sulphonated or aminated derivative thereof or a carboxylic acid, ester, ether, polyether, amide, aldehyde or ketone derivative thereof.

2 A chromophore as claimed in claim 1, wherein said aryl moiety R₁ comprises a phenyl ring, which phenyl ring is either linked by a single bond to the macrocyclic core of said chromophore or is linked thereto by a C₁₋₆ branched or linear alkyl chain.

3 A chromophore as claimed in any preceding claim, wherein one or both of said R₂ and said R₃ comprises a phenyl ring which is substituted one or more times, preferably at least two times, by one or more hydrophilic substituents which serve to increase the hydrophilicity of said R₂ and/or said R₃.

4 A chromophore as claimed in any of claims 1-3, one or both of said R₂ and said R₃ comprises a heteroaryl ring, such as a quaternised pyridyl (pyridiniumyl) ring, which ring is optionally substituted one or more times, preferably at least two times, by one or more hydrophilic substituents which serve to increase the hydrophilicity of said R₂ and/or said R₃.

5 A chromophore as claimed in claim 3 or claim 4, wherein said one or more hydrophilic substituents are independently selected from hydroxy; alkoxy such as methoxy or ethoxy; C₂-C₁₅ polyethylene glycol; quatenised pyridyl (pyridiniumyl) such as N-methylpyridiniumyl; mono-, di- or poly-saccharide; C₁₋₆alkylsulfonate; a phosphonium group R₄P(R₅)(R₆)(R₇), wherein R₄ is a single bond or C₁₋₆alkyl, and each of R₅, R₆ and R₇ is independently selected from hydrogen, an aryl ring such as a phenyl ring, a heteroaryl ring such as a pyridyl ring, and a C₁₋₆ alkyl chain, which aryl ring, heteroaryl ring or C, alkyl chain is unsubstituted or is substituted one or more times by hydroxy, C₁₋₆ alkyl or alkoxy, aryl, oxo, halogen, nitro, amino or cyano; or a phosphate or phosphonate group R₈OP(O)(OR₉)(OR₁₀) or R₈P(O)(OR₉)(OR₁₀) respectively, wherein R₈ is a single bond or C₁₋₆ alkyl, and each of R₉ and R₁₀ is independently selected from hydrogen and C₁₋₆ alkyl.

6 A chromophore as claimed in any preceding claim, wherein one or both of said R₂ and said R₃ is or are independently selected from m,m-(dihydroxy)phenyl 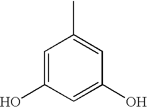
or a PEGylated derivative thereof; m,m,p-(trihydroxy)phenyl 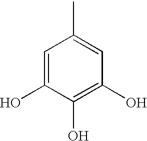
or a PEGylated derivative thereof; o,p,o-(trihydroxy)phenyl 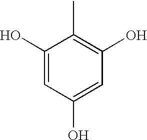
or a PEGylated derivative thereof; m- or p-((C₁₋₆)alkyltriphenylphosphonium)phenyl such as p-(methyltriphenylphosphonium)phenyl 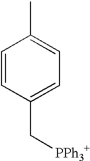
m- or p-(C₁₋₆alkylphosphono-di-alkoxy)phenyl such as p-methylphosphono-di-ethoxy)phenyl 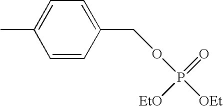
m- or p-(C₁₋₆alkylphosphonato-di-alkoxy)phenyl such as p-methylphosphonato-di-ethoxy)phenyl 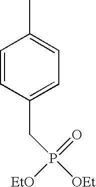
m- or p-(N-methyl-pyridiniumyl)phenyl 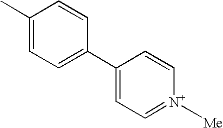
meta- or para- sugar-substituted phenyl such as pentose-, hexose- or disaccharide-substututed phenyl 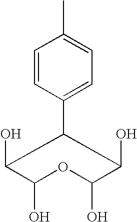
and a quaternised pyridyl (pyridiniumyl) group such as a p-N-(C₁₋₆alkyl)pyridiniumyl group or m-N-(C₁₋₆alkyl)pyridiniumyl group such as m-N-methylpyridiniumyl 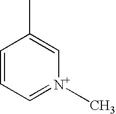
or p-N-methylpyridiniumyl 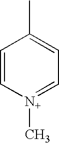
and a zwitterionic group, such as p-N-(C₁₋₆alkylsulfonate)pyridiniumyl or m-N-(C₁₋₆alkylsulfonate)pyridiniumyl; in particular, p-N-(propylsulfonate)pyridiniumyl 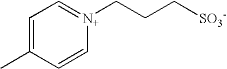
m-N-(propylsulfonate)pyridiniumyl

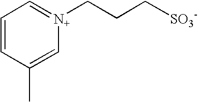

7 A chromophore as claimed in any preceding claim, wherein R₃ is H or is a hydrophilic alkyl moiety, such as a C₁₋₆ alkyl chain which is substituted one or more times by one or more hydrophilic substituents such as hydroxy or C₂₁₅ polyethylene glycol.

8 A chromophore as claimed in any of claims 1-6, wherein R₃ comprises a hydrophilic aryl moiety which is the same as said hydrophilic aryl moiety R₂.

9 A 5,15-diphenylporphyrin, 5,15-diphenylchlorin or 5,15-diphenylbacteriochlorin chromophore, wherein each of the ortho-, meta-, and/or para-positions of each of the 5- and 15-phenyl groups is substituted by a substituent P₁-P₅ and Q₁-Q₅ respectively which is independently H or an inert substituent which in combination with the other substituents P₁-P₅ and Q₁-Q₅ does not substantially impair the fluorescent properties of the chromophore; and the chromophore further comprises a conjugating group Z which is capable of conjugating the chromophore to a polypeptide molecule for delivering said chromophore to a specific biological target in vitro or in vivo.

10 A chromophore as claimed in claim 9, which is selected from the following compounds: 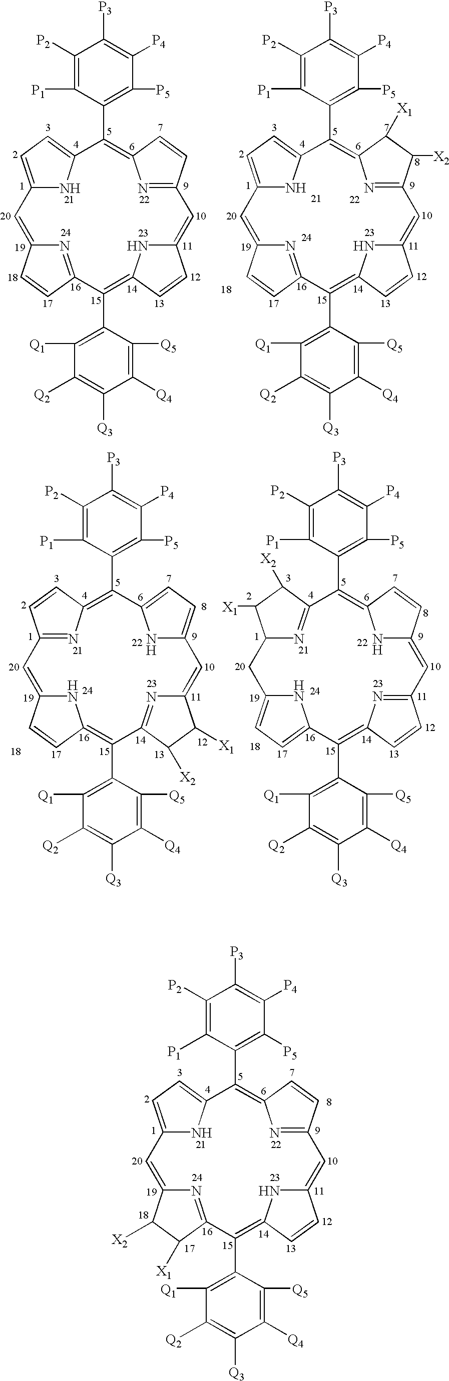 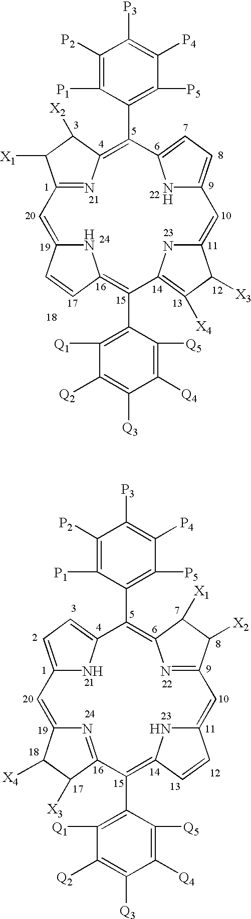
wherein each of X₁, X₂, X₃ and X₄ is independently selected from H, OH, halogen, C₁₋₃ alkyl and OC₁₋₃ alkyl, or X₁ and X₂ and/or X₃ and X₄ together form a bridging moiety selected from O, CH₂, CH C₁₋₃ alkyl or C(C₁₋₃ alkyl)₂, such that X₁ and X₂ and/or X₃ and X₄ with the adjacent C—C bond form an epoxide or cyclopropanyl structure.

11 A chromophore as claimed in claim 9 or claim 10, wherein each of said P₁-P₅ is the same or substantially the same as the corresponding one of said Q₁-Q₅, such that said two primary phenyl rings are symmetrically substituted.

12 A chromophore as claimed in claim 9 or claim 10, wherein one or more of said P₁-P₅ is not the same as the corresponding one of said Q₁-Q₅, such that said two primary phenyl rings are not symmetrically substituted.

13 A chromophore as claimed in any of claims 9-12, wherein said substituents P₁-P₅ and Q₁-Q₅ collectively provide a degree of steric hindrance around the core of said chromophore which is sufficient to reduce the rate of spontaneous oxidation of said chromophore, such that said chromophore is substantially inert in air, but which does not to a substantial extent inhibit selective addition or substitution at the 2, 3, 7, 8, 12, 13, 17 or 18 positions around the core of said chromophore.

14 A chromophore as claimed in any of claims 9-13, wherein one or more of said substituents P₁-P₅ and Q₁-Q₅ comprises H, —OH, —CN, —NO₂, halogen, -T or —OT, where T is a C₁-C₁₅ alkyl, cycloalkyl or aryl group or a hydroxylated, halogenated, sulphated or aminated derivative thereof or a carboxylic acid, ester, ether, polyether, amide, aldehyde or ketone derivative thereof, or a C₃-C₁₂ cycloalkyl and/or aryl ring structure, or between two and six, preferably two-three, fused or linked C₃-C₁₂ cycloalkyl and/or aryl ring structures, each of which ring structures may optionally comprise one or more N, O or S atoms.

15 A chromophore as claimed in any of claims 9-14, wherein one or more of said substituents P₁-P₅ and Q₁-Q₅ consists of a member independently selected from the group consisting of A₁Z₁A₁₄; wherein Z₁ is Z₂, Z₂A₅ or Z₂A₅A₆; A₁ and A₅ are independently selected from —(CA₂A₃)ₙ—, —C(Y)(CA₂A₃)ₙ—, —C(Y)Y′(CA₂A₃)ₙ—, —C(Y)NA₄(CA₂A₃)ₙ—, —NA₄C(Y)(CA₂A₃)ₙ—, —NA₄(CA₂A₃)ₙ, —YC(Y′)(CA₂A₃)ₙ— and —Y(CA₂A₃)ₙ—; n=0−6; Y and Y′ are independently O or S; A₂, A₃ and A₄ are independently H or C₁₋₂ alkyl which is unsubstituted or substituted by one or more fluorines; A₆=−(C₂H₄O)ₘ— or —S(O)ₚ; m=1−12; p=0−2; Z₂ is a single bond or Z₃; Z₃ is selected from Z₄, Z₅ and Z₆, wherein Z₃ is unsubstituted or substituted one or more times by OH, halo, CN, NO₂, A₁A₁₀, A₆A₈, NA₁₀A₁₁, C(Y)A₇, C(Y)Y′A₇, Y(CH₂)_{q}Y′A₇, Y(CH₂)_{q}A₇, C(Y)NA₁₀A₁₁, Y(CH₂)_{q}C(Y′)NA₁₀A₁₁, Y(CH₂)_{q}C(Y′)A₉, NA₁₀C(Y)NA₁₀A₁₁, NA₁₀C(Y)A₁₁, NA₁₀C(Y)Y′A₉, NA₁₀C(Y)Z₆, C(NA₁₀)NA₁₀A₁₁, C(NCN)NA₁₀A₁₁, C(NCN)SA₉, NA₁₀C(NCN)SA₉, NA₁₀C(NCN)NA₁₀A₁₁, NA₁₀S(O)₂A₉, S(O)ᵣA₉, NA₁₀C(Y)C(Y′)NA₁₀A₁₁, NA₁₀C(Y)C(Y′)A₁₀ or Z₆; q=0, 1 or 2; r=0−2; A₇ is independently selected from H and A₉; A₈ is O or A₉; A₉ is C₁₋₄ alkyl which is unsubstituted or substituted by one or more fluorines; A₁₀ is OA₉ or A₁₁; A₁₁ is A₇ or when A₁₀ and A₁₁ are as NA₁₀A₁₁ they may together with the nitrogen form a 5 to 7 membered ring comprising only carbon atoms or carbon atoms and at least one heteroatom selected from O, N and S; Z₄ is C₆₋₁₂ aryl or aryloxyC₁₋₃alkyl; Z₅ is selected from furanyl, tetrahydrofuranyl, indanyl, indenyl, tetrahydropyranyl, pyranyl, thiopyranyl, tetrahydrothiopyranyl, tetrahydrothienyl, thienyl, C₃₋₈ cycloalkyl or C₄₋₈ cycloalkyl containing one or two unsaturated bonds, and C₇₋₁₁ polycycloalkyl; Z₆ is selected from N-azolyl, dioxadiazinyl, dioxadiazolyl, dioxanyl, 2-N-dioxatriazinyl, dioxazinyl, N-dioxazolyl, dioxolyl, dithiadiazinyl, dithiadiazolyl, N-dithiatriazinyl, dithiazinyl, N-dithiazolyl, 1-N-imidazolyl, N-morpholinyl, pyrollyl, tetrazolyl, thiazolyl, triazolyl, oxazinyl, oxazolyl, naphthydrinyl, oxadiazinyl, oxadiazolyl, oxatetrazinyl, oxatriazinyl, oxatriazolyl, oxazinyl, oxazolyl, pentazinyl, phthalazinyl, N-piperidinyl, N,N-piperazinyl, 1-N-pyrazolyl, pyridazinyl, pyridinyl, pyrimidinyl, tetrathiazinyl, tetrazinyl, 1-N-tetrazolyl, tetroxazinyl, thiadiazinyl, thiadiazoyl, thiatetrazinyl, thiatriazinyl, thiatriazoiyl, thiazolyl, triazinyl, 1-N-triazolyl, trioxadazinyl, trioxanyl, trioxazinyl, trioxazolyl, trithiadiazinyl, trithiazinyl, trithiadiazolyl; wherein Z₄, Z₅ or Z₆ may be fused to one or more other members selected independently from Z₄, Z₅ and Z₆; A₁₄ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl or heterocyclic moiety is unsubstituted or substituted by 1 to 3 methyl groups, one ethyl group, or a hydroxyl group.

16 A chromophore as claimed in any of claims 9-15, wherein one of said P₁-P₅ and said Q₁-Q₅ is a conjugating substituent which comprises said conjugating group Z.

17 A chromophore as claimed in claim 16, wherein said conjugating substituent consists of a member selected from the group consisting of A₁Z₁Z; wherein Z₁ is Z₂, Z₂A₅ or Z₂A₅A₆; A₁ and A₅ are independently selected from —(CA₂A₃)ₙ—, —C(Y)(CA₂A₃)ₙ—, —C(Y)Y′(CA₂A₃)ₙ—, —C(Y)NA₄(CA₂A₃)ₙ—, —NA₄C(Y)(CA₂A₃)ₙ—, —NA₄(CA₂A₃)ₙ, —YC(Y′)(CA₂A₃)ₙ— and —Y(CA₂A₃)ₙ—; n=0−6; Y and Y′ are independently O or S; A₂, A₃ and A₄ are independently H or C₁₋₂ alkyl which is unsubstituted or substituted by one or more fluorines; A₆=— (C₂H₄O)ₘ or —S(O)ₚ; m=1−12; p=0−2; Z₂ is a single bond or Z₃; Z₃ is selected from Z₄, Z₅ and Z₆, wherein Z₃ is unsubstituted or substituted one or more times by OH, halo, CN, NO₂, A₁A₁₀, A₆A₈, NA₁₀A₁₁, C(Y)A₇, C(Y)Y′A₇, Y(CH₂)_{q}Y′A₇, Y(CH₂)_{q}A₇, C(Y)NA₁₀A₁₁, Y(CH₂)_{q}C(Y′)NA₁₀A₁₁Y(CH₂)_{q}C(Y′)A₉, NA₁₀C(Y)NA₁₀A₁₁, NA₁₀C(Y)A₁₁, NA₁₀C(Y)Y′A₉, NA₁₀C(Y)Z₆, C(NA₁₀)NA₁₀A₁₁, C(NCN)NA₁₀A₁₁, C(NCN)SA₉, NA₁₀C(NCN)SA₉, NA₁₀C(NCN)NA₁₀A₁₁, NA₁₀S(O)₂A₉, S(O)ᵣA₉, NA₁₀C(Y)C(Y′)NA₁₀A₁₁, NA₁₀C(Y)C(Y′)A₁₀ or Z₆; q=0, 1 or 2; r=0−2; A₇ is independently selected from H and A₉; A₈ is O or A₉; A₉ is C₁₋₁₄ alkyl which is unsubstituted or substituted by one or more fluorines; A₁₀ is OA₉ or A₁₁; A₁₁ is A₇ or when A₁₀ and A₁₁ are as NA₁₀A₁₁ they may together with the nitrogen form a 5 to 7 membered ring comprising only carbon atoms or carbon atoms and at least one heteroatom selected from O, N and S; Z₄ is C₆₋₁₂ aryl or aryloxyC₁₋₃alkyl; Z₅ is selected from furanyl, tetrahydrofuranyl, indanyl, indenyl, tetrahydropyranyl, pyranyl, thiopyranyl, tetrahydrothiopyranyl, tetrahydrothienyl, thienyl, C₃₋₈ cycloalkyl or C₄₋₈ cycloalkyl containing one or two unsaturated bonds, and C₇₋₁₁ polycycloalkyl; Z₆ is selected from N-azolyl, dioxadiazinyl, dioxadiazolyl, dioxanyl, 2-N-dioxatriazinyl, dioxazinyl, N-dioxazolyl, dioxolyl, dithiadiazinyl, dithiadiazolyl, N-dithiatriazinyl, dithiazinyl, N-dithiazolyl, 1-N-imidazolyl, N-morpholinyl, pyrollyl, tetrazolyl, thiazolyl, triazolyl, oxazinyl, oxazolyl, naphthydrinyl, oxadiazinyl, oxadiazolyl, oxatetrazinyl, oxatriazinyl, oxatriazolyl, oxazinyl, oxazolyl, pentazinyl, phthalazinyl, N-piperidinyl, N,N-piperazinyl, 1-N-pyrazolyl, pyridazinyl, pyridinyl, pyrimidinyl, tetrathiazinyl, tetrazinyl, 1-N-tetrazolyl, tetroxazinyl, thiadiazinyl, thiadiazoyl, thiatetrazinyl, thiatriazinyl, thiatriazolyl, thiazolyl, triazinyl, 1-N-triazolyl, trioxadazinyl, trioxanyl, trioxazinyl, trioxazolyl, trithiadiazinyl, trithiazinyl, trithiadiazolyl; wherein Z₄, Z₅ or Z₆ may be fused to one or more other members selected independently from Z₄, Z₅ and Z₆.

18 A chromophore as claimed in any of claims 9-17, which has a structure set out as (x), (y) or (z) below: 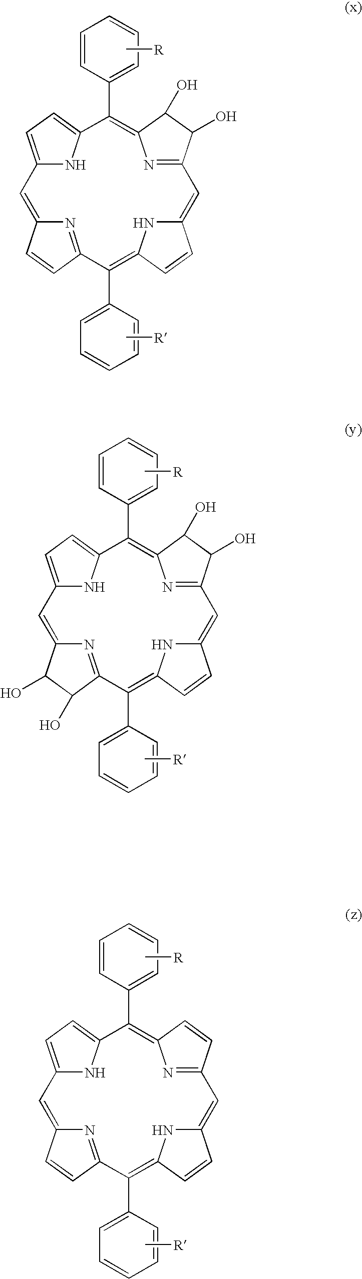
wherein R and R′ may be any of the following combinations:

| R | R′ |
|---|---|
| 4-H | 4-NCS |
| 4-Me | 4-NCS |
| 4-Br | 4-NCS |
| 4-CO₂Me | 4-NCS |
| 3,4,5-tris(OMe) | 4-NCS |
| 4-NCS | 4-OMe |
| 4-NCS | 4-Me |
| 4-NCS | 4-CO₂Me |
| 4-NCS | 4-Br |
| 4-NCS | 4-CN |
| 4-NCS | 4-CO₂Me |

19 A chromophore as claimed in any preceding claim, wherein each or some of X₁-X₄ is H or OH.

20 A chromophore as claimed in any preceding claim, wherein said conjugating group Z comprises a bonding group which is capable of bonding covalently to a polypeptide molecule; such as an isocyanate, isothiocyanate, or NHS ester group; or —NH₂, —NH(C₁₋₆ alkyl), maleamide, iodoacetamide, ketone or aldehyde.

21 A chromophore as claimed in claim 20, wherein said conjugating group Z comprises a linking moiety having a relatively high degree of inflexibility and/or steric hindrance, which linking moiety is adapted to link said bonding group to the macrocyclic core of said chromophore.

22 A set of fluorochromic markers for multicolour fluorochromic analysis, comprising at least two chromophores selected from the group consisting of a porphyrin chromophore, a chlorin chromophore and a bacteriochlorin chromophore, each of which chromophores comprises the same porphyrin skeleton, each of which chromophores comprises one or more substituents on said porphyrin skeleton, one of which substituents is a conjugating substituent L comprising a conjugating group Z, wherein Z is a conjugating group capable of conjugating each of said chromophores to a polypeptide molecule for delivering each chromophore to one of a plurality of different specific biological targets.

23 A set of chromophores as claimed in claim 22, comprising two or more of a porphyrin in accordance with any of claims 1-21, the corresponding chlorin, and the corresponding bacteriochlorin.

24 A chromophore as claimed in any of claims 1-21 or a set as claimed in claim 22 or claim 23, wherein said conjugating group Z is conjugated to a binding protein which is adapted to bind specifically to said biological target; or is conjugated to a bridging polypeptide which is adapted to bind to a complementary bridging polypeptide so as to couple said chromophore to said complementary bridging polypeptide.

25 A chromophore or a set as claimed in claim 24, wherein said bridging polypeptide is bound to said complementary bridging polypeptide, and said complementary bridging polypeptide comprises or is coupled to or fused with a binding protein which is adapted to bind specifically to said biological target.

26 A kit of chromophores comprising a chromophore or set of chromophores in accordance with any preceding claim, wherein said or each chromophore is conjugated to a bridging polypeptide that is adapted to bind to a complementary bridging polypeptide so as to couple the chromophore to said complementary bridging polypeptide; and a construct or plurality of constructs each of which comprises said complementary bridging polypeptide fused or coupled to a binding protein which is adapted to bind specifically to said biological target; the arrangement being such that said chromophore or each chromophore in the kit is adapted to bind to a different construct in the kit with specificity for said specific biological target, so as to link said or each chromophore to a binding protein with specificity for said specific biological target.

27 A chromophore, set of chromophores or kit of chromophores in accordance with any of claims 24-26, wherein said binding protein comprises an antibody such as a monoclonal or polyclonal antibody or a fragment thereof with specificity for a target specific molecule on the surface of said biological target.

28 A chromophore or set of kit of chromophores as claimed in claim 27, wherein said antibody is a phage antibody, that is an antibody expressed on the surface of a bacteriophage.

29 A chromophore, set of chromophores or kit of chromophores in accordance with any of claims 24-26, wherein said binding protein comprises a protein which is adapted to bind to one or more cell surface molecules or receptors, such as a serum albumin protein, or a low density lipoprotein, such as a fatty acid chain, which is adapted for insertion into a cell membrane.

30 A chromophore or set of kit of chromophores as claimed in any of claims 24-29, wherein said bridging polypeptide comprises calmodulin and said complementary bridging polypeptide comprises calmodulin binding peptide, or vice versa; or said bridging polypeptide comprises avidin or streptavidin and said complementary bridging polypeptide comprises biotin; or vice versa.

31 A kit of chromophores as claimed in claim 30, wherein said or each chromophore is conjugated to avidin, and said or each construct comprises a biotinylated monoclonal antibody with specificity for a target specific molecule on the surface of said biological target.

32 A method for attaching a chromophore in accordance with any of claims 1-30 to said specific biological target or targets; comprising the steps of providing a kit in accordance with any of claims 26-31, and introducing the components of said kit into the vicinity of said specific biological target or targets, under conditions suitable for enabling the binding of said or each binding protein to said specific biological target or targets.

33 A chromophore or set of kit of chromophores as claimed in any preceding claim, wherein said specific biological target is a cell or a membrane, such as a cancer cell, a tumour cell, a cell infected with HIV or with any other microbe or virus, a cell responsible for detrimental activity in auto-immune disease, a foreign or diseased cell, or any other such cell.

34 A method for fluorescence-activated sorting of target cells from a mixture of cells, comprising the step of attaching to said target cells a chromophore in accordance with any of claims 1-10 or a set of chromophores in accordance with any of claims 22-30, illuminating said mixture of cells so as to cause fluorescence of one or more of said chromophores attached to said target cells, imparting a charge to the fluorescing cells, and passing said mixture of cells through a polarised environment so as to cause or allow said charged cells to be separated from said mixture.

35 A method for the visualisation and/or counting of a plurality of target cells, said target cells including cells of two or three different cell types, comprising the steps of providing a chromophore set in accordance with any of claims 22-30, which chromophore set comprises two or three chromophores each of which is adapted to be delivered to a different one of said cell types; attaching said chromophores in the set to said target cells; illuminating said target cells so as to cause the emission of fluorescence by said chromophores; detecting the fluorescent emission bands produced by each of said chromophores; and optionally measuring for each of said bands the area under an emission/wavelength curve, so as to obtain a measure of the number of fluorescent cells of each respective cell type.

36 A method for causing the death of a target cell, comprising the step of attaching a chromophore in accordance with any of claims 1-21 to said cell and illuminating said cell so as to cause the production of singlet oxygen in the vicinity of said cell, thereby causing the death of the cell.

37 A method for treating a disease or disorder which is characterised by the presence in the body of diseased or undesired cells, such as tumours, cancers, viral infections such as HIV infection, or autoimmune disorders such as rheumatoid arthritis or multiple sclerosis, comprising the step of administering to a patient in need thereof an effective amount of a chromophore in accordance with any of claims 1-21, which chromophore is adapted to be targeted to a target cell specific molecule on the surface of said diseased or undesired cells for attachment thereto, such that the chromophore is caused to be attached to said cells, and illuminating said cells with light so as to cause the production of singlet oxygen in the vicinity of said cells, thereby killing said cells.

38 A pharmaceutical composition for administration to a patient for the treatment of a disease or disorder which is characterised by the presence in the body of diseased or undesired cells, such as tumours, cancers, viral infections such as HIV infection, or autoimmune disorders such as rheumatoid arthritis or multiple sclerosis, which composition comprises a chromophore in accordance with any of claims 1-21 that is adapted to be delivered to said diseased or undesired cells, and a suitable carrier.

39 Use of a chromophore in accordance with any of claims 1-21 in the production of a medicament, for use in the treatment of patients suffering from a disease or disorder which is characterised by the presence in the body of diseased or undesirable cells, such as tumours, cancers, viral infections including HIV infection, and autoimmune disorders including rheumatoid arthritis or multiple sclerosis; said chromophore being adapted for delivery to said diseased or undesired cells.

40 A method for separating a mixture which comprises one or more hydrophilic chromophores each having a hydrophilic or amphiphilic moiety, and a plurality of less hydrophilic substances and/or molecules, comprising the step of introducing said mixture to a hydrophobic eluting solvent, and passing said mixture and said eluting solvent over a hydrophilic or polar solid phase, such that said one or more chromophores are arrested on said solid phase whilst said substances and/or molecules are eluted or substantially eluted from said solid phase by said eluting solvent.

41 A method for the synthesis of a 5,10,15,20-tetra-meso-substituted porphyrin, chlorin or bacteriochlorin chromophore having selected substituents at the 5,10,15 and 20 meso-positions thereof; comprising the steps of providing a 5,15-di-meso-substituted porphyrin, chlorin or bacteriochlorin chromophore; attaching a leaving group Q to the 10 and 20 meso-positions of said chromophore, which leaving group Q is selected from halide and triflate; providing a coupling reagent (R₁₁O)(R₁₂O)BR₁₃, wherein R₁₁, and R₁₂ are independently selected from H or C₁₋₆ alkyl or R₁₁, and R₁₂ together constitute a C₁₋₆ alkyl chain bridging said two O atoms, and R₁₃ is vinyl or aryl, such as a hydrophilic aryl moiety as hereinbefore defined in relation to R₃; and reacting said chromophore with said coupling reagent in the presence of a base selected from potassium phosphate, sodium phosphate, caesium carbonate and barium hydroxide, and a Pd₀ catalyst; such that said R₁₃ replaces said leaving group Q at the 10- and 20-meso positions of said chromophore.

42 A method as claimed in claim 41, wherein said 5,15-di-meso-substituted porphyrin, chlorin or bacteriochlorin chromophore is a chromophore in accordance with any of claims 1-21, or a protected form thereof.

43 A method as claimed in claim 41 or claim 42, wherein said R₁₃ is vinyl, and said 5,10,15,20-tetra-meso-substituted porphyrin, chlorin or bacteriochlorin chromophore is subjected following said coupling reaction to an osmylation reaction utilising OsO₄, such as to convert said 10- and 20-vinyl substituents to hydroxyalkyl.
